(19) United States
(12) Patent Application Publication
(10) Pub. No.: US 2025/0205656 A1
(43) Pub. Date: Jun. 26, 2025

(54) APPARATUS FOR MIXING IONIZED HYDROGEN AND OXYGEN IONS WITH GAS-PHASE, LIQUID-PHASE, AND SOLID-PHASE SUBSTANCES AND TRANSFERRING QUANTUM ENERGY TO MIXTURES

(71) Applicant: Boo Yeol KIM, Siheung-si, Gyeonggi-do (KR)

(72) Inventor: Boo Yeol KIM, Siheung-si, Gyeonggi-do (KR)

(21) Appl. No.: 18/289,000

(22) PCT Filed: Apr. 25, 2022
(86) PCT No.: PCT/KR2022/005851

§ 371 Date: Sep. 30, 2024

(30) Foreign Application Priority Data
May 4, 2021 (KR) 10-2021-0057906

(51) Int. Cl. B01F 23/23; B01F 23/231; B01F 23/232; B01F 23/2326; B01F 23/237; B01F 23/80; B01F 25/312; B01F 25/51; B01F 33/81; B01F 35/71

(52) U.S. Cl. CPC B01F 23/238; B01F 23/231233; B01F 23/23231; B01F 23/2326; B01F 23/237612; B01F 23/23764; B01F 23/23765; B01F 23/805; B01F 25/31243; B01F 25/51; B01F 33/811; B01F 35/712; B01F 35/718051; B01F 2215/0431

(57) ABSTRACT

An apparatus, includes: a first raw material supply unit 110 including a filter housing 111, a supply fan 112, a flow regulator 113, an electronic valve 114, and an air supply line 115, wherein the supply fan 112 is operated to suck in external air, in the process, the HEPA filter (not shown) mounted inside the filter housing 112 filters fine dust and adjusts the air supply flow rate from the flow regulator 113 to the appropriate flow rate and supplies through the supply line 115 to the ion generator 200; a second raw material supply unit 120 including a pressure regulator 122, a flow regulator 123, an electronic valve 124, and an air supply line 125.

### TECHNICAL FIELD

The present invention includes hydrogen (H₂), oxygen (O₂), nitrogen (N₂), carbon dioxide (CO₂), and argon gas filled in a pressure vessel, and hydrogen gas is mixed with any one inert gas selected from nitrogen, carbon dioxide gas, or argon gas, and is diluted by 96% or more so that the hydrogen gas content in the air has less than 4% (the lower limit of the explosion). The diluted hydrogen gas is transferred to the ion generator or oxygen molecules are supplied to the ion generator to perform a high voltage discharge process in which quantum energy is irradiated.

Additionally, it is possible to generate hydrogen ions (H⁺, H⁻) or oxygen ions (O⁺, O⁻) by dissociating hydrogen molecules (H₂) or oxygen molecules (O₂) by irradiating the quantum energy generated by the braking radiation type quantum energy generator, and to supply the generated ions to the ion separator to separate hydrogen and oxygen ions (O⁺), or hydrogented ions (H⁻) and oxygen ions (O⁻).

Subsequently, each of the separated ions is supplied to the quantum energy transfer apparatus, mixed with the gaseous, liquid, or solid materials supplied to the quantum energy transfer apparatus, and then transferred quantum energy corresponding to a specific frequency to the mixture.

### BACKGROUND ART

Hydrogen is composed of the smallest atoms of the elements in nature, and the binary molecular state of two hydrogen atoms combined is a stable elemental state, and the molecular formula is H₂

Hydrogen is the first element in the periodic table with an atomic number of 1 and the element symbol H.

The main isotopes of hydrogen are deuterium (²H or D) and tritium (³H or T), in addition to prottium (¹H), which accounts for 99.98% of the hydrogen found on Earth.

Hydrogen is a colorless, odorless, and tasteless flammable gas at room temperature and 1 atm pressure, and when mixed with air at a volume ratio of 4 to 74%, it has the property of exploding due to external stimuli such as heat, flame, and sunlight. Since hydrogen can react with most non-metallic elements to form covalent bonds, most of the hydrogen on Earth is in the form of water or organic compounds. The positive ions of hydrogen are called protons (H⁺) and the negative ions are called hydride ions or hydride (H⁻).

American scientist Irving Langmuir (1881-1957) was awarded the Nobel Prize in Chemistry for his discovery of atomic hydrogen (H⁻). His work since the 1920s has clarified the existence of hydrogen and hydride ions in their atomic state.

Since water is normally dissociated into H2O↔H⁺+OH⁻, electrolysis collects H⁺ at the negative (−) electrode and hydrogen gas is generated there.

There is a negative hydrogen ion (H⁻) with two electrons in the outer orbital of the hydrogen atom.

The Japanese Iwanami (Dictionary of Science) also describes in detail the existence of negative hydrogen ions (H⁻).
1) Hydrogen atoms lose electrons when they come into contact with water, and they become protons (H⁺) when they are ionized.
2) When another electron is added to the hydrogen atom and it has two electrons, it becomes a hydride ion (H⁻)
3) Hydride ions (H⁻) have a strong reducing power compared to ordinary hydrogen atoms (H).

### Research on the Medical Uses of Hydrogen

1) Molecular hydrogen was announced in 2007 by Professor Odashigeo of Nippon Medical University in the world-renowned journal Nature Medicine that hydrogen acts as a therapeutic antioxidant that selectively reduces toxic free radicals.

In addition, in 2012, Prof. Odashi Geo once again announced that molecular hydrogen is a new antioxidant that can efficiently reduce oxidative stress (aging) and improve mitochondrial diseases in cells.

In addition, more than 500 academic papers have been published on the excellent efficacy of (molecular) hydrogen, and it has been announced that molecular hydrogen has excellent effects on human skin and the human body.

### 2) Supply of Hydride Ions (H⁻)

Normally, hydrogen has one proton in the nucleus and one electron spinning. However, there is only one proton, but one more electron can be added to form hydrogen with two electrons. The U.S. doctor, Patrick Planagan, named it a negative hydrogen ion. This negative hydrogen ion (H⁻) specially treated silicon and attached a negative hydrogen ion (H⁻), which is known to combine with water to emit a large amount of extra electrons when absorbed into the human body. The extra electrons in the human body act as antioxidants, so the treatment effect can be expected.

Recently, in Japan, an electronic supply unit is made and used as an antioxidant by attaching a negative hydrogen ion (H⁻) to coral calcium instead of silicon.

The quantum energy generator was designed by Tesla, and one coil is wound to the right and the other to the left on two cylinders without an iron core. Then, when power is supplied to each coil and the current flows in the opposite direction, a magnetic field is generated around the coil, and the two magnetic fields are overlapped and offset (dissipate), and the strength of the magnetic field is zero. It was discovered that strange energy appeared as a magnetic field was generated, and scientists referred to this energy as non-Hertzian wave, quantum energy, or scalar energy.

Mobius said that the ∞ method of winding two coils on a coreless cylinder was Mobius Coil. While the quantum energy field generated by the existing Tesla Coil has the same frequency as the introduced current, the improved Mobius Coil caused the generated quantum energy field to be generated as a set with a harmonic function complemented by amplification, offsetting, and interference at a specific frequency.

Since Tesla, a lot of research has been done on the coils of Tesla coils. The characteristic of the coil of the Tesla coil is that one is wound on the right and the other is wound on the left, and the direction of the current flows in the opposite direction, creating a magnetic field in the opposite direction, and the two magnetic fields cancel each other out, and the magnetic field is said to be zero. As the magnetic field is thus canceled, strange new energies emerge. Scientists called this energy the non-Hertzian wave, or scalar energy, or zero energy.

Aharonov (1959) proved mathematically that a magnetic field exists on the inside of a Tesla coil but disappears on the outside, leaving only quantum energy. In addition, Jennison said that Tesla Coil has a topological specificity that allows it to capture high-dimensional energy.

Jennison said that mathematical formulas show that two orthogonal standing observed. In addition, the interaction of the two standing waves depends on the winding shape of the coil and the distribution of the trapped field in the cavity. And the Tesla Coi could work like an antenna. In addition, Chambers (1960) said that quantum energy can be generated by sending direct current to the Tesla Coil, but quantum energy can also be generated by sending alternating current.

Georges Lakhovsky developed apparatus in France in the 1920s for cosmetic treatment, which he called the Multi-Wave Oscillator, which he built by attaching an antenna to a Tesla Coil.

According to Seiki (1990), a Moebius coil has half of the vectors facing up and half of the vectors facing down, so the sum the vectors is zero (0) when considered as a whole system. (Electromagnetic field is zero)

Seiki said that when the resistance loss of the Moebius coil was mathematically calculated, an imaginary electric field was found. The energy of this imaginary number corresponds to the imaginary part of the electrostatic scalar energy and the imaginary part of the magnetic field in Maxwell's electromagnetic equations. The electromagnetic field of an imaginary number is said to be a quantum energy field. Bearden also called an imaginary number electric fields scalar energy.

According to Johnson (1992), the Moebius coil is that the electromagnetic field causes the curvature of local space-time, that is, it destroys symmetry, so that higher-dimensional energy enters the three-dimensional world. In this way, the field of studying the topology of electromagnetism is called topological electronics.

Using the principles described above, various quantum energy generators have been created.

The types of quantum energy generators are as follows:
1. Tesla Coil
2. Mobius Coil or Caduceus Coil modified from Tesla Coil
3. Apparatus that combines Mobius Coildp frequency generator
4. Plasma Wave apparatus: Priore machine
5. Apparatus that combines Plasma Wave with a frequency generator

In other words, quantum energy, superquantum energy, scalar energy, non-Hertzian wave energy, etc., are used similarly, so we will just use them in the same sense in quantum medicine.

1. Korean Patent Publication No. 10-1011243 (nascent oxygen hydrogen water of electrolysis) relates to a water electrolysis hydrogen and oxygen generator for obtaining a mixed gas of hydrogen and oxygen by electrolyzing water. The water electrolysis device is installed and fixed in a water decomposition water tank to maintain a gap between a space gap part and a circular pole pin by connecting and fixing the space gap part and the circular pole pin with an insulator bar at four corners of an anode plate and a cathode plate while the circular pole pin is combined to face the inside by fixing a plurality of circular pole pins at regular intervals on the anode plate and the cathode plate connecting a power source. This technology generates less hydrogen than the present invention, consumes a lot of electricity because it is an electrolysis method, and does not have the function of generating quantum energy and irradiation.

2. In the Korean Published Patent No. 10-2021-0004721 (Quantum board and its temperature control device) it composed of a perlite panel, a quantum board consisting of a hito nano heating wire that is wired in Zigzag pattern inside the perlite panel and generates quantum waves with light energy corresponding to the high-density far-infrared spectrum, and a power supply unit that supplies power to the heating wire, and a setting unit that outputs a temperature setting signal by receiving a signal from the user to set the heating temperature and heating temperature for the heating wire of the quantum board, and a control unit that controls the heating operation according to the internal temperature and outside temperature of the perlite panel. This technology, which generates quantum waves with corresponding light energy and treats quantum waves with light energy corresponding to the far infrared spectrum in the quantum waves by transcribing them to the user's body, does not have the pulsating quantum energy transfer function generated in the state of zero magnetic field that is annihilated by overlapping pulsed electromagnetic field irradiation of various frequencies and pulsed electromagnetic fields of various frequencies in opposite directions, and does not have the quantum energy transfer function in the form of braking radiation, and has a limited scope of use.

3. In the Korean Patent Publication No. 10-2091512 (apparatus for emitting quantum energy for treatment and health management), a thermoelectron emission cathode with a filament is installed on the left side inside the sealed glass tube, a sealed glass tube (body) is installed on the left side of the cathode at a certain distance, and an anode made of a material such as rhodium is installed on the right side at a certain distance from the cathode, and a quantum energy emission layer made of material such as beryllium is formed on the external exposed surface of the anode, and the power is supplied to the cathode through a power supply unit, and the output of a variable high-voltage generator in the range of 10 to 30 kV is connected between the anode and the cathode. Thermal electrons are generated at the cathode by supplying power to the cathode, departing towards the anode receiving high-voltage power from a variable high-voltage generator, reaching the anode of rhodium, etc., and after impacting it, it collides with the extranucleus of the anode material, and a certain amount of kinetic energy is offset (dissipated) by the damping action, and the residual energy generates X-ray and quantum energy, and the X-ray and quantum energy penetrating the anode are converted into photoelectron and quantum energy as they are incidentally incident in the radial radiation layer of non-radial substances, and this technology adopts a monolithic cathode and a single anode, so the quantum energy generation is limited.

In other words, hydrogen and oxygen are generated and supplied using hydrogen and oxygen-generating raw materials, highly explosive hydrogen and inert gases such as nitrogen and carbon dioxide are mixed, high voltage discharge or quantum energy is irradiated in the ionization generator to ionize the mixed gas to generate negative hydrogen and oxygen ions, and then pulsed electromagnetic fields in various frequency bands are investigated, pulsed electromagnetic fields in opposite directions are overlapped and offset (dissipated), and pulsation quantum energy generated in the zero magnetic field, have not been disclosed in the technology developed so far.

### DISCLOSURE

### Technical Problem

The present invention was devised to solve the above-described problems.

A first raw material supply unit includes a filter housing, a supply fan, a flow regulator, an electronic valve, and an air supply line, and the supply fan is operated to suck in external air. In the process, the HEPA filter mounted inside the filter housing filters fine dust and adjusts the air supply flow rate from the flow regulator to the appropriate flow rate and supplies through the supply line to the ion generator; and

A second raw material supply unit includes a pressure regulator, a flow regulator, an electronic valve, and an air supply line, and oxygen gas is supplied to the pressure regulator by opening the main valve (not shown) of the container filled at high pressure (120 kg/cm²), depressurizing to the range of 50 mm Aq˜350 mm Aq in the pressure regulator, and supplying to the flow regulator, and after adjusting the flow rate at an appropriate flow rate in the flow regulator, supplied to the supply line through the electronic valve and supplied to the ion generator;

A third raw material supply unit includes a 3-1 raw material supply unit and a 3-2 raw material supply unit. The 3-1 raw material supply unit includes a main body, a stirrer, a motor, a first bevel gear, a second bevel gear, a rotating shaft, a first quantum energy generation coil, a second quantum energy generation coil, a power supply unit, storage tanks, an exhaust fan, a municipal water or purified water supply line and an exhaust line, and the storage tanks stores hydrogen generating raw materials such as borohydride salt (MBH₄) and triborohydride salt (MB₃H₈).

While stirring the municipal water or purified water stored in the main body by operating the stirrer, the first quantum energy generating coil installed on the outer circumferential surface of the rotating shaft of the stirrer and the second quantum energy generating coil installed inner circumferential surface of the main body.

Pulse-shaped power generated by the power supply unit is supplied to the first and the second quantum energy generation coil, so that the pulse-shaped electromagnetic field and the electromagnetic field in the opposite direction overlap and offset (dissipate) to produce pulsating quantum energy generated in a zero magnetic field state.

While the pulsating quantum energy is irradiating municipal water or purified water, the hydrogen generating raw materials stored in the storage tanks are supplied, and the hydrogen generated by the hydrolysis reaction with municipal water or purified water is supplied to a mixer using the exhaust fan. The 3-2 raw material supply unit includes a main body, a stirrer, a motor, a first bevel gear, a second bevel gear, a rotating shaft, a first quantum energy generation coil, a second quantum energy generation coil, a power supply unit, storage tanks, an exhaust fan, a municipal water or purified water supply line and an exhaust line.

The storage tank is stored oxygen generating raw materials such as sodium peroxide and potassium peroxide and the storage tank is stored catalyst materials such as ferric formate, ferric citrate, ferric ammonium citrate.

While stirring the municipal water or purified water stored in the main body by operating the stirrer, the first quantum energy generating coil installed on the outer circumferential surface of the rotating shaft of the stirrer and the second quantum energy generating coil installed on the inner circumferential surface of the main body. Pulse-shaped power generated by the power supply unit is supplied to the first and the second quantum energy generation coil, so that the pulse-shaped electromagnetic field and the electromagnetic field in the opposite direction overlap and offset (dissipate) to produce pulsating quantum energy generated in a zero magnetic field state.

While the pulsating quantum energy is irradiating municipal water or purified water, the oxygen generating raw materials stored in the storage tanks are and catalyst materials supplied, and the oxygen generated by the hydrolysis reaction with municipal water or purified water is supplied to an ion generator using the exhaust fan.

A fourth raw material supply unit includes a container filled with hydrogen gas, a pressure regulator, a flow regulator and an electronic valve. Hydrogen gas (H₂) is filled in container 141 with high pressure (120 kg/cm²), the main valve of the container is opened and supplied to the pressure regulator, and supplied to the flow regulator after depressurization in the range of 50 mm Aq˜350 mmAq, and after adjusting the flow rate to an appropriate flow rate in the flow regulator, it is supplied to the mixer through the supply line and mixed with the inert gas and then supplied to the ion generator.

A first inert gas supply unit includes a container filled with nitrogen gas, a pressure regulator, a flow regulator and an electronic valve. Nitrogen gas (N₂) is supplied to the pressure regulator after opening the main valve of the container filled with high pressure (120 kg/cm²), and after depressurizing the pressure regulator to the range of 50 mm Aq˜350 mmAq, supplying it to the flow regulator, and after adjusting the flow rate to the appropriate flow rate in the flow regulator, it is supplied to the mixer through the supply line.

A second inert gas supply unit includes a container filled with carbon dioxide gas, a pressure regulator, a flow regulator and an electronic valve. Carbon dioxide gas (CO₂) opens the main valve of the container filled with high pressure (120 kg/cm²) and feeds it to the pressure regulator, and after depressurizing the pressure regulator to the range of 50 mmAq˜350 mmAq, it is supplied to the flow regulator, and after adjusting the flow rate to the appropriate flow rate in the flow regulator, it is supplied to the mixer through the supply line.

A third inert gas supply unit includes a container filled with argon gas, a pressure regulator, a flow regulator and an electronic valve. Argon gas (Ar) opens the main valve of the container filled with high pressure (120 kg/cm²) and feeds it to the pressure regulator, and after depressurizing the pressure regulator to the range of 50 mmAq˜350 mmAq, it is supplied to the flow regulator, and after adjusting the flow rate to the appropriate flow rate in the flow regulator, it is supplied to the mixer through the supply line.

A first quantum energy generator includes a sealed glass tube, a thermal electron emission cathode, a first power supply unit supplying direct current power to the cathode, a first anode, a second power supply unit to apply high voltage to the first anode, a target plate installed in contact with the first anode, a second anode, a third power supply unit to supply direct current high-voltage power to the second anode and a quantum energy divergent layer.

The direct current power generated by the first power supply unit is supplied and heated to the thermal electron emission cathode, and then thermal electrons are emitted from the thermal electron emission cathode.

The high voltage generated by the second power supply unit is supplied to the first anode, and then the thermal electrons emitted from the negative electrode by a bias circuit configured between the first power supply unit and the second power supply unit accelerates in the direction of the first anode and collides with the target plate and refracts at a 90 degree angle downward.

The high voltage generated by the third power supply unit is supplied to the second anode, and then the thermal electrons collided with the target plate accelerates in the direction of the second anode by a bias circuit configured between the second power supply unit and the third power supply unit, after being impacted by the collision with the extranucleus electrons of the second anode material, a certain amount of kinetic energy is dissipated by the damping action, and the residual energy generates X-ray and quantum energy with waves. The X-ray and quantum energy penetrating the anode are converted into photoelectron and quantum energy with waves as they are irradiated to the emission layer of non-radial matter, and the quantum energy generated by the radiation moderation phenomenon is irradiated inside of a main chamber.

The gases such as hydrogen, oxygen, carbon dioxide are supplied from the raw material supply unit and passes through the inside of the main chamber, the quantum energy generated by the radiation moderation phenomenon is irradiated to the gases. By quantum energy irradiation, the covalent bonds of the gases material are dissociated, and the gases material may become atoms, cations or anions.

A first ion generator includes the first quantum energy generator of the braking radiation type.

A second quantum energy generator includes a sealed glass tube, a cathode, a first power supply unit supplying direct current power to the cathode, an electron emission source, a gate electrode, a first anode, a second power supply unit to apply high voltage to the first anode, a X-ray target plate installed in the center of the right slope of the first anode, a second anode, a third power supply unit to supply direct current high-voltage power to the second anode and a quantum energy divergent layer installed in contact with the surface of the second anode.

When the direct current power generated by the first power supply unit is supplied to the cathode, the electron beams generated by the electron emission source installed in contact with the cathode are accelerated in the direction of the first anode by the bias circuit configured between the first power supply unit and the second power supply unit,
and the electron beams emitted and refracted X-rays by collision with the target surface of the tungsten (W) material attached to the right slope of the first anode is accelerated toward the second anode by the bias circuit configured between the second power supply unit and the third power supply unit. And then the electron beams is reached the second anode of rhodium, etc., and collided with the nuclear extraelectronic (extranuclear) of the anode material, a certain amount of kinetic energy is dissipated by the decay action, and the residual energy generates X-ray and quantum energy with waves. The X-ray and quantum energy penetrating the anode are converted into photoelectron and quantum energy with waves as they are irradiated to the emission layer of non-radial matter, and the quantum energy generated by the radiation moderation phenomenon is irradiated inside of a main chamber.

The gases such as hydrogen, oxygen, carbon dioxide are supplied from the raw material supply unit and passes through the inside of the main chamber, the quantum energy generated by the radiation moderation phenomenon is irradiated to the gases. By quantum energy irradiation, the covalent bonds of the gases material are dissociated, and the gases material may become atoms, cations or anions.

A second ion generator includes the second quantum energy generator of the braking radiation type,
A third quantum energy generator of high-voltage discharge method includes a main body (a housing), a first saddle gradient coil having first discharge electrodes, a second gradient saddle coil having second discharge electrodes, a modified Trigger coil having a ground electrode, a modified Trigger coil having third discharge electrodes, a high-voltage generator and a conducting wire.

After insulating the inner surface of the main body with an insulator, the first gradient coil having the first discharge electrodes and the second gradient saddle coil having the second discharge electrode is installed in surface contact with the inner surface of the main body, and are installed to face each other at upper and lower intervals, and the wound coil directions are opposite to each other.

The modified Trigger coil having the ground electrode is installed at intervals from Gradient saddle coil in the inner center direction.

The modified Trigger coil having the third discharge electrode is installed at intervals from the modified Trigger coil in the inner center direction.

In the high-voltage generator installed on the outside side, when the generated pulsed high-voltage is supplied to the first gradient saddle coil having the first discharge electrodes, the second gradient saddle coil having the second discharge electrodes, the modified Trigger coil having the ground electrodes, and the modified Trigger coil having the third discharge electrodes, between the coils, the electromagnetic field of the pulse form of the opposite direction is generated, and the electromagnetic field of the pulse form of the opposite direction at the center distance is superimposed and extinguished, and pulsating orient energy is generated in the state of the zero magnetic field, and at the same time it is irradiated inside the main body, and at the same time a high-voltage discharge is initiated between the coils.

The gases such as hydrogen, oxygen, carbon dioxide are supplied from the raw material supply unit and passes through the inside of the main chamber, the quantum energy and the high-voltage discharge energy are irradiated to the gases.

By quantum energy irradiation and the high-voltage discharge energy, the covalent bonds of the gases material are dissociated, and the gases material may become atoms, cations or anions.

A third ion generator includes the third quantum energy generator of the high-voltage discharge method.

An ion generator includes the first ion generator, the second ion generator and the third ion generator.

A first dissolving device includes a first circulating pump, a flow control valve, a venturi ejector, a pressure detection sensor, a chamber, a first defoamer and a circulation tube.

The first defoamer includes a first quantum energy generator consisting of a first quantum energy generating coil, a second quantum energy generating coil and a pulsed power supply unit. By operating the first circulation pump, the solution stored in a main body of a quantum energy transfer device is sucked and pressurized, and hydrogen ions (H⁺, H⁻), oxygen ions (O⁺, O⁻) or nitrogen ions (N⁺, N⁻, NO) generated in the ion generator are supplied in the process of passing through the venturi ejector.

Air bubbles mixed in the solution are primarily destroyed and decompressed during the process of passing through a 1 mm pore disperser installed in the inner center of the main body by supplying hydrogen ions (H⁺, H⁻), oxygen ions (O⁺, O⁻) or nitrogen ions (N⁺, N⁻, NO) into the main body.

In the pulse-type power supply unit installed on the outside side, the generated pulsed high-voltage is supplied to the first and second quantum energy generating coils of Uniform saddle coil.

The first and second quantum energy generating coils are installed to face each other at interval, and the wound coil directions are opposite to each other.

When supplied to the first and the second quantum energy generating coils, the magnetic field and electromotive force in the form of pulses in opposite directions generated at an angle of 90 degrees in the direction of current flow act on the solution flowing inside the body, and the magnetic field in the form of pulses in opposite directions overlap and offset (dissipate) in the center of the body, and quantum energy is generated in the state of zero magnetic field, and irradiated to the solution inside the body to depocalyse (destroy) the air bubbles contained in the solution, and the hydrogen ions (H⁺, H⁻), oxygen ions (O⁺, O⁻) or nitrogen ions (N⁺, N⁻, NO) are primary dissolved.

A second dissolving device includes a second circulating pump, a flow control valve, a pressure detection sensor, a chamber, a second defoamer and a circulation tube. The second defoamer includes a second quantum energy generator consisting of a first quantum energy generating coil, a second quantum energy generating coil and a pulsed power supply unit. When a control panel supplies power to the second circulation pump, the second circulation pump is operated so that the process fluid in which hydrogen ions (H⁺, H⁻), oxygen ions (O⁺, O⁻) or nitrogen ions (N⁺, N⁻, NO) are primarily dissolved in the first dissolving device is sucked and pressurized by the second circulation pump and introduced into the second defoamer to which the second quantum energy is irradiated through the flow rate adjusting valve.

The chamber of the second dissolving device to which the second quantum energy is irradiated has a cylindrical shape having a predetermined diameter and a predetermined length, and is insulated from the inner surface of the cylindrical chamber so that a first quantum energy generation coil having Solenoid coil shape is installed to be in surface contact with the inner surface. A second quantum energy generation coil having Trigger coil shape is installed at intervals, and a third quantum energy generation coil having a modified RF coil shape is installed downward at intervals from the first and second quantum energy generation coils.

Trigger voltage generator is installed at one side of the outside of the main body, is connected to the pulsed power supply unit with a conducting wire.

The first and second quantum energy generating coils are connected to a pulsed power supply unit through a conducting wire, and the third quantum energy generating coil is connected to the Trigger voltage generator through the conducting wire. The pulsed power supply unit and the Trigger voltage generator are also connected through a conducting wire.

The third quantum energy generation coil starts discharging while pulse energy is transferred between the first and second quantum energy generation coils having polarities different from those of the magnetic field. The third quantum energy generation coil starts main discharge at a lower voltage than the high voltage pulse applied to the first quantum energy generation coil and the second quantum energy generation coil. At the same time, when a high voltage pulsed power supply unit generates a pulse-type high voltage to the first quantum energy generation coil and the second quantum energy generation coil installed such that winding directions of the coils are opposite to each other through a conducting wire, a pulse-type magnetic field in opposite directions is generated in the first quantum energy generation coil and the second quantum energy generation coil at an angle of 90° with respect to a flow direction of a current, pulse-type magnetic fields in opposite directions are overlapped and offset (dissipated) at a center distance of the first quantum energy generation coil and the second quantum energy generation coil, and quantum energy is generated in a zero magnetic field state and irradiated to the introduced fluid.

As a result, a shock wave is generated by the discharge, and at the same time, bubbles containing hydrogen ions (H⁺, H⁻), oxygen ions (O⁺, O⁻) or nitrogen ions (N⁺, N⁻, NO), which are not destroyed in the first dissolving device passing through between the first quantum energy generation coil, the second quantum energy generation coil, and the third quantum energy generation coil, are destroyed by the discharge shock wave, and the water hammer pressure generated by the destruction of the bubbles efficiently further destroys the bubbles adjacent to the discharge shock wave.

A first ion separator includes a positive charge collector and a first quantum energy generation device. The positive charge collector includes a main body, a first power supply unit, a coil and multiple positive charge collecting electrodes. The main body of the first ion separator may have a cylindrical shape with conical upper and lower parts, or a rectangular parallelepiped shape with rectangular pyramid shapes at upper and lower parts.

A plurality of first positive charge collecting electrodes supplied with power generated by a power supply unit of the positive charge collector are installed in upper and lower holders at intervals from each other inside the main body and at intervals from the upper, lower, left, and right inner surfaces of the main body.

The positive charge collector includes the power supply unit, the first coil, the positive charge collecting electrodes, and a conducting wire.

In addition, a first Cuffs coil of the first quantum energy generator is installed in outer upper area of the main body, a second Cuffs coil is installed at in outer lower area of the main body, and a power supply unit is installed at an interval to supply the power generated from the power supply unit to the first Cuffs coil and the second Cuffs coil through a conducting wire.

Pulse-type variable power is supplied to the first Cuffs coil and the second Cuffs coil wound in opposite directions through a wire from the power supply unit of the first quantum energy generator. In the first Cuffs coil, a pulsed electromagnetic field (PEMF) is irradiated in the lower direction of the main body at an angle of 90 degrees with the current flow direction, and in the second Cuffs coil, a pulsed electromagnetic field (PEMF) is irradiated in the upper direction of the main body at an angle of 90 degrees with the current flow direction. As a result, the pulse-type magnetic fields irradiated in opposite directions at the central distance between the first Cuffs coil and the second Cuffs coil overlap and offset (dissipate), and the pulsation quantum energy generated in the zero magnetic field state is irradiated into the central distance space between the first Cuffs coil and the second Cuffs coil inside the main body.

In the positive charge collector, the output positive (+) terminal of the power supply unit is directly connected to the first coil through a conducting wire, but the output negative (−) terminal of the power supply unit is connected to the plurality of first positive charge collecting electrodes through a conducting wire, and each of second positive charge collecting electrodes is separated from the first positive charge collecting electrodes at an interval so that current does not flow.

The first coil is directly supplied with positive (+) power of the power supply unit, and negative (−) power is not supplied to the first coil installed to be connected to the second positive charge collecting electrodes installed separately from the first positive charge collecting electrodes connected to the output negative (−) terminal of the power supply unit. Therefore, between the first positive charge collecting electrodes and the second positive charge collecting electrodes, there is a strong attraction to supply negative (−) power.

As a result, hydrogen ions (H⁺, H⁻), oxygen ions (O⁺, O⁻) or nitrogen ions (N⁺, N⁻, NO) generated by dissociating hydrogen molecules, oxygen molecules or nitrogen molecules in the ion generators are introduced into the first ion separator and pass through between the first and second collection electrodes, and hydrogen ions (H⁺), oxygen ions (O⁺) or nitrogen ions (N⁺) are adsorbed (captured) to the first charge collecting electrodes through which negative (−) currents flow and the second charge collecting electrodes in which attraction acts.

On the contrary, hydride ions (H⁻), oxygen ions (O⁻) or nitrogen ions (N⁻) may be supplied to the energy transfer device by the pressing force of the second circulation pump by acting a repulsive force with the first charge collecting electrodes through which negative (−) current flows.

The second ion separator includes a negative charge collector and a second quantum energy generaton device. The negative charge collector includes a main body, a first power supply unit, a coil and multiple negative charge collecting electrodes. And the second quantum energy generaton device includes a first Cuffs coil, a second Cuffs coil and a power supply unit.

The second ion separator includes a negative charge collector and a second quantum energy generaton device. The negative charge collector includes a main body, a first power supply unit, a coil and multiple negative charge collecting electrodes. And the second quantum energy generaton device includes a first Cuffs coil, a second Cuffs coil and a power supply unit.

The main body of the second ion separator may have a cylindrical shape with conical upper and lower parts, or a rectangular parallelepiped shape with rectangular pyramid shapes at upper and lower parts.

The plurality of first negative charge collecting electrodes supplied with power generated by the power supply unit of the negative charge collector are installed in the upper and lower holders at intervals from each other inside the main body and at intervals from the upper, lower, left, and right inner surfaces of the main body.

In addition, the first Cuffs coil of the second quantum energy generator is installed in the outer upper area of the main body, the second Cuffs coil is installed in the outer lower area of the main body, and the power supply unit is installed at an interval to supply the power generated from the power supply unit to the first Cuffs coil and the second Cuffs coil through the conducting wire.

Pulse-type variable power is supplied to the first Cuffs coil and the second Cuffs coil wound in opposite directions through a wire from the power supply unit of the second quantum energy generator. In the first Cuffs coil, a pulsed electromagnetic field (PEMF) is irradiated in the lower direction of the main body at an angle of 90 degrees with the current flow direction, and in the second Cuffs coil, a pulsed electromagnetic field (PEMF) is irradiated in the upper direction of the main body at an angle of 90 degrees with the current flow direction.

As a result, the pulse-type magnetic fields irradiated in opposite directions at the central distance between the first Cuffs coil and the second Cuffs coil overlap and offset (dissipate), and the pulsation quantum energy generated in the zero magnetic field state is irradiated into the central distance space between the first Cuffs coil and the second Cuffs coil inside the main body.

In the negative charge collector, the output negative (−) terminal of the power supply unit is directly connected to the second coil through a conducting wire, but the output positive (+) terminal of the power supply unit is connected to a plurality of first charge collecting electrodes through a conducting wire, and each of second charge collecting electrodes is separated from the first charge collecting electrodes at an interval so that current does not flow.

The second coil is directly supplied with negative (−) power of the power supply unit, and positive (+) power is not supplied to the second coil installed to be connected to the second charge collecting electrodes installed separately from the first charge collecting electrodes connected to the output positive (+) terminal of the power supply unit. Therefore, between the first charge collecting electrodes and the second charge collecting electrodes, there is a strong attraction to supply positive (+) power.

As a result, hydrogen ions (H⁺, H⁻), oxygen ions (O⁺, O⁻) or nitrogen ions (N⁺, N⁻, NO) generated by dissociating hydrogen molecules, oxygen molecules or nitrogen molecules in the first, second, and third ion generators are introduced into the first and second ion separators and pass through between the first and second collection electrodes, and hydrogen ions (H⁻), oxygen ions (O⁻) or nitrogen ions (N⁻) are adsorbed (captured) to the first charge collecting electrodes through which positive (+)) currents flow and the second charge collecting electrodes in which attraction acts.

A third ion separator includes a reactor, a condenser and a positive charge collector. The reactor includes a main body, a jacket, a cooling water circulation line, a hot water circulation line, a municipal water or purified water supply line, a raw material input line, and a stirrer. The lower part of the main body of the reactor is conical, the upper part of the main body is inclined, and the main body may have a dual-structure cylindrical shape including a jacket. The cooling water supply and exhaust line, and the hot water supply and exhaust line may be connected and installed on one side of the side lower portion and one side of the side upper portion of the jacket.

The condenser includes a main body, a cooling water circulation line, and a exhaust line of hydrogen ions (H⁻) or oxygen ions (O⁻).

The positive charge collector includes a plurality of first cathodes, a first coil (the load), a second power supply unit, and conducting wires.

The electronic valve of the municipal water or the purified water supply line is opened and the municipal water or the purified water is filled with 60 to 75% of the volume of the main body of the container. The electronic valve of the hot water supply and exhaust line is opened, and the hot water are circulated from the lower supply line of the jacket to the top exhaust line and heat the municipal water or the purified water filled with appropriate amount inside the main body to 40 to 60 degrees Celsius. The mixer is operated, and the mixer stirs the municipal water or the purified water in the range of 30 to 60 RPM. The pulse-type DC power generated by the power supply unit is supplied to the first electrode and the second electrode installed on the inner side surface of the reactor. The cover of the hopper is opened, and the metal hydrate in which one kind material is selected from the lithium hydride (LiH), the sodium hydride (NaH) and the potassium hydride (KH) is supplied to the gravity difference to the main body. Hydride ions (H⁻) may be generated by a hydrolysis reaction and an electrolysis reaction between the metal hydrate and the water. As the pulse-type DC power generated by the first power supply unit is supplied to the first electrode and the second electrode, the metal hydrate introduced into the main body is subjected to an electrolysis reaction to generate a hydrogen ions (H⁻).

Hydrogen ions (H⁻) generated by the hydration reaction flow into the main body of the condenser of a shell and tube shape through the supply line and are cooled by indirect heat exchange with cooling water introduced into the cooling water supply line and the exhaust line installed on one side of the lower and upper sides of the body to condense and liquefy the water vapor of water molecules mixed with hydrogen ions (H⁻). The liquefied water is discharged to the outside through a condensate exhaust line installed under the left cell of the main body so that hydrogen ions (H⁻) and water vapor are separated and introduced into the quantum energy transfer devices. In this process, positive charges may be collected on the first and second cathodes to suppress a reaction between hydride ions (H⁻) and a small amount of lithium ions (Li⁺) or sodium ions (Na⁺) and potassium ions (K⁺).

Specifically, negative (−) terminals on the output side of the power supply unit of the positive charge collector are connected to each of the plurality of first negative electrodes of the positive charge collector, positive (+) terminals are connected to the first coil, and another electric wire of the first coil is connected to the second negative electrodes. The second negative electrodes are installed to be separated from the first negative electrodes.

Negative (−) power is supplied to the first negative electrodes, and no power is supplied to the second negative electrodes, therefore, between the first negative electrodes and the second negative electrodes, there is a strong attraction to supply negative (−) power.

Therefore, lithium ions (Li⁺), sodium ions (Na⁺), and potassium ions (K⁺) are collected in the first and second cathodes inside the condenser, and hydrogen ions (H⁻) are separated from the cations by repulsive force and introduced into the quantum energy transfer devices by the pressing force of the air FAN.

A first quantum energy transfer device includes an ion gas supply line, a municipal water or purified water supply line, a solution supply line in which ions are dissolved, a circulation and exhaust lines, a first main body, a second main body, a stirrer, a circulation pump, a first quantum energy generation device, and a second quantum energy generation device. The first quantum energy generation device includes a first quantum energy generation coil, a second quantum energy generation coil, a first power supply unit for supplying power in a pulse form, and a conducting wire. The second quantum energy generation device includes a third quantum energy generation coil, a fourth quantum energy generation coil, a second power supply unit for supplying power in a pulse form, and a conducting wire.

A solution containing hydrogen ions (H⁺, H⁻) or oxygen ions (O⁺, O⁻) in the dissolving device is sent to the supply line by the pressing force of the pressure pump, followed by 70-80% of the internal volume of the main body through the venturi ejector.

Alternatively, a first solution in which hydrogen ions (H⁻) or oxygen ions (O⁻) supplied from the first and third ion separators or hydrogen ions (H⁺) or oxygen ions (O⁺) supplied from the secondary ion separator are mixed with purified water (municipal water) may be filled to 70 to 80% of the internal volume of the main body 501 through the supply line and the venturi ejector.

Alternatively, a second solution in which hydrogen ions (H⁻) or oxygen ions (O⁻) supplied from the first and third ion separators or hydrogen ions (H⁻) or oxygen ions (O⁻) supplied from the secondary ion separator 400B are mixed with purified water (municipal water) may be filled to 70 to 80% of the internal volume of the main body through the supply line and the venturi ejector.

In this process, the level sensor detects the water level and transmits it to the control panel, and the control panel stops the pressure pump and supplies power to the stirrer to rotate the first solution or the second solution supplied to the main body.

At the same time, when pulsed direct current power is supplied from the first power supply to the first and second quantum energy generating coils wound in opposite directions through a wire, an electromagnetic field in the form of a pulse is generated at an angle of 90 degrees in the current flow direction and transferred to the first solution or the first solution.

As a result, electromagnetic fields in the form of pulses in opposite directions overlap and offset (dissipate) in the center of the first body, generating quantum energy in a zero magnetic field state, and the quantum energy above can be irradiated to the solution.

Subsequently, when power is supplied to the circulation pump from the control panel, the solution irradiated with electromagnetic fields and quantum energy is sucked and pressurized inside the first body and supplied to the second body.

The third quantum energy generating coils and the fourth quantum energy generating coils may be wound in opposite directions, and an inlet and an outlet hole may be perforated between the coil and the coil wound with a predetermined winding number.

The pulse-type power generated by the second power supply unit is applied to the third quantum energy generating coils, in which the coils are wound in opposite directions, and the pulse-type electromagnetic field generated at an angle of 90 degrees in the current flow direction is installed to face each other. In the addition, opposite pulse-type electromagnetic energy generated between the third quantum energy generating coils and the fourth energy generating coils may be irradiated to the solution passing through the inlet and outlet holes. The electromagnetic energy in the form of pulses in opposite directions overlap and offset (dissipate), and the pulse-type quantum energy generated in the zero magnetic field state is irradiated and continues to circulate for a certain period of time, before the electric valve installed on the circulation line above the second body is closed and the electric valve is opened to supply the solution to which the electromagnetic field and quantum energy are irradiated.

A second quantum energy transfer device includes a main body, an ion gas supply line, a first quantum energy generation device, an upper first rotating body motor, a pneumatic cylinder, a first rotating disk, a lower second rotating body motor, a second rotating disk, and a second quantum energy generation device. The first quantum energy generation device includes a first quantum energy generation coil, a second quantum energy generation coil, a first power supply unit, and a conducting wire. The second quantum energy generation device includes a third quantum energy generation coil, a fourth quantum energy generation coil, a pulse-type power supply unit, and a conducting wire.

When the control panel supplies power to the motor for the second rotating body, the motor for the first rotating body, and the pneumatic or hydraulic cylinder, the motor of the second rotating body is operated to rotate the motor shaft and the second rotating disk.

At the same time, the pneumatic or hydraulic cylinder installed at the center of the upper portion of the main body is operated to lower the first rotating body composed of the motor, the shaft, and the turntable to the close distance (10 to 20 cm) of the quantum energy treatment object loaded on the turntable of the second rotating body inside the main body,
and then the motor of the first rotating body is operated in the control panel to rotate the shaft connected to the motor shaft and the turntable to rotate the hydrogen ion (H⁻) or oxygen ions (O⁻) supplied from the first ion separator, or oxygen ions (O⁺), the hydrogen ion (H⁺), the nitrogen ions (N⁺) or the nitrogen oxide ions (NO) supplied from the second ion separator in a state where the ions are supplied or not supplied to one side of the upper portion of the main body of the second quantum energy transfer device by the pressing force of the air FAN.

At the same time, the pulse-shaped electromagnetic fields generated by the first power supply unit are irradiated from the upper portion to the lower portion and from the lower portion to the upper portion to the loaded quantum energy transfer target in the first quantum energy generation coil of the plurality of first quantum energy generators installed to be in surface contact with the rear surface of the turntable of the first rotating body and the second quantum energy generation coil installed to be in surface contact with the rear surface of the turntable of the second rotating body so that the pulse-shaped electromagnetic fields generated by the first power supply unit are overlapped and offset (dissipated) in the center direction of the loaded target and transferred to the loaded object.

A third quantum energy transfer device includes a main body, a screw driving motor, a screw, a powder inlet, a powder outlet, a supply fan, an ion gas supply line and a third quantum energy generation device. The third quantum energy generation device includes a plurality of first quantum energy generation coils, a plurality of second quantum energy generation coils, a first power supply unit, and a conducting wire.

Hydride ions (H⁻), oxygen ions (O⁻), nitrogen ions (N⁻), nitrogen oxide ions (NO), hydrogen ions (H⁺), oxygen ions (O⁺) or nitrogen ions (N⁺) supplied from the ion separators, may be supplied to the venturi neck part (powder outlet) connected to the supply FAN of the third quantum energy transfer device by the suction power of the supply FAN or by the pressing force of the air FAN.

At the same time, when the quantum processing object (powder) is introduced into the main body by the blade of the supply FAN, and discharged to the outlet by the screw driven by the drive motor, the pulse-type power generated by the pulse-type power supply unit is supplied through the wire to the multiple first quantum energy generating coils and second quantum energy generating coils installed to face each other in the circumferential direction of the outer circumferential surface of the main body, an electromagnetic field in the form of a pulse generated at a 90 degree angle in the current flow direction is irradiated from the top to the bottom of the main body in the first quantum energy generating coil, and the second quantum energy generating coil is irradiated from the bottom of the main body to the top of the main body, and the pulse-type electromagnetic fields in opposite directions overlap and offset (dissipate) in the screw shaft portion to generate quantum energy in a zero magnetic field state.

When oxygen ions (O⁺, O⁻) are supplied from the ion gas supply line while the generated quantum energy is irradiated to the quantum energy treatment object, the powder raw material may be mixed with oxygen ions (O⁺, O⁻) by screw driving inside the main body to be perform an oxidation reaction.

When hydride ions (H⁻) or hydrogen ions (H⁺) are supplied from the ion gas supply line, the powder raw material may be mixed with hydride ions (H⁻) or hydrogen ions (H⁺) by screw driving inside the main body to be perform a reduction reaction.

When nitrogen ions (N⁺, N⁻, NO) is supplied from the ion gas supply line, the powder raw material may be mixed with the nitrogen ions (N⁺, N⁻, NO) by screw driving inside the main body to be perform a nitrification reaction.

### Technical Solution

In accordance with the present invention, the above and other objects can be accomplished by the provision of,
A first raw material supply unit 110 includes a filter housing 111, a supply fan 112, a flow regulator 113, an electronic valve 114, and an air supply line 115, and the supply fan 112 is operated to suck in external air. In the process, the HEPA filter (not shown) mounted inside the filter housing 112 filters fine dust and adjusts the air supply flow rate from the flow regulator 113 to the appropriate flow rate and supplies through the supply line 115 to the ion generator 200;
A second raw material supply unit 120 includes a pressure regulator 122, a flow regulator 123, an electronic valve 124, and an air supply line 125, and oxygen gas (O₂) is supplied to the pressure regulator 122 by opening the main valve (not shown) of the container filled at high pressure (120 kg/cm²), depressurizing to the range of 50 mm Aq˜350 mm Aq in the pressure regulator 122, and supplying to the flow regulator 123, and after adjusting the flow rate at an appropriate flow rate in the flow regulator 123, supplied to the supply line 125 through the electronic valve 124 and supplied to the ion generator 200;
A third raw material supply unit 130 includes a 3-1 raw material supply unit 130a and a 3-2 raw material supply unit 130b. The 3-1 raw material supply unit (130a) includes a main body 131, a stirrer 132, a motor 132a, a first bevel gear 132b, a second bevel gear 132c, a rotating shaft 132d, a first quantum energy generation coil 133, a second quantum energy generation coil 134, a power supply unit 135, storage tanks 136a and 136b, an exhaust fan 137, a municipal water or purified water supply line 138 and an exhaust line 139, and the storage tanks 136a and 136b store hydrogen generating raw materials such as borohydride salt (MBH₄) and triborohydride salt (MB₃H₈).

The first quantum energy generating coil 133 is installed on the outer circumferential surface of the rotating shaft 132d of the stirrer, and the second quantum energy generating coil 134 is installed on the inner circumferential surface of the main body 131.

While stirring the municipal water or purified water stored in the main body 131 by operating the stirrer 132, pulse-shaped power generated by the power supply unit 135 is supplied to the first and the second quantum energy generation coil 134. As a result, the pulse-shaped electromagnetic field and the electromagnetic field in the opposite direction overlap and offset (dissipate) to produce pulsating quantum energy generated in a zero magnetic field state.

While the pulsating quantum energy is irradiating municipal water or purified water, the hydrogen generating raw materials stored in the storage tanks 136a, 136b are supplied, and the hydrogen generated by the hydrolysis reaction with municipal water or purified water is supplied to a mixer 154 using the exhaust fan 137.

The 3-2 raw material supply unit 130b includes a main body 131, a stirrer 132, a motor 132a, a first bevel gear 132b, a second bevel gear 132c, a rotating shaft 132d, a first quantum energy generation coil 133, a second quantum energy generation coil 134, a power supply unit 135, storage tanks 136c and 136d, an exhaust fan 137, a municipal water or purified water supply line 138 and an exhaust line 139.

The storage tank 136c is stored oxygen generating raw materials such as sodium peroxide and potassium peroxide, and the storage tank 136d is stored catalyst materials such as ferric formate, ferric citrate and ferric ammonium citrate.

The first quantum energy generating coil 133 is installed on the outer circumferential surface of the rotating shaft 132d of the stirrer, and the second quantum energy generating coil 134 is installed on the inner circumferential surface of the main body 131.

While stirring the municipal water or purified water stored in the main body 131 by operating the stirrer 132, pulse-shaped power generated by the power supply unit 135 is supplied to the first and the second quantum energy generation coil 134. As a result, the pulse-shaped electromagnetic field and the electromagnetic field in the opposite direction overlap and offset (dissipate) to produce pulsating quantum energy generated in a zero magnetic field state.

While the pulsating quantum energy is irradiating municipal water or purified water, the oxygen generating raw materials and catalyst materials stored in the storage tanks 136c, 136d are supplied, and the oxygen generated by the hydrolysis reaction with municipal water or purified water is supplied to an ion generator 200 using the exhaust fan 137.

A fourth h raw material supply unit 140 includes a container 141 filled with hydrogen gas, a pressure regulator 142, a flow regulator 143 and an electronic valve 144. Hydrogen gas (H₂) is filled in container 141 with high pressure (120 kg/cm²), the main valve (not shown) of the container 141 is opened and supplied to the pressure regulator 142, and supplied to the flow regulator 143 after depressurization in the range of 50 mm Aq˜350 mmAq, and after adjusting the flow rate to an appropriate flow rate in the flow regulator 143, it is supplied to the mixer 154 through the supply line and mixed with the inert gas and then supplied to the ion generator 200.

A first inert gas supply unit 151 includes a container 151a filled with nitrogen gas, a pressure regulator 151b, a flow regulator 151c and an electronic valve 151d. Nitrogen gas (N₂) is supplied to the pressure regulator 151b after opening the main valve of the container 151a filled with high pressure (120 kg/cm²), and after depressurizing the pressure regulator 151b to the range of 50 mm Aq˜350 mmAq, supplying it to the flow regulator 151 c, and after adjusting the flow rate to the appropriate flow rate in the flow regulator 151c, it is supplied to the mixer 154 through the supply line.

A second inert gas supply unit 152 includes a container 152a filled with carbon dioxide gas, a pressure regulator 152b, a flow regulator 152c and an electronic valve 152d. Carbon dioxide gas (CO₂) is supplied to the pressure regulator 152b after opening the main valve of the container 152a filled with high pressure (120 kg/cm2), and after depressurizing the pressure regulator 152b to the range of 50 mm Aq˜350 mmAq, supplying it to the flow regulator 152c, and after adjusting the flow rate to the appropriate flow rate in the flow regulator 152c, it is supplied to the mixer 154 through the supply line.

A third inert gas supply unit 153 includes a container 153a filled with argon gas, a pressure regulator 153b, a flow regulator 153c and an electronic valve 153d. Argon gas (Ar) is supplied to the pressure regulator 153b after opening the main valve of the container 153a filled with high pressure (120 kg/cm2), and after depressurizing the pressure regulator 153b to the range of 50 mm Aq˜350 mmAq, supplying it to the flow regulator 153c, and after adjusting the flow rate to the appropriate flow rate in the flow regulator 153c, it is supplied to the mixer 154 through the supply line.

A first quantum energy generator 210 includes a sealed glass tube 201, a thermal electron emission cathode 204, a first power supply unit 202 supplying direct current power to the cathode, a first anode 213, a second power supply unit 211 to apply high voltage to the first anode 213, a target plate 213a installed in contact with the first anode 213, a second anode 223, a third power supply unit 221 to supply direct current high-voltage power to the second anode 223 and a quantum energy divergent layer 224.

The direct current power generated by the first power supply unit 202 is supplied and heated to the thermal electron emission cathode 204, and then thermal electrons are emitted from the thermal electron emission cathode 204.

The high voltage generated by the second power supply unit 211 is supplied to the first anode 213, and then the thermal electrons emitted from the negative electrode 204 by a bias circuit configured between the first power supply unit 202 and the second power supply unit 211 accelerates in the direction of the first anode 213 and collides with the target plate 213a and refracts at a 90 degree angle downward.

The high voltage generated by the third power supply unit 221 is supplied to the second anode 223, and then the thermal electrons collided with the target plate 213a accelerates in the direction of the second anode 223 by a bias circuit configured between the second power supply unit 211 and the third power supply unit 221, after being impacted by the collision with the extranucleus electrons of the second anode 223 material, a certain amount of kinetic energy is dissipated by the damping action, and the residual energy generates X-ray and quantum energy with waves.

The X-ray and quantum energy penetrating the anode are converted into photoelectron and quantum energy with waves as they are irradiated to the emission layer 224 of non-radial matter, and the quantum energy generated by the radiation moderation phenomenon is irradiated inside of a main chamber. The gases such as hydrogen, oxygen, carbon dioxide are supplied from the raw material supply unit 100 and passes through the inside of the main chamber, the quantum energy generated by the radiation moderation phenomenon is irradiated to the gases.

By quantum energy irradiation, the covalent bonds of the gases material are dissociated, and the gases material may become atoms, cations or anions.

A first ion generator 200A includes the first quantum energy generator 210 of the braking radiation type.

A second quantum energy generator 230 includes a sealed glass tube 237, a cathode 233, a first power supply unit 231 supplying direct current power to the cathode, an electron emission source 234, a gate electrode 243, a first anode 235, a second power supply unit 241 to apply high voltage to the first anode 235, a X-ray target plate 236 installed in the center of the right slope of the first anode 235, a second anode 253, a third power supply unit 251 to supply direct current high-voltage power to the second anode 253 and a quantum energy divergent layer 254 installed in contact with the surface of the second anode 233.

When the direct current power generated by the first power supply unit 231 is supplied to the cathode 233, the electron beams generated by the electron emission source 234 installed in contact with the cathode 233 are accelerated in the direction of the first anode 235 by the bias circuit configured between the first power supply unit 231 and the second power supply unit 241,
and the electron beams emitted and refracted X-rays by collision with the target surface 236 of the tungsten (W) material attached to the right slope of the first anode 235 is accelerated toward the second anode 253 by the bias circuit configured between the second power supply unit 241 and the third power supply unit 251.

And then the electron beams is reached the second anode 253 of rhodium, etc., and collided with the nuclear extraelectronic (extranuclear) of the anode material, a certain amount of kinetic energy is dissipated by the decay action, and the residual energy generates X-ray and quantum energy with waves. The X-ray and quantum energy penetrating the anode are converted into photoelectron and quantum energy with waves as they are irradiated to the emission layer 254 of non-radial matter, and the quantum energy generated by the radiation moderation phenomenon is irradiated inside of a main chamber. The gases such as hydrogen, oxygen, carbon dioxide are supplied from the raw material supply unit 100 and passes through the inside of the main chamber, the quantum energy generated by the radiation moderation phenomenon is irradiated to the gases.

By quantum energy irradiation, the covalent bonds of the gases material are dissociated, and the gases material may become atoms, cations or anions.

A second ion generator 200B includes the second quantum energy generator 230 of the braking radiation type,

A third quantum energy generator 270 of high-voltage discharge method includes a main body (a housing) 271, a first saddle gradient coil 272-1a having first discharge electrodes 272-2a, a second gradient saddle coil 272-1b having second discharge electrodes 272-2b, a modified Trigger coil 273 having a ground electrode 273a, a modified Trigger coil 274 having third discharge electrodes 274a, a high-voltage generator 278 and a conducting wire 279.

After insulating the inner surface of the main body 271 with an insulator, the first gradient coil 272-1a having the first discharge electrode 272-2a and the second gradient saddle coil 272-1b having the second discharge electrode 272-2b are installed in surface contact with the inner surface of the main body, and are installed to face each other at upper and lower intervals, and the wound coil directions are opposite to each other.

The modified Trigger coil 273 having the ground electrode 273a is installed at intervals from Gradient saddle coil 272-1a, 272-1b in the inner center direction.

The modified Trigger coil 274 having the third discharge electrode 274a is installed at intervals from the modified Trigger coil 273 in the inner center direction.

In the high-voltage generator 278 installed on the outside side, when the generated pulsed high-voltage is supplied to the first gradient saddle coil 272-1a having the first discharge electrodes 272-2a, the second gradient saddle coil 272-1b having the second discharge electrodes 272-2b, the modified Trigger coil 273 having the ground electrodes 273a, and the modified Trigger coil 274 having the third discharge electrodes 274a, between the coils, the electromagnetic field of the pulse form of the opposite direction is generated, and the electromagnetic fields of the pulse form of the opposite direction at the center distance are overlapped and offset (dissipated), and pulsating orient energy is generated in the state of the zero magnetic field, and it is irradiated inside the main body 271, and at the same time a high-voltage discharge is initiated between the coils 272, 273, 274.

The gases such as hydrogen, oxygen, carbon dioxide are supplied from the raw material supply unit 100 and passes through the inside of the main chamber, the quantum energy and the high-voltage discharge energy are irradiated to the gases.

By quantum energy irradiation and the high-voltage discharge energy, the covalent bonds of the gases material are dissociated, and the gases material may become atoms, cations or anions.

A third ion generator 200C includes the third quantum energy generator 270 of the high-voltage discharge method.

An ion generator 200 includes the first ion generator 200A, the second ion generator 200B and the third ion generator 200C.

A first dissolving device 310 includes a first circulating pump 311, a flow control valve 312, a venturi ejector 313, a pressure detection sensor 314, a main body (chamber) 315, a first defoamer 318 and a circulation tube 319.

The first defoamer 318 includes a first quantum energy generator consisting of a first quantum energy generating coil 316a, a second quantum energy generating coil 316b and a pulsed power supply unit 317.

By operating the first circulation pump 311, the solution stored in a main body 501 of a quantum energy transfer device 500A is sucked and pressurized, and hydrogen ions (H⁺, H⁻), oxygen ions (O⁺, O⁻) or nitrogen ions (N⁺, N⁻, NO) generated in the ion generator 200 are supplied in the process of passing through the venturi ejector 313.

Air bubbles mixed in the solution are primarily destroyed and decompressed during the process of passing through a 1 mm pore disperser 315a installed in the inner center of the main body 315 by supplying hydrogen ions (H⁺, H⁻), oxygen ions (O⁺, O⁻) or nitrogen ions (N⁺, N⁻, NO) into the main body 315.

In the pulse-type power supply unit 317 installed on the outside side, the generated pulsed high-voltage is supplied to the first and second quantum energy generating coils 316a, 316b of Uniform saddle coil.

The first and second quantum energy generating coils 316a, 316b are installed to face each other at interval, and the wound coil directions are opposite to each other.

Between the coils, the electromagnetic field of the pulse form of the opposite direction is generated, and the electromagnetic fields of the pulse form of the opposite direction at the center distance are overlapped and offset (dissipated), and a quantum energy is generated in the state of the zero magnetic field, and it is irradiated inside the main body 315.

The quantum energy irradiates to the solution inside the body 315 to depocalyse (destroy) the air bubbles contained in the solution, as the result, the hydrogen ions (H⁺, H⁻), oxygen ions (O⁺, O⁻) or nitrogen ions (N⁺, N⁻, NO) may be primary dissolved.

A second dissolving device 320 includes a second circulating pump 321, a flow control valve 322, a pressure detection sensor 323, a chamber 324, a second defoamer 327 and a circulation tube 324a.

The second defoamer 327 includes a second quantum energy generator consisting of a first quantum energy generating coil 325a, a second quantum energy generating coil 325b and a pulsed power supply unit 326.

When a control panel (600) supplies power to the second circulation pump 321, the second circulation pump 321 is operated so that the process fluid in which hydrogen ions (H⁺, H⁻), oxygen ions (O⁺, O⁻) or nitrogen ions (N⁺, N⁻, NO) are primarily dissolved in the first dissolving device 310 is sucked and pressurized by the second circulation pump 321 and introduced into the second defoamer 327 to which the second quantum energy is irradiated through the flow rate adjusting valve 322.

The chamber 324 of the second dissolving device 320 to which the second quantum energy is irradiated has a cylindrical shape having a predetermined diameter and a predetermined length, and is insulated from the inner surface of the cylindrical chamber 324 so that a first quantum energy generation coil 325a having Solenoid coil shape is installed to be in surface contact with the inner surface. A second quantum energy generation coil 325b having Trigger coil shape is installed at intervals, and a third quantum energy generation coil 325c having a modified RF coil shape is installed downward at intervals from the first and second quantum energy generation coils 325a and 325b.

Trigger voltage generator 326b is installed at one side of the outside of the main body, is connected to the pulsed power supply unit 326 with a conducting wire.

The first and second quantum energy generating coils 325a and 325b are connected to a pulsed power supply unit 326a through a conducting wire, and the third quantum energy generating coil 325c is connected to the Trigger voltage generator 326b through the conducting wire. The pulsed power supply unit 326a and the Trigger voltage generator 326b are also connected through a conducting wire.

The third quantum energy generation coil 325c starts discharging while pulse energy is transferred between the first and second quantum energy generation coils 325a and 325b having polarities different from those of the magnetic field. The third quantum energy generation coil 325c starts main discharge at a lower voltage than the high voltage pulse applied to the first quantum energy generation coil 325a and the second quantum energy generation coil 325b.

At the same time, when a high voltage pulsed power supply unit 326a generates a pulse-type high voltage to the first quantum energy generation coil 325a and the second quantum energy generation coil 325b installed such that winding directions of the coils are opposite to each other through a conducting wire, a pulse-type magnetic field in opposite directions is generated in the first quantum energy generation coil 325a and the second quantum energy generation coil 325b at an angle of 90° with respect to a flow direction of a current, pulse-type magnetic fields in opposite directions are overlapped and offset (dissipated) at a center distance of the first quantum energy generation coil 325a and the second quantum energy generation coil 325b, and quantum energy is generated in a zero magnetic field state and irradiated to the introduced fluid.

As a result, a shock wave is generated by the discharge, and at the same time, bubbles containing hydrogen ions (H⁺, H⁻), oxygen ions (O⁺, O⁻) or nitrogen ions (N⁺, N⁻, NO), which are not destroyed in the first dissolving device 310 passing through between the first quantum energy generation coil 325a, the second quantum energy generation coil 325b, and the third quantum energy generation coil 325c, are destroyed by the discharge shock wave, and the water hammer pressure generated by the destruction of the bubbles efficiently further destroys the bubbles adjacent to the discharge shock wave.

A first ion separator 400A includes a positive charge collector 410 and a first quantum energy generaton device 420. The positive charge collector 410 includes a main body 401, a first power supply unit 411, a coil 412 and multiple positive charge collecting electrodes 410. The main body 401 of the first ion separator 400A may have a cylindrical shape with conical upper and lower parts, or a rectangular parallelepiped shape with rectangular pyramid shapes at upper and lower parts. A plurality of first positive charge collecting electrodes 413 supplied with power generated by a power supply unit 411 of the positive charge collector 410 are installed in upper and lower holders 401a and 401b at intervals from each other inside the main body 401 and at intervals from the upper, lower, left, and right inner surfaces of the main body 401.

The positive charge collector 410 includes the power supply unit 411, the first coil 412, the positive charge collecting electrodes 413, and a conducting wire 414.

In addition, a first Cuffs coil 421 of the first quantum energy generator 420 is installed in outer upper area of the main body 401, a second Cuffs coil 422 is installed at in outer lower area of the main body 401, and a power supply unit 423 is installed at an interval to supply the power generated from the power supply unit 423 to the first Cuffs coil 421 and the second Cuffs coil 422 through a conducting wire 424.

Pulse-type variable power is supplied to the first Cuffs coil 421 and the second Cuffs coil 422 wound in opposite directions through a wire from the power supply unit 423 of the first quantum energy generator 420. In the first Cuffs coil 421, a pulsed electromagnetic field (PEMF) is irradiated in the lower direction of the main body 401 at an angle of 90 degrees with the current flow direction, and in the second Cuffs coil 422, a pulsed electromagnetic field (PEMF) is irradiated in the upper direction of the main body 401 at an angle of 90 degrees with the current flow direction.

As a result, the pulse-type magnetic fields irradiated in opposite directions at the central distance between the first Cuffs coil 421 and the second Cuffs coil 422 overlap and offset (dissipate), and the pulsation quantum energy generated in the zero magnetic field state is irradiated into the central distance space between the first Cuffs coil 421 and the second Cuffs coil 422 inside the main body 401.

In the positive charge collector 410, the output positive (+) terminal of the power supply unit 411 is directly connected to the first coil 412 through a conducting wire, but the output negative (−) terminal of the power supply unit 411 is connected to the plurality of first positive charge collecting electrodes 413a, 413c, and 413e through a conducting wire, and each of second positive charge collecting electrodes 413b, 413d, and 413f is separated from the first positive charge collecting electrodes 413a, 413c, and 413e at an interval so that current does not flow.

The first coil 412 is directly supplied with positive (+) power of the power supply unit 411, and negative (−) power is not supplied to the first coil 412 installed to be connected to the second positive charge collecting electrodes 413b, 413d, and 413f installed separately from the first positive charge collecting electrodes 413a, 413c, and 413f connected to the output negative (−) terminal of the power supply unit 411. Therefore, between the first positive charge collecting electrodes 413a, 413c, and 413e and the second positive charge collecting electrodes 413b, 413d, and 413f, there is a strong attraction to supply negative (−) power.

As a result, hydrogen ions (H⁺, H⁻), oxygen ions (O⁺, O⁻) or nitrogen ions (N⁺, N⁻, NO) generated by dissociating hydrogen molecules, oxygen molecules or nitrogen molecules in the ion generators 200 are introduced into the first ion separator 400A and pass through between the first and second collection electrodes, and hydrogen ions (H⁺), oxygen ions (O⁺) or nitrogen ions (N⁺) are adsorbed (captured) to the first charge collecting electrodes 413a, 413c, and 413e through which negative (−) currents flow and the second charge collecting electrodes 413b, 413d, and 413f in which attraction acts. On the contrary, hydride ions (H⁻), oxygen ions (O⁻) or nitrogen ions (N⁻) may be supplied to the energy transfer device 500 by the pressing force of the second circulation pump 321 by acting a repulsive force with the first charge collecting electrodes 413a, 413c, and 413e through which negative (−) current flows.

The second ion separator 400B includes a negative charge collector 430 and a second quantum energy generaton device 440. The negative charge collector 430 includes a main body 402, a first power supply unit 431, a coil 432 and multiple negative charge collecting electrodes 433. And the second quantum energy generaton device 440 includes a first Cuffs coil 441, a second Cuffs coil 442 and a power supply unit 443. The second ion separator 400B includes a negative charge collector 430 and a second quantum energy generaton device 440. The negative charge collector 430 includes a main body 402, a first power supply unit 431, a coil 432 and multiple negative charge collecting electrodes 433. And the second quantum energy generaton device 440 includes a first Cuffs coil 441, a second Cuffs coil 442 and a power supply unit 443. The main body 402 of the second ion separator 400B may have a cylindrical shape with conical upper and lower parts, or a rectangular parallelepiped shape with rectangular pyramid shapes at upper and lower parts.

The plurality of first negative charge collecting electrodes 433 supplied with power generated by the power supply unit 431 of the negative charge collector 430 are installed in the upper and lower holders at intervals from each other inside the main body 402 and at intervals from the upper, lower, left, and right inner surfaces of the main body 402.

In addition, the first Cuffs coil 441 of the second quantum energy generator 440 is installed in the outer upper area of the main body 402, the second Cuffs coil 442 is installed in the outer lower area of the main body 402, and the power supply unit 443 is installed at an interval to supply the power generated from the power supply unit 443 to the first Cuffs coil 441 and the second Cuffs coil 442 through the conducting wire.

Pulse-type variable power is supplied to the first Cuffs coil 441 and the second Cuffs coil 442 wound in opposite directions through a wire from the power supply unit 443 of the second quantum energy generator 440. In the first Cuffs coil 441, a pulsed electromagnetic field (PEMF) is irradiated in the lower direction of the main body 402 at an angle of 90 degrees with the current flow direction, and in the second Cuffs coil 442, a pulsed electromagnetic field (PEMF) is irradiated in the upper direction of the main body 402 at an angle of 90 degrees with the current flow direction.

As a result, the pulse-type magnetic fields irradiated in opposite directions at the central distance between the first Cuffs coil 441 and the second Cuffs coil 442 overlap and offset (dissipate), and the pulsation quantum energy generated in the zero magnetic field state is irradiated into the central distance space between the first Cuffs coil 441 and the second Cuffs coil 442 inside the main body 402.

In the negative charge collector 430, the output negative (−) terminal of the power supply unit 431 is directly connected to the second coil 432 through a conducting wire, but the output positive (+) terminal of the power supply unit 431 is connected to a plurality of first charge collecting electrodes 433a, 433c, and 433e through a conducting wire, and each of second charge collecting electrodes 433b, 433d, and 433f is separated from the first charge collecting electrodes 433a, 433c, and 433e at an interval so that current does not flow.

The second coil 432 is directly supplied with negative (−) power of the power supply unit 431, and positive (+) power is not supplied to the second coil 432 installed to be connected to the second charge collecting electrodes 433b, 433d, and 433f installed separately from the first charge collecting electrodes 433a, 433c, and 433f connected to the output positive (+) terminal of the power supply unit 431. Therefore, between the first charge collecting electrodes 433a, 433c, and 433e and the second charge collecting electrodes 433b, 433d, and 433f, there is a strong attraction to supply positive (+) power.

As a result, hydrogen ions (H⁺, H⁻), oxygen ions (O⁺, O⁻) or nitrogen ions (N⁺, N⁻, NO) generated by dissociating hydrogen molecules, oxygen molecules or nitrogen molecules in the first, second, and third ion generators 200A, 200B, and 200C are introduced into the first and second ion separators 400A and 400B and pass through between the first and second collection electrodes, and hydrogen ions (H⁻), oxygen ions (O⁻) or nitrogen ions (N⁻) are adsorbed (captured) to the first charge collecting electrodes 433a, 433c, and 433e through which positive (+) currents flow and the second charge collecting electrodes 433b, 433d, and 433f in which attraction acts.

A third ion separator 400C includes a reactor 440, a condenser 450 and a positive charge collector 457. The reactor 440 includes a main body 403, a jacket 403a, a cooling water circulation line 444a, a hot water circulation line 444b, a municipal water or purified water supply line 445, a raw material input line 446, and a stirrer 447. The lower part of the main body 403 of the reactor 440 is conical, the upper part of the main body 403 is inclined, and the main body 403 may have a dual-structure cylindrical shape including a jacket 403a. The cooling water supply and exhaust line 444a, and the hot water supply and exhaust line 444b may be connected and installed on one side of the side lower portion and one side of the side upper portion of the jacket 403a.

The condenser 450 includes a main body 451, a cooling water circulation line 451a, and a exhaust line 452 of hydrogen ions (H⁻) or oxygen ions (O⁻).

The positive charge collector 457 includes a plurality of first cathodes (453a, 453b, 453c, 453d), a first coil (454; the load), a second power supply unit 455, and conducting wires 456.

The electronic valve 445a of the municipal water or the purified water supply line 445 is opened and the municipal water or the purified water is filled with 60 to 75% of the volume of the main body 403 of the container 440. The electronic valve of the hot water supply and exhaust line 444a is opened, and the hot water are circulated from the lower supply line of the jacket 403a to the top exhaust line 444a and heat the municipal water or the purified water filled with appropriate amount inside the main body 403 to 40 to 60 degrees Celsius. The mixer 447 is operated, and the mixer 447 stirs the municipal water or the purified water in the range of 30 to 60 RPM. The pulse-type DC power generated by the power supply unit 443 is supplied to the first electrode 441 and the second electrode 442 installed on the inner side surface of the reactor 440.

The cover of the hopper 446 is opened, and the metal hydrate in which one kind material is selected from the lithium hydride (LiH), the sodium hydride (NaH) and the potassium hydride (KH) is supplied to the gravity difference to the main body 403. Hydride ions (H⁻) may be generated by a hydrolysis reaction and an electrolysis reaction between the metal hydrate and the water. As the pulse-type DC power generated by the first power supply unit 443 is supplied to the first electrode 441 and the second electrode 442, the metal hydrate introduced into the main body 403 is subjected to an electrolysis reaction to generate a hydrogen ions (H⁻).

Hydride ions (H⁻) generated by the hydration reaction flow into the main body 451 of the condenser 450 of a shell and tube shape through the supply line 452 and are cooled by indirect heat exchange with cooling water introduced into the cooling water supply line and the exhaust line 451 installed on one side of the lower and upper sides of the body 451 to condense and liquefy the water vapor of water molecules mixed with hydride ions (H⁻).

The liquefied water is discharged to the outside through a condensate exhaust line installed under the left cell 451a of the main body 451 so that hydride ions (H⁻) and water vapor are separated and introduced into the quantum energy transfer devices. In this process, positive charges may be collected on the first and second cathodes to suppress a reaction between hydride ions (H⁻) and a small amount of lithium ions (Li⁺) or sodium ions (Na⁺) and potassium ions (K⁺).

Specifically, negative (−) terminals on the output side of the power supply unit 455 of the positive charge collector 457 are connected to each of the plurality of first negative electrodes 453a-1, 453b-1, 453c-1, and 453d-1 of the positive charge collector, positive (+) terminals are connected to the first coil 454, and another electric wire of the first coil 454 is connected to the second negative electrodes 453a-2, 453b-2, 453c-2, and 453c-2. The second negative electrodes 453a-2, 453b-2, 453c-2, and 453c-2 are installed to be separated from the first negative electrodes 453a-1, 453b-1, 453c-1, and 453d-1.

Negative (−) power is supplied to the first negative electrodes 453a-1, 453b-1, 453c-1, and 453d-1, and no power is supplied to the second negative electrodes 453a-2, 453b-2, 453c-2, and 453d-2, therefore, between the first negative electrodes 453a-1, 453b-1, 453c-1, and 453d-1 and the second negative electrodes 453a-2, 453b-2, 453c-2, and 453d-2, there is a strong attraction to supply negative (−) power.

Therefore, lithium ions (Li⁺), sodium ions (Na⁺), and potassium ions (K⁺) are collected in the first and second cathodes inside the condenser, and hydrogen ions (H⁻) are separated from the cations by repulsive force and introduced into the quantum energy transfer devices by the pressing force of the air FAN.

A first quantum energy transfer device 500A includes an ion gas supply line 501a, a municipal water or purified water supply line 501b, a solution supply line 501c in which ions are dissolved, a circulation and exhaust line 505a and 505b, a first main body 501, a second main body 502, a stirrer 503, a circulation pump 504, a first quantum energy generation device 510, and a second quantum energy generation device 520. The first quantum energy generation device 510 includes a first quantum energy generation coil 511, a second quantum energy generation coil 512, a first power supply unit 513 for supplying power in a pulse form, and a conducting wire 514. The second quantum energy generation device 520 includes a third quantum energy generation coil 521, a fourth quantum energy generation coil 522, a second power supply unit 523 for supplying power in a pulse form, and a conducting wire 524.

A solution containing hydrogen ions (H⁺, H⁻) or oxygen ions (O⁺, O⁻) in the dissolving device 300 is sent to the supply line 501c by the pressing force of the pressure pump 321, followed by 70-80% of the internal volume of the main body 501 through the venturi ejector 506.

Alternatively, a first solution in which hydrogen ions (H⁻) or oxygen ions (O⁻) supplied from the first and third ion separators 400A and 400C, or hydrogen ions (H⁺) or oxygen ions (O⁺) supplied from the secondary ion separator 400B are mixed with purified water (municipal water) may be filled to 70 to 80% of the internal volume of the main body 501 through the supply line 501B and the venturi ejector 506.

Alternatively, a second solution in which hydrogen ions (H⁻) or oxygen ions (O⁻) supplied from the first and third ion separators 400A and 400C, or hydrogen ions (H⁻) or oxygen ions (O—) supplied from the secondary ion separator 400B are mixed with purified water (municipal water) may be filled to 70 to 80% of the internal volume of the main body 501 through the supply line 501B and the venturi ejector 506.

In this process, the level sensor detects the water level and transmits it to the control panel 600, and the control panel 600 stops the pressure pump 321 and supplies power to the stirrer 501 to rotate the first solution or the second solution supplied to the main body 501.

At the same time, when pulsed direct current power is supplied from the first power supply 513 to the first and second quantum energy generating coils 511 and 512 wound in opposite directions through a wire, an electromagnetic field in the form of a pulse is generated at an angle of 90 degrees in the current flow direction and transferred to the first solution or the first solution.

As a result, electromagnetic fields in the form of pulses in opposite directions overlap and offset (dissipate) in the center of the first body 501, generating quantum energy in a zero magnetic field state, and the quantum energy above can be irradiated to the solution.

Subsequently, when power is supplied to the circulation pump 504 from the control panel 600, the solution irradiated with electromagnetic fields and quantum energy is sucked and pressurized inside the first body 501 and supplied to the second body 502.

The third quantum energy generating coils 521 and the fourth quantum energy generating coils 522 may be wound in opposite directions, and an inlet and an outlet hole may be perforated between the coil and the coil wound with a predetermined winding number.

The pulse-type power generated by the second power supply unit 513 is applied to the third quantum energy generating coils 521, in which the coils are wound in opposite directions, and the pulse-type electromagnetic field generated at an angle of 90 degrees in the current flow direction is installed to face each other. In addition, the opposite pulse-type electromagnetic energy generated between the third quantum energy generating coils 521 and the fourth quantum energy generating coils 522 may be irradiated to the solution passing through the inlet and outlet holes. The electromagnetic energy in the form of pulses in opposite directions overlap and offset (dissipate), and the pulse-type quantum energy generated in the zero magnetic field state is irradiated and continues to circulate for a certain period of time, before the electric valve 505a installed on the circulation line 505 above the second body 502 is closed and the electric valve 505b is opened to supply the solution to which the electromagnetic field and quantum energy are irradiated.

A second quantum energy transfer device 500B includes a main body 503, an ion gas supply line 504, a first quantum energy generation device 530, an upper first rotating body motor 544, a pneumatic cylinder 544a, a first rotating disk 544b, a lower second rotating body motor 545, a second rotating disk 545b, and a second quantum energy generation device 540. The first quantum energy generation device 530 includes a first quantum energy generation coil 531, a second quantum energy generation coil 532, a first power supply unit 533, and a conducting wire 534. The second quantum energy generation device 540 includes a third quantum energy generation coil 541, a fourth quantum energy generation coil 542, a pulse-type power supply unit 543, and a conducting wire.

When the control panel 600 supplies power to the motor 545a for the second rotating body 545, the motor 544a for the first rotating body 544, and the pneumatic or hydraulic cylinder 546, the motor 545a of the second rotating body 545 is operated to rotate the motor shaft and the second rotating disk 545c. At the same time, the pneumatic or hydraulic cylinder 546 installed at the center of the upper portion of the main body 503 is operated to lower the first rotating body 544 composed of the motor 544a, the shaft 544b, and the turntable 544c to the close distance (10 to 20 cm) of the quantum energy treatment object loaded on the turntable 545c of the second rotating body 545 inside the main body 503,
and then the motor 544a of the first rotating body 544 is operated in the control panel to rotate the shaft 544b connected to the motor shaft and the turntable 544c to rotate the hydrogen ion (H⁻) or oxygen ions (O⁻) supplied from the first ion separator 400A, or oxygen ions (O⁺), the hydrogen ion (H⁺), the nitrogen ions (N⁺) or the nitrogen oxide ions (NO) supplied from the second ion separator 400B in a state where the ions are supplied or not supplied to one side of the upper portion of the main body 503 of the second quantum energy transfer device 500B by the pressing force of the air FAN 157. At the same time, the pulse-shaped electromagnetic fields generated by the first power supply unit 533 are irradiated from the upper portion to the lower portion and from the lower portion to the upper portion to the loaded quantum energy transfer target in the first quantum energy generation coil 531 of the plurality of first quantum energy generators installed to be in surface contact with the rear surface of the turntable 544c of the first rotating body 544 and the second quantum energy generation coil 532 installed to be in surface contact with the rear surface of the turntable 545c of the second rotating body 545 so that the pulse-shaped electromagnetic fields generated by the first power supply unit 533 are overlapped and offset (dissipated) in the center direction of the loaded target and transferred to the loaded object.

A third quantum energy transfer device 500C includes a main body 504, a screw driving motor 505, a screw 506, a powder inlet 507, a powder outlet 507a, a supply fan 508, an ion gas supply line 509 and a third quantum energy generation device 550C. The third quantum energy generation device 550C includes a plurality of first quantum energy generation coils 551 (551a˜551j), a plurality of second quantum energy generation coils 552 (552a˜552j), a first power supply unit 553, and a conducting wire 554.

Hydrogen ions (H⁻), oxygen ions (O⁻), nitrogen ions (N⁻), nitrogen oxide ions (NO), hydrogen ions (H⁺), oxygen ions (O⁺) or nitrogen ions (N⁺) supplied from the ion separators (400A, 400B, 400C), may be supplied to the venturi neck part (powder outlet) 507a connected to the supply FAN 508 of the third quantum energy transfer device 500C by the suction power of the supply FAN 509 or by the pressing force of the air FAN 137.

At the same time, when the quantum processing object (powder) is introduced into the main body 504 by the blade of the supply FAN 508, and discharged to the outlet 507a by the screw 506 driven by the drive motor 505′, the pulse-type power generated by the pulse-type power supply unit 553 is supplied through the wire to the multiple first quantum energy generating coils 551 and second quantum energy generating coils 552 installed to face each other in the circumferential direction of the outer circumferential surface of the main body 504, an electromagnetic field in the form of a pulse generated at a 90 degree angle in the current flow direction is irradiated from the top to the bottom of the main body 504 in the first quantum energy generating coil 551, and the second quantum energy generating coil 552 is irradiated from the bottom of the main body 504 to the top of the main body 504, and the pulse-type electromagnetic fields in opposite directions overlap and offset (dissipate) in the screw shaft portion to generate quantum energy in a zero magnetic field state.

When oxygen ions (O⁺, O⁻) are supplied from the ion gas supply line 509 while the generated quantum energy is irradiated to the quantum energy treatment object, the powder raw material may be mixed with oxygen ions (O⁺, O⁻) by screw driving inside the main body 504 to be perform an oxidation reaction.

When hydride ions (H⁻) or hydrogen ions (H⁺) are supplied from the ion gas supply line 509, the powder raw material may be mixed with hydride ions (H⁻) or hydrogen ions (H⁺) by screw driving inside the main body 504 to be perform a reduction reaction.

When nitrogen ions (N⁺, N⁻, NO) is supplied from the ion gas supply line 509, the powder raw material may be mixed with the nitrogen ions (N⁺, N⁻, NO) by screw driving inside the main body 504 to be perform a nitrification reaction.

### Advantageous Effects

According to the present invention, devices that mixes ionized hydrogen ions or oxygen ions with gas, liquid, or solid substances and transfers them to a mixture have the following effects.
1) A large amount of hydrogen ions (H⁺) or hydride ions (H⁻) can be produced without the risk of fire or explosion by mixing more than 96% of nitrogen, carbon dioxide gas, or argon gas with hydrogen gas supplied from a high-pressure container or produced by hydrolysis.
2) A large amount of oxygen ions (O⁺, O⁻) can be produced by irradiating high-voltage discharge and braking radial quantum energy to oxygen gas supplied from a high-pressure container or generated by hydrolysis.
3) A large amount of oxygen ions (O⁺, O⁻) and nitrogen ions (N⁺, N⁻, NO) can be produced by irradiating high-voltage discharge and braking radial quantum energy to nitrogen gas and oxygen gas in the air supplied by an air FAN.
4) It can be easily separated into positive (+) ions and negative (−) ions by supplying power of different polarities from the ionization separation device.
5) Quantum energy in the form of pulsations in various frequency bands can be transferred to gaseous, liquid and solid-phase materials.

### DESCRIPTION OF DRAWINGS

FIG. 1 is a schematic diagram illustrating the overall configuration of apparatus for mixing ionized hydrogen and oxygen ions with gas-phase, liquid-phase, and solid-phase substances and transferring quantum energy to mixtures according to the present invention.

FIG. 2 is a cross-sectional view illustrating the raw material supply unit shown in FIG. 1.

FIG. 3 is a schematic diagram illustrating a third raw material supply unit 130 unit of the raw material supply unit 100 of FIG. 1.

FIG. 4 is a schematic diagram illustrating the ion generator shown in FIG. 1.

FIG. 5A is a cross-sectional view illustrating a first ion generator 200A of FIG. 4.

FIG. 5B is a cross-sectional view illustrating a second ion generator 200B of FIG. 4.

FIG. 5C is a cross-sectional view illustrating a third ion generator 200C of FIG. 4.

FIG. 6 is a cross-sectional view illustrating a dissolving device 300.

FIG. 7 is a schematic drawing illustrating the first and second ions separators 400A and 400B of FIG. 1.

FIG. 8 is a schematic drawing illustrating the third ion separator 400C of FIG. 1.

FIG. 9 is a schematic drawing illustrating the first quantum energy transfer device 500A of FIG. 1.

FIG. 10 is a schematic drawing illustrating the second quantum energy transfer device 500B of FIG. 1.

FIG. 11 is a schematic drawing illustrating the third quantum energy transfer device 500C of FIG. 1.

FIG. 12 is a schematic drawing illustrating a fourth quantum energy transfer device 500D of FIG. 1.

FIG. 13a is a schematic drawing illustrating a shape of first and second quantum energy generating coils implemented in a shape of Cuffs coil.

FIG. 13b is a schematic drawing illustrating a shape of the energy generating coils (the first and second energy generating coils) formed in Helmhelz coil shape.

FIG. 13c is a schematic drawing illustrating a shape of a quantum energy generating coil (first and second quantum energy generating coils) implemented in Gradient saddle coil shape.

FIG. 13d is schematic drawings illustrating the shapes of the quantum energy generating coils (first and second quantum energy generating coils) implemented in the shape of Gradient saddle coil and Uniform saddle coil.

FIG. 13e is a schematic drawing illustrating a shape of quantum energy generation coils (first and second quantum energy generation coils) implemented in a shape of Toroidal coil.

FIG. 13f is a schematic drawing illustrating a shape of a quantum energy generating coil (first and second quantum energy generating coils) implemented in Trigger coil shape.

FIG. 13g is a schematic drawing illustrating a shape of Zigzag-shaped quantum energy generating coil (first and second quantum energy generating coils).

FIG. 13h is a schematic drawing illustrating a shape of a quantum energy generating coil (first and second quantum energy generating coils) implemented in Temple-shaped coil.

FIG. 13i is a schematic drawing illustrating a shape of a quantum energy generation coil (first and second quantum energy generation coils) implemented in a motor stator-type coil shape.

FIG. 13j is a schematic drawing illustrating a shape of a quantum energy generating coil (first and second quantum energy generating coils) implemented in a straight-angle shape coil or an edge-wise voice shape coil.

FIG. 13k is a schematic drawing illustrating a shape of a quantum energy generating coil (first and second quantum energy generating coils) implemented in Toroidal coil shape.

FIG. 131 is a schematic drawing illustrating a shape of a quantum energy generation coil (first and second quantum energy generation coils) implemented in Solenoid coil shape.

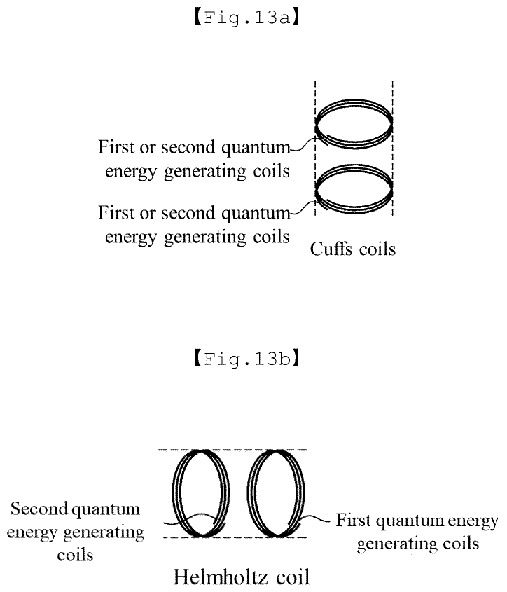

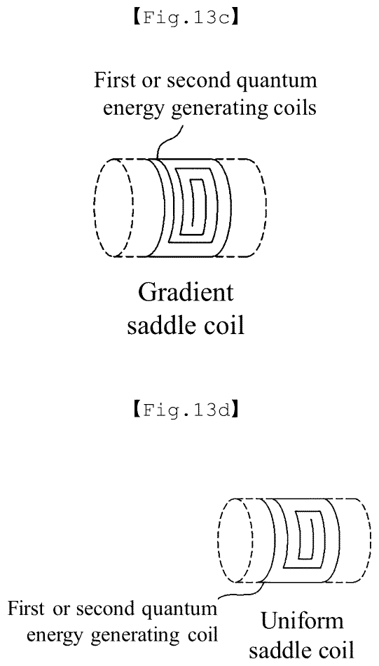

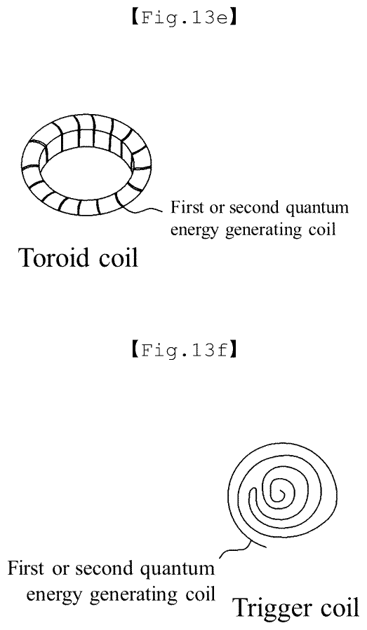

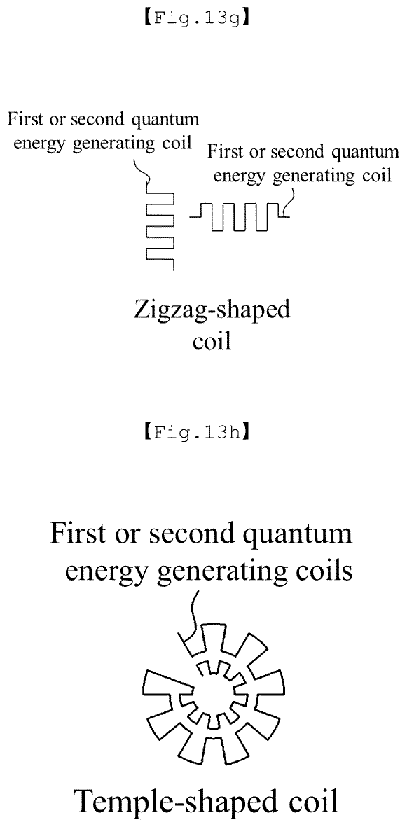

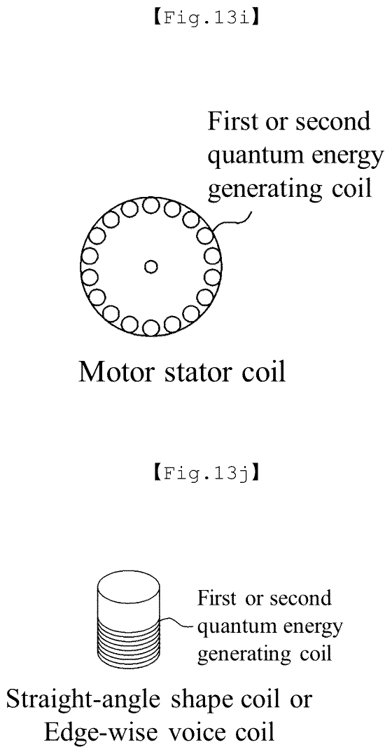

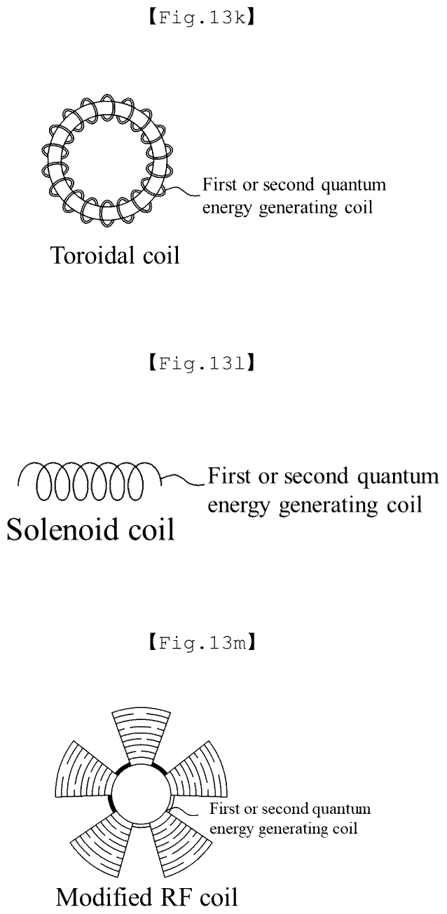

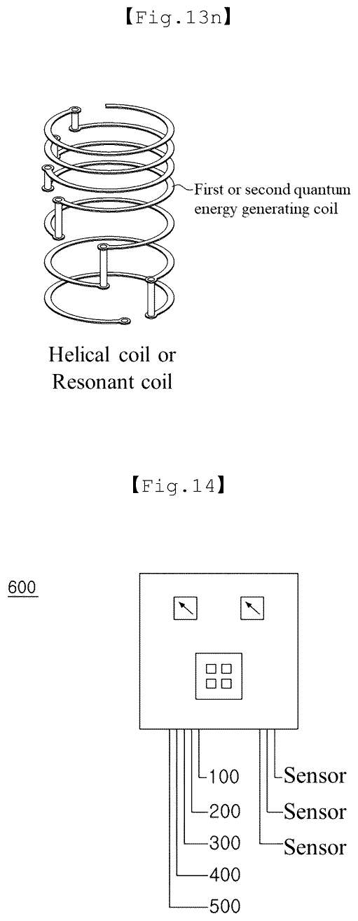

FIG. 13m is a schematic drawing illustrating shapes of quantum energy generating coils (first and second quantum energy generating coils) implemented in a modified RFcoil shape.

FIG. 13n is a schematic drawing illustrating a shape of a quantum energy generating coil (first and second quantum energy generating coils) implemented in Helical or Resonant coil shape.

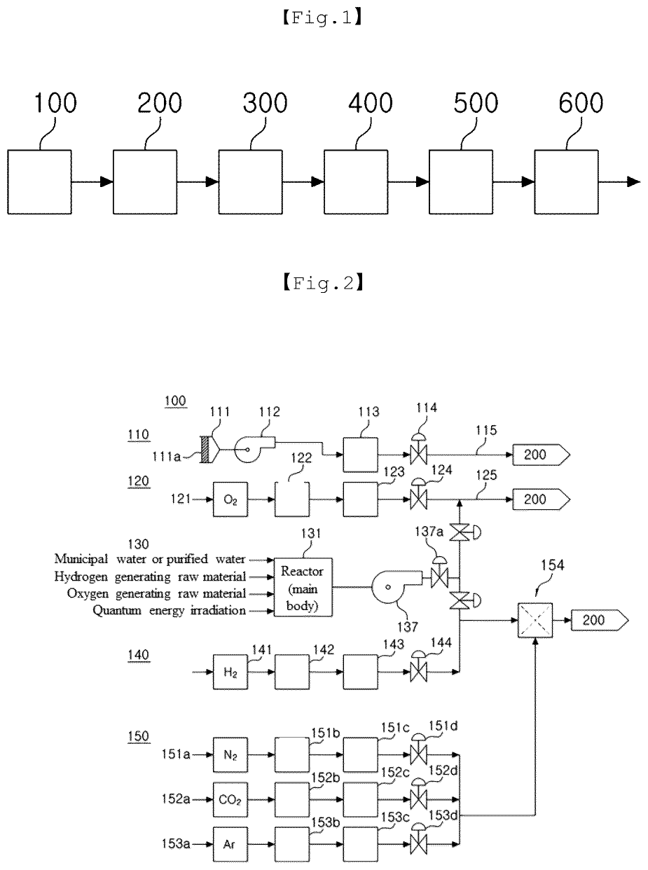

FIG. 14 is a schematic diagram illustrating a control panel of quantum energy transfer device in FIG. 1.

### BEST MODE FOR INVENTION

The best embodiment of this invention is presented in claim 1, and apparatus for mixing ionized hydrogen and oxygen ions with gas-phase, liquid-phase, and solid-phase substances and transferring quantum energy to mixtures is presented.

### MODE FOR INVENTION

A first raw material supply unit 110 includes a filter housing supply fan 112, a flow regulator 113, an electronic valve 114, and an air supply line 115, and the supply fan 112 is operated to suck in external air. In the process, the HEPA filter (not shown) mounted inside the filter housing 112 filters fine dust and adjusts the air supply flow rate from the flow regulator 113 to the appropriate flow rate and supplies through the supply line 115 to the ion generator 200; and
A second raw material supply unit 120 includes a pressure regulator 122, a flow regulator 123, an electronic valve 124, and an air supply line 125, and oxygen gas is supplied to the pressure regulator 122 by opening the main valve (not shown) of the container filled at high pressure (120 kg/cm²), depressurizing to the range of 50 mm Aq˜350 mm Aq in the pressure regulator 122, and supplying to the flow regulator 123, and after adjusting the flow rate at an appropriate flow rate in the flow regulator 123, supplied to the supply line 125 through the electronic valve 124 and supplied to the ion generator 200;
A third raw material supply unit 130 includes a 3-1 raw material supply unit 130a and a 3-2 raw material supply unit 130b.

The 3-1 raw material supply unit (130a) includes a main body 131, a stirrer 132, a motor 132a, a first bevel gear 132b, a second bevel gear 132c, a rotating shaft 132d, a first quantum energy generation coil 133, a second quantum energy generation coil 134, a power supply unit 135, storage tanks 136a and 136b, an exhaust fan 137, a municipal water or purified water supply line 138 and an exhaust line 139, and the storage tanks 136a and 136b store hydrogen generating raw materials such as borohydride salt (MBH₄) and triborohydride salt (MB₃H₈).

The first quantum energy generating coil 133 is installed on the outer circumferential surface of the rotating shaft 132d of the stirrer, and the second quantum energy generating coil 134 is installed on the inner circumferential surface of the main body 131.

While stirring the municipal water or purified water stored in the main body 131 by operating the stirrer 132, pulse-shaped power generated by the power supply unit 135 is supplied to the first and the second quantum energy generation coil 134.

As a result, the pulse-shaped electromagnetic field and the electromagnetic field in the opposite direction overlap and offset (dissipate) to produce pulsating quantum energy generated in a zero magnetic field state.

While the pulsating quantum energy is irradiating municipal water or purified water, the hydrogen generating raw materials stored in the storage tanks 136a, 136b are supplied, and the hydrogen generated by the hydrolysis reaction with municipal water or purified water is supplied to a mixer 154 using the exhaust fan 137.

The 3-2 raw material supply unit 130b includes a main body 131, a stirrer 132, a motor 132a, a first bevel gear 132b, a second bevel gear 132c, a rotating shaft 132d, a first quantum energy generation coil 133, a second quantum energy generation coil 134, a power supply unit 135, storage tanks 136c and 136d, an exhaust fan 137, a municipal water or purified water supply line 138 and an exhaust line 139.

The storage tank 136c is stored oxygen generating raw materials such as sodium peroxide and potassium peroxide, and the storage tank 136d is stored catalyst materials such as ferric formate, ferric citrate and ferric ammonium citrate.

The first quantum energy generating coil 133 is installed on the outer circumferential surface of the rotating shaft 132d of the stirrer, and the second quantum energy generating coil 134 is installed on the inner circumferential surface of the main body 131.

While stirring the municipal water or purified water stored in the main body 131 by operating the stirrer 132, pulse-shaped power generated by the power supply unit 135 is supplied to the first and the second quantum energy generation coil 134.

As a result, the pulse-shaped electromagnetic field and the electromagnetic field in the opposite direction overlap and offset (dissipate) to produce pulsating quantum energy generated in a zero magnetic field state.

While the pulsating quantum energy is irradiating municipal water or purified water, the oxygen generating raw materials and catalyst materials stored in the storage tanks 136c, 136d are supplied, and the oxygen generated by the hydrolysis reaction with municipal water or purified water is supplied to an ion generator 200 using the exhaust fan 137.

A fourth raw material supply unit 140 includes a container 141 filled with hydrogen gas, a pressure regulator 142, a flow regulator 143 and an electronic valve 144. Hydrogen gas (H₂) is filled in container 141 with high pressure (120 kg/cm²), the main valve (not shown) of the container 141 is opened and supplied to the pressure regulator 142, and supplied to the flow regulator 143 after depressurization in the range of 50 mm Aq˜350 mmAq, and after adjusting the flow rate to an appropriate flow rate in the flow regulator 143, it is supplied to the mixer 154 through the supply line and mixed with the inert gas and then supplied to the ion generator 200.

A first inert gas supply unit 151 includes a container 151a filled with nitrogen gas, a pressure regulator 151b, a flow regulator 151c and an electronic valve 151d. Nitrogen gas (N₂) is supplied to the pressure regulator 151b after opening the main valve (not shown) of the container 151a filled with high pressure (120 kg/cm²), and after depressurizing the pressure regulator 151b to the range of 50 mm Aq˜350 mmAq, supplying it to the flow regulator 151 c, and after adjusting the flow rate to the appropriate flow rate in the flow regulator 151c, it is supplied to the mixer 154 through the supply line.

A second inert gas supply unit 152 includes a container 152a filled with carbon dioxide gas, a pressure regulator 152b, a flow regulator 152c and an electronic valve 152d. Carbon dioxide gas (CO₂) is supplied to the pressure regulator 152b after opening the main valve of the container 152a filled with high pressure (120 kg/cm²), and after depressurizing the pressure regulator 152b to the range of 50 mm Aq˜350 mmAq, supplying it to the flow regulator 152c, and after adjusting the flow rate to the appropriate flow rate in the flow regulator 152c, it is supplied to the mixer 154 through the supply line.

A third inert gas supply unit 153 includes a container 153a filled with argon gas, a pressure regulator 153b, a flow regulator 153c and an electronic valve 153d. Argon gas (Ar) is supplied to the pressure regulator 153b after opening the main valve of the container 153a filled with high pressure (120 kg/cm²), and after depressurizing the pressure regulator 153b to the range of 50 mm Aq˜350 mmAq, supplying it to the flow regulator 153c, and after adjusting the flow rate to the appropriate flow rate in the flow regulator 153c, it is supplied to the mixer 154 through the supply line.

An inert gas supply unit 150 includes the first inert gas supply unit 151, the second inert gas supply unit 152 and the third inert gas supply unit 153.

A raw material supply unit 100 includes the first raw material supply unit 110, the second raw material supply unit 120, the third raw material supply unit 130, the fourth raw material supply unit 140 and the inert gas supply unit 150,

A first quantum energy generator 210 includes a sealed glass tube 201, a thermal electron emission cathode 204, a first power supply unit 202 supplying direct current power to the cathode, a first anode 213, a second power supply unit 211 to apply high voltage to the first anode 213, a target plate 213a installed in contact with the first anode 213, a second anode 223, a third power supply unit 221 to supply direct current high-voltage power to the second anode 223 and a quantum energy divergent layer 224.

The direct current power generated by the first power supply unit 202 is supplied and heated to the thermal electron emission cathode 204, and then thermal electrons are emitted from the thermal electron emission cathode 204.

The high voltage generated by the second power supply unit 211 is supplied to the first anode 213, and then the thermal electrons emitted from the negative electrode 204 by a bias circuit configured between the first power supply unit 202 and the second power supply unit 211 accelerates in the direction of the first anode 213 and collides with the target plate 213a and refracts at a 90 degree angle downward.

The high voltage generated by the third power supply unit 221 is supplied to the second anode 223, and then the thermal electrons collided with the target plate 213a accelerates in the direction of the second anode 223 by a bias circuit configured between the second power supply unit 211 and the third power supply unit 221, after being impacted by the collision with the extranucleus electrons of the second anode 223 material, a certain amount of kinetic energy is dissipated by the damping action, and the residual energy generates X-ray and quantum energy with waves.

The X-ray and quantum energy penetrating the anode are converted into photoelectron and quantum energy with waves as they are irradiated to the emission layer 224 of non-radial matter, and the quantum energy generated by the radiation moderation phenomenon is irradiated inside of a main chamber,

The gases such as hydrogen, oxygen, carbon dioxide are supplied from the raw material supply unit 100 and passes through the inside of the main chamber, the quantum energy generated by the radiation moderation phenomenon is irradiated to the gases. By quantum energy irradiation, the covalent bonds of the gases material are dissociated, and the gases material may become atoms, cations or anions.

A first ion generator 200A includes the first quantum energy generator 210 of the braking radiation type.

A second quantum energy generator 230 includes a sealed glass tube 237, a cathode 233, a first power supply unit 231 supplying direct current power to the cathode, an electron emission source 234, a gate electrode 243, a first anode 235, a second power supply unit 241 to apply high voltage to the first anode 235, a X-ray target plate 236 installed in the center of the right slope of the first anode 235, a second anode 253, a third power supply unit 251 to supply direct current high-voltage power to the second anode 253 and a quantum energy divergent layer 254 installed in contact with the surface of the second anode 233.

When the direct current power generated by the first power supply unit 231 is supplied to the cathode 233, the electron beams generated by the electron emission source 234 installed in contact with the cathode 233 are accelerated in the direction of the first anode 235 by the bias circuit configured between the first power supply unit 231 and the second power supply unit 241,
and the electron beams emitted and refracted X-rays by collision with the target surface 236 of the tungsten (W) material attached to the right slope of the t anode 235 is accelerated toward the second anode 253 by the bias circuit configured between the second power supply unit 241 and the third power supply unit 251.

And then the electron beams is reached the second anode 253 of rhodium, etc., and collided with the nuclear extraelectronic (extranuclear) of the anode material, a certain amount of kinetic energy is dissipated by the decay action, and the residual energy generates X-ray and quantum energy with waves. The X-ray and quantum energy penetrating the anode are converted into photoelectron and quantum energy with waves as they are irradiated to the emission layer 254 of non-radial matter, and the quantum energy generated by the radiation moderation phenomenon is irradiated inside of a main chamber.

The gases such as hydrogen, oxygen, carbon dioxide are supplied from the raw material supply unit 100 and passes through the inside of the main chamber, the quantum energy generated by the radiation moderation phenomenon is irradiated to the gases. By quantum energy irradiation, the covalent bonds of the gases material are dissociated, and the gases material may become atoms, cations or anions.

A second ion generator 200B includes the second quantum energy generator 230 of the braking radiation type,
A third quantum energy generator 270 of high-voltage discharge method includes a main body (a housing) 271, a first saddle gradient coil 272-1a having first discharge electrodes 272-2a, a second gradient saddle coil 272-1b having second discharge electrodes 272-2b, a modified Trigger coil 273 having a ground electrode 273a, a modified Trigger coil 274 having third discharge electrodes 274a, a high-voltage generator 278 and a conducting wire 279.

After insulating the inner surface of the main body 271 with an insulator, the first gradient coil 272-1a having the first discharge electrode 272-2a and the second gradient saddle coil 272-1b having the second discharge electrode 272-2b are installed in surface contact with the inner surface of the main body, and are installed to face each other at upper and lower intervals, and the wound coil directions are opposite to each other.

The modified Trigger coil 273 having the ground electrode 273a is installed at intervals from Gradient saddle coil 272-1a, 272-1b in the inner center direction.

The modified Trigger coil 274 having the third discharge electrode 274a is installed at intervals from the modified Trigger coil 273 in the inner center direction.

In the high-voltage generator 278 installed on the outside side, when the generated pulsed high-voltage is supplied to the first gradient saddle coil 272-1a having the first discharge electrodes 272-2a, the second gradient saddle coil 272-1b having the second discharge electrodes 272-2b, the modified Trigger coil 273 having the ground electrodes 273a, and the modified Trigger coil 274 having the third discharge electrodes 274a, between the coils, the electromagnetic field of the pulse form of the opposite direction is generated, and the electromagnetic fields of the pulse form of the opposite direction at the center distance are overlapped and offset (dissipated), and pulsating orient energy is generated in the state of the zero magnetic field, and it is irradiated inside the main body 271, and at the same time a high-voltage discharge is initiated between the coils 272,273,274.

The gases such as hydrogen, oxygen, carbon dioxide are supplied from the raw material supply unit 100 and passes through the inside of the main chamber, the quantum energy and the high-voltage discharge energy are irradiated to the gases.

By quantum energy irradiation and the high-voltage discharge energy, the covalent bonds of the gases material are dissociated, and the gases material may become atoms, cations or anions.

A third ion generator 200C includes the third quantum energy generator 270 of the high-voltage discharge method.

An ion generator 200 includes the first ion generator 200A, the second ion generator 200B and the third ion generator 200C.

A first dissolving device 310 includes a first circulating pump 311, a flow control valve 312, a venturi ejector 313, a pressure detection sensor 314, a main body (chamber) 315, a first defoamer 318 and a circulation tube 319.

The first defoamer 318 includes a first quantum energy generator consisting of a first quantum energy generating coil 316a, a second quantum energy generating coil 316b and a pulsed power supply unit 317.

By operating the first circulation pump 311, the solution stored in a main body 501 of a quantum energy transfer device 500A is sucked and pressurized, and hydrogen ions (H+, H−), oxygen ions (O+, O−) or nitrogen ions (N+, N−, NO) generated in the ion generator 200 are supplied in the process of passing through the venturi ejector 313.

Air bubbles mixed in the solution are primarily destroyed and decompressed during the process of passing through a 1 mm pore disperser 315a installed in the inner center of the main body 315 by supplying hydrogen ions (H⁺, H⁻), oxygen ions (O⁺, O⁻) or nitrogen ions (N⁺, N⁻, NO) into the main body 315.

In the pulse-type power supply unit 317 installed on the outside side, the generated pulsed high-voltage is supplied to the first and second quantum energy generating coils 316a, 316b of Uniform saddle coil.

The first and second quantum energy generating coils 316a, 316b are installed to face each other at interval, and the wound coil directions are opposite to each other.

Between the coils, the electromagnetic field of the pulse form of the opposite direction is generated, and the electromagnetic fields of the pulse form of the opposite direction at the center distance are overlapped and offset (dissipated), and a quantum energy is generated in the state of the zero magnetic field, and it is irradiated inside the main body 315.

The quantum energy irradiates to the solution inside the body 315 to depocalyse (destroy) the air bubbles contained in the solution, as the result, the hydrogen ions (H⁺, H⁻), oxygen ions (O⁺, O⁻) or nitrogen ions (N⁺, N⁻, NO) may be primary dissolved.

A second dissolving device 320 includes a second circulating pump 321, a flow control valve 322, a pressure detection sensor 323, a chamber 324, a second defoamer 327 and a circulation tube 324a.

The second defoamer 327 includes a second quantum energy generator consisting of a first quantum energy generating coil 325a, a second quantum energy generating coil 325b and a pulsed power supply unit 326.

When a control panel 600 supplies power to the second circulation pump 321, the second circulation pump 321 is operated so that the process fluid in which hydrogen ions (H⁺, H⁻), oxygen ions (O⁺, O⁻) or nitrogen ions (N⁺, N⁻, NO) are primarily dissolved in the first dissolving device 310 is sucked and pressurized by the second circulation pump 321 and introduced into the second defoamer 327 to which the second quantum energy is irradiated through the flow rate adjusting valve 322.

The chamber 324 of the second dissolving device 320 to which the second quantum energy is irradiated has a cylindrical shape having a predetermined diameter and a predetermined length, and is insulated from the inner surface of the cylindrical chamber 324 so that a first quantum energy generation coil 325a having Solenoid coil shape is installed to be in surface contact with the inner surface. A second quantum energy generation coil 325b having Trigger coil shape is installed at intervals, and a third quantum energy generation coil 325c having a modified RF coil shape is installed downward at intervals from the first and second quantum energy generation coils 325a and 325b.

Trigger voltage generator 326b is installed at one side of the outside of the main body, is connected to the pulsed power supply unit 326 with a conducting wire.

The first and second quantum energy generating coils 325a and 325b are connected to a pulsed power supply unit 326a through a conducting wire, and the third quantum energy generating coil 325c is connected to the Trigger voltage generator 326b through the conducting wire. The pulsed power supply unit 326a and the Trigger voltage generator 326b are also connected through a conducting wire.

The third quantum energy generation coil 325c starts discharging while pulse energy is transferred between the first and second quantum energy generation coils 325a and 325b having polarities different from those of the magnetic field. The third quantum energy generation coil 325c starts main discharge at a lower voltage than the high voltage pulse applied to the first quantum energy generation coil 325a and the second quantum energy generation coil 325b.

At the same time, when a high voltage pulsed power supply unit 326a generates a pulse-type high voltage to the first quantum energy generation coil 325a and the second quantum energy generation coil 325b installed such that winding directions of the coils are opposite to each other through a conducting wire, a pulse-type magnetic field in opposite directions is generated in the first quantum energy generation coil 325a and the second quantum energy generation coil 325b at an angle of 90° with respect to a flow direction of a current, pulse-type magnetic fields in opposite directions are overlapped and offset (dissipated) at a center distance of the first quantum energy generation coil 325a and the second quantum energy generation coil 325b, and quantum energy is generated in a zero magnetic field state and irradiated to the introduced fluid. As a result, a shock wave is generated by the discharge, and at the same time, bubbles containing hydrogen ions (H⁺, H⁻), oxygen ions (O⁺, O⁻) or nitrogen ions (N⁺, N⁻, NO), which are not destroyed in the first dissolving device 310 passing through between the first quantum energy generation coil 325a, the second quantum energy generation coil 325b, and the third quantum energy generation coil 325c, are destroyed by the discharge shock wave, and the water hammer pressure generated by the destruction of the bubbles efficiently further destroys the bubbles adjacent to the discharge shock wave.

A first ion separator 400A includes a positive charge collector 410 and a first quantum energy generaton device 420. The positive charge collector 410 includes a main body 401, a first power supply unit 411, a coil 412 and multiple positive charge collecting electrodes 410. The main body 401 of the first ion separator 400A may have a cylindrical shape with conical upper and lower parts, or a rectangular parallelepiped shape with rectangular pyramid shapes at upper and lower parts.

A plurality of first positive charge collecting electrodes 413 supplied with power generated by a power supply unit 411 of the positive charge collector 410 are installed in upper and lower holders 401a and 401b at intervals from each other inside the main body 401 and at intervals from the upper, lower, left, and right inner surfaces of the main body 401.

The positive charge collector 410 includes the power supply unit 411, the first coil 412, the positive charge collecting electrodes 413, and a conducting wire 414.

In addition, a first Cuffs coil 421 of the first quantum energy generator 420 is installed in outer upper area of the main body 401, a second Cuffs coil 422 is installed at in outer lower area of the main body 401, and a power supply unit 423 is installed at an interval to supply the power generated from the power supply unit 423 to the first Cuffs coil 421 and the second Cuffs coil 422 through a conducting wire 424.

Pulse-type variable power is supplied to the first Cuffs coil 421 and the second Cuffs coil 422 wound in opposite directions through a wire from the power supply unit 423 of the first quantum energy generator 420. In the first Cuffs coil 421, a pulsed electromagnetic field (PEMF) is irradiated in the lower direction of the main body 401 at an angle of 90 degrees with the current flow direction, and in the second Cuffs coil 422, a pulsed electromagnetic field (PEMF) is irradiated in the upper direction of the main body 401 at an angle of 90 degrees with the current flow direction.

As a result, the pulse-type magnetic fields irradiated in opposite directions at the central distance between the first Cuffs coil 421 and the second Cuffs coil 422 overlap and offset (dissipate), and the pulsation quantum energy generated in the zero magnetic field state is irradiated into the central distance space between the first Cuffs coil 421 and the second Cuffs coil 422 inside the main body 401.

In the positive charge collector 410, the output positive (+) terminal of the power supply unit 411 is directly connected to the first coil 412 through a conducting wire, but the output negative (−) terminal of the power supply unit 411 is connected to the plurality of first positive charge collecting electrodes 413a, 413c, and 413e through a conducting wire, and each of second positive charge collecting electrodes 413b, 413d, and 413f is separated from the first positive charge collecting electrodes 413a, 413c, and 413e at an interval so that current does not flow.

The first coil 412 is directly supplied with positive (+) power of the power supply unit 411, and negative (−) power is not supplied to the first coil 412 installed to be connected to the second positive charge collecting electrodes 413b, 413d, and 413f installed separately from the first positive charge collecting electrodes 413a, 413c, and 413f connected to the output negative (−) terminal of the power supply unit 411. Therefore, between the first positive charge collecting electrodes 413a, 413c, and 413e and the second positive charge collecting electrodes 413b, 413d, and 413f, there is a strong attraction to supply negative (−) power.

As a result, hydrogen ions (H⁺, H⁻), oxygen ions (O⁺, O⁻) or nitrogen ions (N⁺, N⁻, NO) generated by dissociating hydrogen molecules, oxygen molecules or nitrogen molecules in the ion generators 200 are introduced into the first ion separator 400A and pass through between the first and second collection electrodes, and hydrogen ions (H⁺), oxygen ions (O⁺) or nitrogen ions (N⁺) are adsorbed (captured) to the first charge collecting electrodes 413a, 413c, and 413e through which negative (−) currents flow and the second charge collecting electrodes 413b, 413d, and 413f in which attraction acts.

On the contrary, hydride ions (H⁻), oxygen ions (O⁻) or nitrogen ions (N⁻) may be supplied to the energy transfer device 500 by the pressing force of the second circulation pump 321 by acting a repulsive force with the first charge collecting electrodes 413a, 413c, and 413e through which negative (−) current flows.

The second ion separator 400B includes a negative charge collector 430 and a second quantum energy generaton device 440. The negative charge collector 430 includes a main body 402, a first power supply unit 431, a coil 432 and multiple negative charge collecting electrodes 433. And the second quantum energy generaton device 440 includes a first Cuffs coil 441, a second Cuffs coil 442 and a power supply unit 443.

The second ion separator 400B includes a negative charge collector 430 and a second quantum energy generaton device 440. The negative charge collector 430 includes a main body 402, a first power supply unit 431, a coil 432 and multiple negative charge collecting electrodes 433. And the second quantum energy generaton device 440 includes a first Cuffs coil 441, a second Cuffs coil 442 and a power supply unit 443.

The main body 402 of the second ion separator 400B may have a cylindrical shape with conical upper and lower parts, or a rectangular parallelepiped shape with rectangular pyramid shapes at upper and lower parts.

The plurality of first negative charge collecting electrodes 433 supplied with power generated by the power supply unit 431 of the negative charge collector 430 are installed in the upper and lower holders at intervals from each other inside the main body 402 and at intervals from the upper, lower, left, and right inner surfaces of the main body 402.

In addition, the first Cuffs coil 441 of the second quantum energy generator 440 is installed in the outer upper area of the main body 402, the second Cuffs coil 442 is installed in the outer lower area of the main body 402, and the power supply unit 443 is installed at an interval to supply the power generated from the power supply unit 443 to the first Cuffs coil 441 and the second Cuffs coil 442 through the conducting wire.

Pulse-type variable power is supplied to the first Cuffs coil 441 and the second Cuffs coil 442 wound in opposite directions through a wire from the power supply unit 443 of the second quantum energy generator 440. In the first Cuffs coil 441, a pulsed electromagnetic field (PEMF) is irradiated in the lower direction of the main body 402 at an angle of 90 degrees with the current flow direction, and in the second Cuffs coil 442, a pulsed electromagnetic field (PEMF) is irradiated in the upper direction of the main body 402 at an angle of 90 degrees with the current flow direction.

As a result, the pulse-type magnetic fields irradiated in opposite directions at the central distance between the first Cuffs coil 441 and the second Cuffs coil 442 overlap and offset (dissipate), and the pulsation quantum energy generated in the zero magnetic field state is irradiated into the central distance space between the first Cuffs coil 441 and the second Cuffs coil 442 inside the main body 402.

In the negative charge collector 430, the output negative (−) terminal of the power supply unit 431 is directly connected to the second coil 432 through a conducting wire, but the output positive (+) terminal of the power supply unit 431 is connected to a plurality of first charge collecting electrodes 433a, 433c, and 433e through a conducting wire, and each of second charge collecting electrodes 433b, 433d, and 433f is separated from the first charge collecting electrodes 433a, 433c, and 433e at an interval so that current does not flow.

The second coil 432 is directly supplied with negative (−) power of the power supply unit 431, and positive (+) power is not supplied to the second coil 432 installed to be connected to the second charge collecting electrodes 433b, 433d, and 433f installed separately from the first charge collecting electrodes 433a, 433c, and 433f connected to the output positive (+) terminal of the power supply unit 431. Therefore, between the first charge collecting electrodes 433a, 433c, and 433e and the second charge collecting electrodes 433b, 433d, and 433f, there is a strong attraction to supply positive (+) power.

As a result, hydrogen ions (H⁺, H⁻), oxygen ions (O⁺, O⁻) or nitrogen ions (N⁺, N⁻, NO) generated by dissociating hydrogen molecules, oxygen molecules or nitrogen molecules in the first, second, and third ion generators 200A, 200B, and 200C are introduced into the first and second ion separators 400A and 400B and pass through between the first and second collection electrodes, and hydrogen ions (H⁻), oxygen ions (O⁻) or nitrogen ions (N⁻) are adsorbed (captured) to the first charge collecting electrodes 433a, 433c, and 433e through which positive (+) currents flow and the second charge collecting electrodes 433b, 433d, and 433f in which attraction acts.

A third ion separator 400C includes a reactor 440, a condenser 450 and a positive charge collector 457. The reactor 440 includes a main body 403, a jacket 403a, a cooling water circulation line 444a, a hot water circulation line 444b, a municipal water or purified water supply line 445, a raw material input line 446, and a stirrer 447. The lower part of the main body 403 of the reactor 440 is conical, the upper part of the main body 403 is inclined, and the main body 403 may have a dual-structure cylindrical shape including a jacket 403a. The cooling water supply and exhaust line 444a, and the hot water supply and exhaust line 444b may be connected and installed on one side of the side lower portion and one side of the side upper portion of the jacket 403a.

The condenser 450 includes a main body 451, a cooling water circulation line 451a, and a exhaust line 452 of hydrogen ions (H⁻) or oxygen ions (O⁻).

The positive charge collector 457 includes a plurality of first cathodes (453a, 453b, 453c, 453d), a first coil (454; the load), a second power supply unit 455, and conducting wires 456.

The electronic valve 445a of the municipal water or the purified water supply line 445 is opened and the municipal water or the purified water is filled with 60 to 75% of the volume of the main body 403 of the container 440. The electronic valve of the hot water supply and exhaust line 444a is opened, and the hot water are circulated from the lower supply line of the jacket 403a to the top exhaust line 444a and heat the municipal water or the purified water filled with appropriate amount inside the main body 403 to 40 to 60 degrees Celsius. The mixer 447 is operated, and the mixer 447 stirs the municipal water or the purified water in the range of 30 to 60 RPM. The pulse-type DC power generated by the power supply unit 443 is supplied to the first electrode 441 and the second electrode 442 installed on the inner side surface of the reactor 440.

The cover of the hopper 446 is opened, and the metal hydrate in which one kind material is selected from the lithium hydride (LiH), the sodium hydride (NaH) and the potassium hydride (KH) is supplied to the gravity difference to the main body 403. Hydride ions (H⁻) may be generated by a hydrolysis reaction and an electrolysis reaction between the metal hydrate and the water. As the pulse-type DC power generated by the first power supply unit 443 is supplied to the first electrode 441 and the second electrode 442, the metal hydrate introduced into the main body 403 is subjected to an electrolysis reaction to generate a hydrogen ions (H⁻).

Hydrogen ions (H⁻) generated by the hydration reaction flow into the main body 451 of the condenser 450 through the supply line 452 and are cooled by indirect heat exchange with cooling water introduced into the cooling water supply line and the exhaust line 451 installed on one side of the lower and upper sides of the body 451 to condense and liquefy the water vapor of water molecules mixed with hydrogen ions (H⁻).

The liquefied water is discharged to the outside through a condensate exhaust line installed under the left cell 451a of the main body 451 so that hydrogen ions (H⁻) and water vapor are separated and introduced into the quantum energy transfer devices. In this process, positive charges may be collected on the first and second cathodes to suppress a reaction between hydride ions (H⁻) and a small amount of lithium ions (Li⁺) or sodium ions (Na⁺) and potassium ions (K⁺).

Specifically, negative (−) terminals on the output side of the power supply unit 455 of the positive charge collector 457 are connected to each of the plurality of first negative electrodes 453a-1, 453b-1, 453c-1, and 453d-1 of the positive charge collector, positive (+) terminals are connected to the first coil 454, and another electric wire of the first coil 454 is connected to the second negative electrodes 453a-2, 453b-2, 453c-2, and 453c-2. The second negative electrodes 453a-2, 453b-2, 453c-2, and 453c-2 are installed to be separated from the first negative electrodes 453a-1, 453b-1, 453c-1, and 453d-1. Negative (−) power is supplied to the first negative electrodes 453a-1, 453b-1, 453c-1, and 453d-1, and no power is supplied to the second negative electrodes 453a-2, 453b-2, 453c-2, and 453d-2, therefore, between the first negative electrodes 453a-1, 453b-1, 453c-1, and 453d-1 and the second negative electrodes 453a-2, 453b-2, 453c-2, and 453d-2, there is a strong attraction to supply negative (−) power.

Therefore, lithium ions (Li⁺), sodium ions (Na⁺), and potassium ions (K⁺) are collected in the first and second cathodes inside the condenser, and hydrogen ions (H⁻) are separated from the cations by repulsive force and introduced into the quantum energy transfer devices by the pressing force of the air FAN.

A first quantum energy transfer device 500A includes an ion gas supply line 501a, a municipal water or purified water supply line 501b, a solution supply line 501c in which ions are dissolved, a circulation and exhaust line 505a and 505b, a first main body 501, a second main body 502, a stirrer 503, a circulation pump 504, a first quantum energy generation device 510, and a second quantum energy generation device 520. The first quantum energy generation device 510 includes a first quantum energy generation coil 511, a second quantum energy generation coil 512, a first power supply unit 513 for supplying power in a pulse form, and a conducting wire 514. The second quantum energy generation device 520 includes a third quantum energy generation coil 521, a fourth quantum energy generation coil 522, a second power supply unit 523 for supplying power in a pulse form, and a conducting wire 524.

A solution containing hydrogen ions (H⁺, H⁻) or oxygen ions (O⁺, O⁻) in the dissolving device 300 is sent to the supply line 501c by the pressing force of the pressure pump 321, followed by 70-80% of the internal volume of the main body 501 through the venturi ejector 506.

Alternatively, a first solution in which hydrogen ions (H⁻) or oxygen ions (O⁻) supplied from the first and third ion separators (400A, 400C) or hydrogen ions (H⁺) or oxygen ions (O⁺) supplied from the secondary ion separator 400B are mixed with purified water (municipal water) may be filled to 70 to 80% of the internal volume of the main body 501 through the supply line 501B and the venturi ejector 506.

Alternatively, a second solution in which hydrogen ions (H⁻) or oxygen ions (O⁻) supplied from the first and third ion separators (400A, 400C) or hydrogen ions (H⁻) or oxygen ions (O—) supplied from the secondary ion separator 400B are mixed with purified water (municipal water) may be filled to 70 to 80% of the internal volume of the main body 501 through the supply line 501B and the venturi ejector 506.

In this process, the level sensor detects the water level and transmits it to the control panel 600, and the control panel 600 stops the pressure pump 321 and supplies power to the stirrer 501 to rotate the first solution or the second solution supplied to the main body 501.

At the same time, when pulsed direct current power is supplied from the first power supply 513 to the first and second quantum energy generating coils 511 and 512 wound in opposite directions through a wire, an electromagnetic field in the form of a pulse is generated at an angle of 90 degrees in the current flow direction and transferred to the first solution or the first solution.

As a result, electromagnetic fields in the form of pulses in opposite directions overlap and offset (dissipate) in the center of the first body 501, generating quantum energy in a zero magnetic field state, and the quantum energy above can be irradiated to the solution.

Subsequently, when power is supplied to the circulation pump 504 from the control panel 600, the solution irradiated with electromagnetic fields and quantum energy is sucked and pressurized inside the first body 501 and supplied to the second body 502.

The third quantum energy generating coils 521 and the fourth quantum energy generating coils 522 may be wound in opposite directions, and an inlet and an outlet hole may be perforated between the coil and the coil wound with a predetermined winding number.

The pulse-type power generated by the second power supply unit 513 is applied to the third quantum energy generating coils 521, in which the coils are wound in opposite directions, and the pulse-type electromagnetic field generated at an angle of 90 degrees in the current flow direction is installed to face each other. In addition, the opposite pulse-type electromagnetic energy generated between the third quantum energy generating coils 521 and the fourth quantum energy generating coils 522 may be irradiated to the solution passing through the inlet and outlet holes. The electromagnetic energy in the form of pulses in opposite directions overlap and offset (dissipate), and the pulse-type quantum energy generated in the zero magnetic field state is irradiated and continues to circulate for a certain period of time, before the electric valve 505a installed on the circulation line 505 above the second body 502 is closed and the electric valve 505b is opened to supply the solution to which the electromagnetic field and quantum energy are irradiated.

A second quantum energy transfer device 500B includes a main body 503, an ion gas supply line 504, a first quantum energy generation device 530, an upper first rotating body motor 544, a pneumatic cylinder 544a, a first rotating disk 544b, a lower second rotating body motor 545, a second rotating disk 545b, and a second quantum energy generation device 540. The first quantum energy generation device 530 includes a first quantum energy generation coil 531, a second quantum energy generation coil 532, a first power supply unit 533, and a conducting wire 534. The second quantum energy generation device 540 includes a third quantum energy generation coil 541, a fourth quantum energy generation coil 542, a pulse-type power supply unit 543, and a conducting wire.

When the control panel 600 supplies power to the motor 545a for the second rotating body 545, the motor 544a for the first rotating body 544, and the pneumatic or hydraulic cylinder 546, the motor 545a of the second rotating body 545 is operated to rotate the motor shaft and the second rotating disk 545c.

At the same time, the pneumatic or hydraulic cylinder 546 installed at the center of the upper portion of the main body 503 is operated to lower the first rotating body 544 composed of the motor 544a, the shaft 544b, and the turntable 544c to the close distance (10 to 20 cm) of the quantum energy treatment object loaded on the turntable 545c of the second rotating body 545 inside the main body 503,
and then the motor 544a of the first rotating body 544 is operated in the control panel to rotate the shaft 544b connected to the motor shaft and the turntable 544c to rotate the hydrogen ion (H⁻) or oxygen ions (O⁻) supplied from the first ion separator 400A, or oxygen ions (O⁺), the hydrogen ions (H⁺), the nitrogen ions (N⁺) or the nitrogen oxide ions (NO) supplied from the second ion separator 400B in a state where the ions are supplied or not supplied to one side of the upper portion of the main body 503 of the second quantum energy transfer device 500B by the pressing force of the air FAN 157.

At the same time, the pulse-shaped electromagnetic fields generated by the first power supply unit 533 are irradiated from the upper portion to the lower portion and from the lower portion to the upper portion to the loaded quantum energy transfer target in the first quantum energy generation coil 531 of the plurality of first quantum energy generators installed to be in surface contact with the rear surface of the turntable 544c of the first rotating body 544 and the second quantum energy generation coil 532 installed to be in surface contact with the rear surface of the turntable 545c of the second rotating body 545 so that the pulse-shaped electromagnetic fields generated by the first power supply unit 533 are overlapped and offset (dissipated) in the center direction of the loaded target and transferred to the loaded object.

A third quantum energy transfer device 500C includes a main body 504, a screw driving motor 505, a screw 506, a powder inlet 507, a powder outlet 507a, a supply fan 508, an ion gas supply line 509 and a third quantum energy generation device 550C. The third quantum energy generation device 550C includes a plurality of first quantum energy generation coils 551 (551a˜551j), a plurality of second quantum energy generation coils 552 (552a˜552j), a first power supply unit 553, and a conducting wire 554.

Hydrogen ions (H⁻), oxygen ions (O⁻), nitrogen ions (N⁻), nitrogen oxide ions (NO), hydrogen ions (H⁺), oxygen ions (O+) or nitrogen ions (N⁺) supplied from the ion separators (400A, 400B, 400C), may be supplied to the venturi neck part (powder outlet) 507a connected to the supply FAN 508 of the third quantum energy transfer device 500C by the suction power of the supply FAN 509 or by the pressing force of the air FAN 137.

At the same time, when the quantum processing object (powder) is introduced into the main body 504 by the blade of the supply FAN 508, and discharged to the outlet 507a by the screw 506 driven by the drive motor 505′, the pulse-type power generated by the pulse-type power supply unit 553 is supplied through the wire to the multiple first quantum energy generating coils 551 and second quantum energy generating coils 552 installed to face each other in the circumferential direction of the outer circumferential surface of the main body 504, an electromagnetic field in the form of a pulse generated at a 90 degree angle in the current flow direction is irradiated from the top to the bottom of the main body 504 in the first quantum energy generating coil 551, and the second quantum energy generating coil 552 is irradiated from the bottom of the main body 504 to the top of the main body 504, and the pulse-type electromagnetic fields in opposite directions overlap and offset (dissipate) in the screw shaft portion to generate quantum energy in a zero magnetic field state.

When oxygen ions (O⁺, O⁻) are supplied from the ion gas supply line 509 while the generated quantum energy is irradiated to the quantum energy treatment object, the powder raw material may be mixed with oxygen ions (O⁺, O⁻) by screw driving inside the main body 504 to be perform an oxidation reaction.

When hydride ions (H⁻) or hydrogen ions (H⁺) are supplied from the ion gas supply line 509, the powder raw material may be mixed with hydride ions (H⁻) or hydrogen ions (H⁺) by screw driving inside the main body 504 to be perform a reduction reaction.

When nitrogen ions (N⁺, N⁻, NO) is supplied from the ion gas supply line 509, the powder raw material may be mixed with the nitrogen ions (N⁺, N⁻, NO) by screw driving inside the main body 504 to be perform a nitrification reaction.

FIG. 2 is a cross-sectional view illustrating the raw material supply unit shown in FIG. 1.

Referring to the accompanying drawings, the raw material supply unit 100 includes: a first raw material supply unit 110 for supplying air; a second raw material supply unit 120 for adjusting the pressure and flow rate of oxygen filled at a high pressure and supplying the oxygen; a third raw material supply unit 130 including a 3-1 raw material supply unit 130a for injecting a hydrogen generating raw material into the municipal water or the purified water and generating and supplying hydrogen by stirring while irradiating quantum energy, and a 3-2 raw material supply unit 130b for injecting an oxygen generating raw material and a catalyst material into the municipal water or the purified water and generating and supplying oxygen by stirring while irradiating quantum energy; a fourth raw material supply unit 140 for adjusting the pressure and flow rate of hydrogen filled at a high pressure and supplying the hydrogen; and an inert gas supply unit 150 for adjusting the pressure and flow rate of nitrogen, carbonic acid and argon gas.

A first raw material supply unit 110 includes a filter housing 111, a supply fan 112, a flow regulator 113, a valve 114, and an air supply line 115, and the supply fan 112 is operated to suck and pressurize in external air. In the process, the HEPA filter 111a mounted inside the filter housing 111 filters fine dust and adjusts the air supply flow rate from the flow regulator 113 to the appropriate flow rate and supplies through the supply line 115 to the ion generator 200.

A second raw material supply unit 120 includes a pressure regulator 122, a flow regulator 123, an electronic valve 124, and an air supply line 125, and oxygen gas is supplied to the pressure regulator 122 by opening the main valve (not shown) of the container filled at high pressure (120 kg/cm²), depressurizing to the range of 50 mm Aq˜350 mm Aq in the pressure regulator 122, and supplying to the flow regulator 123, and after adjusting the flow rate at an appropriate flow rate in the flow regulator 123, supplied to the supply line 125 through the electronic valve 124 and supplied to the ion generator 200.

A fourth raw material supply unit 140 includes a container 141 filled with hydrogen gas, a pressure regulator 142, a flow regulator 143 and an electronic valve 144.

Hydrogen gas (H₂) is filled in container 141 with high pressure (120 kg/cm²), the main valve (not shown) of the container 141 is opened and supplied to the pressure regulator 142, and supplied to the flow regulator 143 after depressurization in the range of 50 mm Aq˜350 mmAq, and after adjusting the flow rate to an appropriate flow rate in the flow regulator 143, it is supplied to the mixer 154 through the supply line and mixed with the inert gas and then supplied to the ion generator 200.

A first inert gas supply unit 151 of the inert gas supply unit 150 includes a container 151a filled with nitrogen gas, a pressure regulator 151b, a flow regulator 151c and an electronic valve 151d. Nitrogen gas (N₂) is supplied to the pressure regulator 151b after opening the main valve (not shown) of the container 151a filled with high pressure (120 kg/cm²), and after depressurizing the pressure regulator 151b to the range of 50 mm Aq˜350 mmAq, supplying it to the flow regulator 151 c, and after adjusting the flow rate to the appropriate flow rate in the flow regulator 151c, it is supplied to the mixer 154 through the supply line.

A second inert gas supply unit 152 of the inert gas supply unit 150 includes a container 152a filled with carbon dioxide gas, a pressure regulator 152b, a flow regulator 152c and an electronic valve 152d.

Carbon dioxide gas (CO₂) is supplied to the pressure regulator 152b after opening the main valve of the container 152a filled with high pressure (120 kg/cm²), and after depressurizing the pressure regulator 152b to the range of 50 mm Aq˜350 mmAq, supplying it to the flow regulator 152c, and after adjusting the flow rate to the appropriate flow rate in the flow regulator 152c, it is supplied to the mixer 154 through the supply line.

A third inert gas supply unit 153 of the inert gas supply unit 150 includes a container 153a filled with argon gas, a pressure regulator 153b, a flow regulator 153c and an electronic valve 153d. Argon gas (Ar) is supplied to the pressure regulator 153b after opening the main valve of the container 153a filled with high pressure (120 kg/cm²), and after depressurizing the pressure regulator 153b to the range of 50 mm Aq˜350 mmAq, supplying it to the flow regulator 153c, and after adjusting the flow rate to the appropriate flow rate in the flow regulator 153c, it is supplied to the mixer 154 through the supply line.

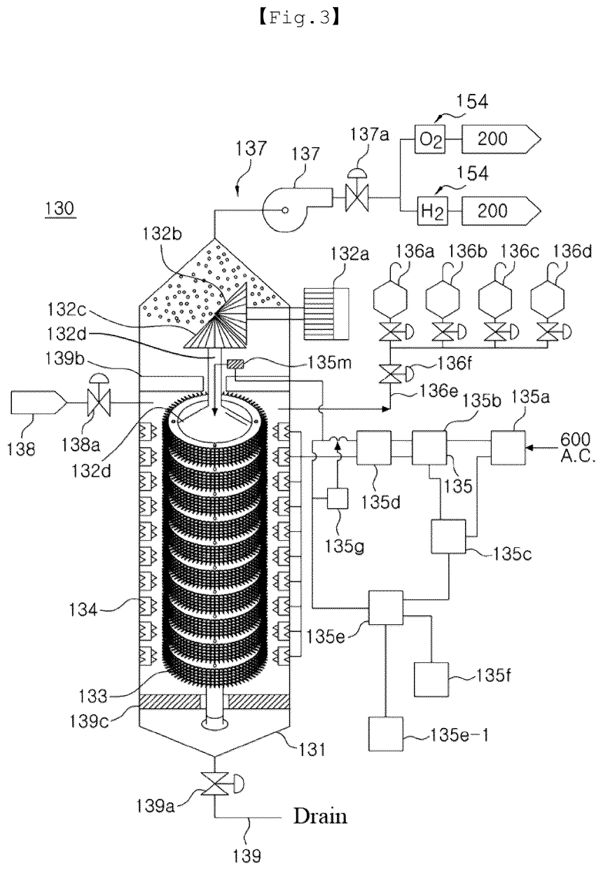

FIG. 3 is a schematic diagram illustrating a third raw material supply unit 130 unit of the raw material supply unit 100 of FIG. 1. Referring to the accompanying drawings, the third raw material supply unit 130 includes a 3-1 raw material supply unit 130a and a 3-2 raw material supply unit 130b. The 3-1 raw material supply unit 130a includes a main body 131, a stirrer 132, a motor 132a, a first bevel gear 132b, a second bevel gear 132c, a rotating shaft 132d, a first quantum energy generation coil 133, a second quantum energy generation coil 134, a power supply unit 135, storage tanks 136a and 136b, an exhaust fan 137, a municipal water or purified water supply line 138 and an exhaust line 139, and the storage tanks 136a and 136b store hydrogen generating raw materials such as borohydride salt (MBH₄) and triborohydride salt (MB₃H₈).

The first quantum energy generating coil 133 is installed on the outer circumferential surface of the rotating shaft 132d of the stirrer, and the second quantum energy generating coil 134 is installed on the inner circumferential surface of the main body 131.

While stirring the municipal water or purified water stored in the main body 131 by operating the stirrer 132, pulse-shaped power generated by the power supply unit 135 is supplied to the first and the second quantum energy generation coil 134. As a result, the pulse-shaped electromagnetic field and the electromagnetic field in the opposite direction overlap and offset (dissipate) to produce pulsating quantum energy generated in a zero magnetic field state.

While the pulsating quantum energy is irradiating municipal water or purified water, the hydrogen generating raw materials stored in the storage tanks 136a, 136b are supplied, and the hydrogen generated by the hydrolysis reaction with municipal water or purified water is supplied to a mixer 154 using the exhaust fan 137.

The 3-2 raw material supply unit 130b includes a main body 131, a stirrer 132, a motor 132a, a first bevel gear 132b, a second bevel gear 132c, a rotating shaft 132d, a first quantum energy generation coil 133, a second quantum energy generation coil 134, a power supply unit 135, storage tanks 136c and 136d, an exhaust fan 137, a municipal water or purified water supply line 138 and an exhaust line 139.

The storage tank 136c is stored oxygen generating raw materials such as sodium peroxide and potassium peroxide, and the storage tank 136d is stored catalyst materials such as ferric formate, ferric citrate and ferric ammonium citrate.

The first quantum energy generating coil 133 is installed on the outer circumferential surface of the rotating shaft 132d of the stirrer, and the second quantum energy generating coil 134 is installed on the inner circumferential surface of the main body 131.

While stirring the municipal water or purified water stored in the main body 131 by operating the stirrer 132, pulse-shaped power generated by the power supply unit 135 is supplied to the first and the second quantum energy generation coil 134. As a result, the pulse-shaped electromagnetic field and the electromagnetic field in the opposite direction overlap and offset (dissipate) to produce pulsating quantum energy generated in a zero magnetic field state.

While the pulsating quantum energy is irradiating municipal water or purified water, the oxygen generating raw materials and catalyst materials stored in the storage tanks 136c, 136d are supplied, and the oxygen generated by the hydrolysis reaction with municipal water or purified water is supplied to an ion generator 200 using the exhaust fan 137.

The shape of the main body 131 is a cylindrical shape or a rectangular pillar shape in which the upper and lower parts are inclined.

The motor 132a of the stirrer 132 is mounted on one side of the upper right side of the main body 131, and the rotation shaft of the motor is installed to extend to the center through the main body 131, and the first bevel gear 132b is installed at the end of the rotation shaft.

The gear teeth of the first bevel gear 132b are engaged with the second bevel gear 132c installed at the upper center of the rotating shaft 132d on which the first quantum energy generating coil 133 is installed, in which the gear teeth are in contact with the first bevel gear 132b at an angle of 90 degrees.

A plurality of first quantum energy generation coils 133 each other leaves interval in the rotating shaft 132d external side of the cylindrical shape to the columnar direction and it is installed.

A plurality of second quantum energy generation coils 134 of the first quantum energy generator is installed in the inner circumferential surface of the main body 131.

The plurality of first quantum energy generation coil 133 and the plurality of second quantum energy generation coils 134 installed to face each other are wound in opposite directions and have outer surfaces protruding in a needle shape, and the outer surfaces of the first quantum energy generation coil 133 and the second quantum energy generation coil 134 are coated with an insulator.

The rotating shaft 132d is inserted and installed into a perforated portion (not shown) at the center of the plurality of fixing bars 139b and 139c installed at the upper and lower portions inside the main body.

The exhaust line 139 is installed in the lower center of the main body 131, a hydrogen supply line for transporting hydrogen gas generated in the main body 131 is installed in the upper center of the main body 131, and the exhaust fan 137 is installed in the hydrogen supply line.

Raw material storage tanks 136a, 136b for storing hydrogen generating raw materials are installed on one side of the upper part of the main body 131 at an interval from the motor 132a of the stirrer 132, a supply line 136e for supplying hydrogen generating raw materials is installed on the center of the lower part of the raw material storage tanks 136a, 136b, an electronic valve 136f is installed on the supply line 136e, and the supply line 136e is installed to penetrate one side of the upper part of the main body 131.

The municipal water or the purified water supply line 138 is installed in the main body 131 left side upper one side through the main body 131.

The power supply unit 135 of the first quantum energy generator is installed in the right side upper one side of the main body 131.

Any one of stainless steel (STS304, STS316L), glass fiber molding foam (FRP), steel (SS400), and hastelloy is selected and used as a material of the main body 131.

The piping used as the municipal water and purified water supply line 138, the exhaust line 139, and the hydrogen raw material supply line 136e is any one of a carbon steel pipe for piping (SPP), an electric arc welded carbon steel pipe for piping (SPW), a carbon steel pipe for pressure service (SPPS), an alloy steel pipe for piping (SPA), a stainless steel pipe for piping (STSXT), a galvanized steel pipe for water service (SPPW), a plastic lining steel pipe, a copper pipe, a polyvinyl chloride pipe, and a polyethylene pipe.

### Hydrogen Production

The raw material stored in the first hydrogen generation raw material storage tank 136a is selected any one of borohydride salt (MBH₄), triborohydride salt (MB₃H₈), decahydrodecaborate salt (M₂B₁₀H₁₀), tridecahydrodecaborate salt (MB₁₀H₁₃), dodecahydrodecaborate salt (M₂B₁₂H₁₂), octadecahydroicosaborate salt (M₂B20H₈) and decaborane (B₁₀H₁₄) generating hydrogen gas by contact with water or hydrolysis reaction. Wherein M is an alkali metal, an alkaline earth metal, and any one material selected from aluminum, sodium, lithium, and potassium.

The metal hydride raw material of the first metal composite electrode stored in the second hydrogen generation raw material storage tank 136b is selected any one of magnesium hydride (MgH₂), calcium hydride (CaH₂), lithium hydride (LiH), lithium borohydride (LiBH₄), sodium borohydride (NaBH₄), calcium borohydride (KBH₄), ammonium borohydride (NH₄BH₄), tetramethylammonium borohydride ((CH₃)₄NH₄BH₄), magnesium borohydride (Mg(BH₄)₂), calcium borohydride (Ca(BH₄)₂), sodium aluminum hydride (NaAlH₄), lithium aluminum hydride (LiAlH₄), potassium aluminum hydride (KA1H₄) and their mixture.

The metal hydride of the present invention acts as a reducing agent and reacts with water to produce hydrogen.

In the operation of the 3-1 raw material supply unit 130a, first, when power is supplied from the control panel 600 to the electronic valve 138a installed on the municipal water or purified water supply line 138, the electronic valve 138a is opened to fill the main body 131 with the municipal water or purified water to an appropriate level at which the first and second quantum energy generation coils 133 and 134 of the first quantum energy generator are submerged, and when the appropriate level is detected by a level sensor (not shown) and transmitted to the control panel 600, the electronic valve 138a is turned off (closed), and then the supply of the municipal water or purified water is stopped.

The raw material stored in the first hydrogen generation raw material storage tank 136a or the raw material stored in the second hydrogen generation raw material storage tank 136b is introduced into the main body 131 in which the municipal water or purified water is swirled through an electron emitting part having an open appropriate amount measured using a load cell (not shown), when power is supplied from the control panel 600 to the motor 132a of the stirrer 132 while the electronic valve 136f is opened and a suitable amount of the measured material is introduced into the main body 131 in which the municipal water or the purified water swirls, the motor 132a rotates, the first bevel gear 132b installed at the end of the motor shaft rotates, the second bevel gear 132c engaged with the bevel gear 132b at an angle of 90 degrees rotates, the rotating shaft 132d connected to the second bevel gear 132c and the rotating shaft 132d rotate, and the plurality of first quantum energy generating coils 133 installed at intervals from each other in the circumferential direction of the outer surface of the rotating body simultaneously rotate, so that the solution supplied with the hydrogen generating raw material to the municipal water or the purified water is stirred and mixed by the stirring function of the alternating rotating shaft 132d, and hydrolysis reaction with the municipal water or the purified water occurs to generate hydrogen gas.

In addition, The DC power in the form of pulses is supplied from the power supply unit 135 of the first quantum energy generator to the first quantum energy generating coil 133 installed on the outer circumferential surface of the rotating shaft 132d and the second quantum energy generating coil 134 installed on the inner circumferential surface of the main body 131, a plurality of first quantum energy generating coils 133 installed on the outer surface of the rotating shaft 132d are installed through a roller bearing 135m installed on one lower side of the second bevel gear 132c.

The first quantum energy generating coil 133 installed at the same height to face the installation position of the second quantum energy generating coil 134, the winding directions of the coils are wound in opposite directions in the first quantum energy generating coil 133 and the second quantum energy generating coil 134.

The electromagnetic field of the pulsed electromagnetic field (PEMF) is generated in opposite directions at an angle of 90 degrees from the first quantum energy generating coil 133 and the second quantum energy generating coil 134, and applied to the mixed solution.

And at the same time, the electromagnetic fields in opposite directions overlap and offset (dissipate) at a central distance between the first quantum energy generating coil 133 and the second quantum energy generating coil 134, and pulsed quantum energy in the form of a pulse is generated in the zero magnetic field state, and a hydrogen gas is generated in the main body by accelerating hydrolysis reaction with water from municipal water or purified water while irradiating to the mixture.

The reaction formulas for generating hydrogen gas through hydrolysis with the above-mentioned water or purified water are as in formula 1, formula 2, formula 3, formula 4, formula 5, formula 6 and formula 7.

1) Reaction formula between the sodium borohydride (NaBH₄) and the municipal water or purified water

NaBH₄+2H₂O→NaBO₂+4H₂ formula 1

2) Reaction formula between the decaborane (B₁₀H₁₄) and the municipal water or purified water

B₁₀H₁₄+15H₂O→5B₂O₃+22H₂ formula 2

3) Reaction formula between the sodium dodecahydrododeca borate (Na₂B₁₂H₁₂) and the Municipal water or purified water

Na₂B₁₂H₁₂+19H₂O→2NaBO₂+5B₂O₃+25H₂ formula 3

4) Reaction formula between the sodium triborohydride (NaB₃H₈) and the municipal water or purified water

NaB₃H₈+5H₂O→NaBO₂+B₂O₃+9H₂ formula 4

5) Reaction formula between the magnesium dodecahydrododeca borate (Mg₂B₁₂H₁₂) and the municipal water or purified water

Na₂B₁₂H₁₂+19H₂O→Mg(BO₂)₂+5B₂O₃+25H₂ formula 5

6) Reaction formula between the potassium triborohydride (KB₃H₈) and the municipal water or purified water

KB₃H₈+5H₂O→KBO₂+B₂O₃+9H₂ formula 6

7) Reaction formula between the diammonium decahydrodecaborate ((NH₄)₂B₁₀H₁₀) and the municipal water or purified water

(NH₄)₂B₁₀H₁₀+16H₂O→2(NH₄)₂BO₂+4B₂O₃+21H2 formula 7

In addition, when the first hydrogen-generating raw material and the second hydrogen-generating raw material are introduced into the main body 131, the first hydrogen-generating raw material and the second hydrogen-generating raw material are precipitated at the lower portion of the main body 131 because the first hydrogen-generating raw material and the second hydrogen-generating raw material are larger than the specific gravity of water, and thus the stirrer 132 is installed and stirred, so that the hydrogen-generating raw material and the second hydrogen-generating raw material are in sufficient contact with the municipal water, and the drain valve 139a installed on the exhaust line 139 is opened every predetermined time to small amounts periodically discharge the first hydrogen-generating raw material and the second hydrogen-generating raw material and the reaction by-product (NaBO₂, B₂O₃, Ca(OH)₂) precipitated at the bottom of the main body 131.

### Oxygen Production

The oxygen generating material stored in the storage tank 136c and the catalyst material stored in the storage tank 136d are mixed with municipal water or purified water to generate oxygen through a hydrolysis reaction, and any one of sodium peroxide, potassium peroxide, perchloric acid, calcium peroxide, calcium permanganate, potassium permanganate, magnesium peroxide, sodium percarbonate, and sodium perborate is selected and stored as the oxygen generating material stored in the storage tank 136c.

The catalyst material for generating oxygen stored in the catalyst material storage tank 136d includes any one of water-soluble iron compounds such as ferric formate, ferric citrate, ferric ammonium citrate, ferrous gluconate, ferric citrate, ferrous acetate, and ferrous chloride (FeCl₂), ferric chloride (FeCl₃), ferric oxalate, ferrous iodide, ferrous lacate, ferric nitrate, ferric glycophosphate, ferric orthophosphate, and ferrous sulfate.

In the operation of the 3-2 raw material (oxygen) supply unit 130b, first, when power is supplied from the control panel 600 to the electronic valve 138a installed on the municipal water supply line 138, the electronic valve 138a is opened to fill the main body 131 with the municipal water or purified water to an appropriate level at which the first and second quantum energy generation coils 133 and 134 of the first quantum energy generator are submerged,
and when the appropriate level is detected by a level sensor (not shown) and transmitted to the control panel 600, the electronic valve 138a is turned off (closed) to stop the supply of the municipal water or purified water.

Subsequently, any one of the oxygen generators, such as sodium peroxide, potassium peroxide, perchloric acid, calcium peroxide, calcium permanganate, magnesium peroxide, sodium percarbonate, sodium percarbonate, etc., stored in the storage tank 136c, and the appropriate amount measured in the load cell (not shown) is introduced into the body 131 where the water or purified water flows through the electronic valve 136f, and the stirrer 132 is operated to agitate the oxygen generator injected into the water or purified water while generating oxygen through the hydrolysis reaction.

At the same time, any one material selected from water-soluble iron compounds such as ferric formate, ferric citrate, ferric ammonium citrate, ferrous gluconate, ferric citrate, ferrous acetate, and ferrous chloride, ferric chloride, ferric oxalate, ferrous iodide, ferrous lacate, ferric nitrate, ferric glycophosphate, ferric orthophosphate, ferrous sulfate, and the like is weighed in a load cell (not shown) and then introduced into vortexing municipal water or purified water through an electronic valve to be hydrolyzed.

The reaction formulas for generating oxygen gas through hydrolysis with the above-mentioned water or purified water are as in formula 1, formula 2, formula 3-formula 4 and formula 5.

Hydrolysis reaction of sodium peroxide with water

1) Na₂O₂+H₂O→2NaOH+½O₂ formula 1

Hydrolysis reaction of potassium peroxide with water

2) K₂O₂+H₂O→2KOH+½O₂ formula 2

Hydrolysis reaction of calcium peroxide with water

3) Ca₂O₂+H₂O→Ca(OH)₂+½O₂ formula 3

Hydrolysis reaction of magnesium peroxide with water

4) MgO₂+H₂O→Mg(OH)₂+½O₂ formula 4

Hydrolysis reaction of sodium percarbonate with water

5) Na₂CO₃+H₂O₂+H₂O→2NaOH+H₂CO₃+½O₂ formula 5

When the control panel 600 supplies power to the motor 132a of the stirrer 132, the motor 132a rotates, the first bevel gear 132b installed at the end of the motor shaft rotates, the second bevel gear 132c engaged with the bevel gear 132b at an angle of 90 degrees rotates, the rotating shaft 132d connected to the second bevel gear 132c and the first quantum energy generating coil 133 installed at intervals in the circumferential direction of the outer surface of the rotating shaft 132d simultaneously rotate, and the mixed solution in which the oxygen generating raw material and the catalyst material are supplied to the municipal water or purified water is stirred and mixed by the stirrer 132 function of the rotating shaft 132d.

In addition, The DC power in the form of pulses is supplied from the power supply unit 135 of the first quantum energy generator to the first quantum energy generating coil 133 installed on the outer circumferential surface of the rotating shaft 132d and the second quantum energy generating coil 134 installed on the inner circumferential surface of the main body 131, a plurality of first quantum energy generating coils 133 installed on the outer surface of the rotating shaft 132d are installed through a roller bearing 135m installed on one lower side of the second bevel gear 132c.

The first quantum energy generating coil 133 installed at the same height to face the installation position of the second quantum energy generating coil 134, the winding directions of the coils are wound in opposite directions in the first quantum energy generating coil 133 and the second quantum energy generating coil 134.

The electromagnetic field of the pulsed electromagnetic field (PEMF) is generated in opposite directions at an angle of 90 degrees from the first quantum energy generating coil 133 and the second quantum energy generating coil 134, and applied to the mixed solution.

And at the same time, the electromagnetic fields in opposite directions overlap and offset (dissipate) at a central distance between the first quantum energy generating coil 133 and the second quantum energy generating coil 134, and pulsed quantum energy in the form of a pulse is generated in the zero magnetic field state, and an oxygen gas is generated in the main body by accelerating hydrolysis reaction with water while irradiating to the mixture.

In addition, when the oxygen-generating raw material and the catalyst material are introduced into the main body 131, they are precipitated at the lower portion of the main body 131 because the oxygen-generating raw material and the catalyst material are larger than the specific gravity of water, and thus the stirrer 132 is installed and stirred, so that the oxygen-generating raw material and the catalyst material are in sufficient contact with the municipal water, and the drain valve 139a installed on the exhaust line 139 is opened every predetermined time to small amounts periodically discharge the reaction by-product (NaOH, KOH, Ca(OH)₂, Mg(OH)₂, H₂CO₃) precipitated at the bottom of the main body 131.

Also, the hydrogen gas generated by the hydrolysis reaction is supplied to a mixer 154 using the FAN 137, in the mixer 154, any one inert gas selected from the inert gas supply unit 150 (N₂, CO₂, Ar) is mixed to be 96% or more of the total volume and less than 4% of the total volume of the hydrogen gas, and then supplied to the ion generator 200.

The power supply unit 135 for supplying the pulse type power includes a first rectifier 135a, a transformer 135b, a FET switch 135c, a second rectifier 135d, a pulse control unit 135e, a magnetic field detection sensor 135e-1, a switching control unit 135f, and a post-regulator 135g, a pulse control unit 135e includes functions of a pulse width modulation (PWM) control, a pulse frequency modulation (PFM) control, a pulse frequency (density) modulation (PDM) control, and a pulse repetition rate (PRR) control.

When an AC voltage is supplied to the first rectifier 135a, the AC voltage supplied from the first rectifier 135a is converted into a DC voltage, and the converted DC voltage is supplied to the transformer 135b.

The transformer 135b decompresses to a low voltage of 220V or less, or transforms to a high voltage in the range of 1 kV to 300 kV and supplies it to the FET switch 135c, the FET switch 135c supplies the AC voltage modulated to a high frequency generated by the high frequency switching to the second rectification unit 135d, the second rectification unit 135d converts the AC voltage modulated to a DC voltage, the converted DC voltage is supplied to the pulse control unit 135e, the pulse control unit 135e controls the pulse width modulation (PWM), the pulse frequency modulation (PFM), the pulse frequency (density) modulation (PDM), and the pulse repetition rate (PRR), etc. to supply the FET switch 135c. Also, the switching control unit 135f stops the high-frequency switching operation of the FET switch 135c before the transmission pulse is generated by the pulse control unit 135e.

The transmission pulse is generated by the pulse control unit 135e and simultaneously operated to supply the DC voltage to the first and second quantum energy generation coils 133 and 134.

In the first power supply unit 135, the frequency modulation range is in the range of 1 Hz to 100 Hz, 100 Hz to 1 kHz, 1 kHz to 10 kHz, 10 kHz to 1 MHZ, 1 MHz to 100 MHz, and 100 MHZ to 10 GHZ.

As a material of the exhaust fan 137 casing, one material is selected from among non-spark materials such as PVC, FRP (glass fiber molded foam), and carbon fiber molded foam, and as a material of the blade of the exhaust fan 137, one material is selected from among non-spark materials such as copper or copper alloy, duralumin or aluminum alloy, or FRP (glass fiber molded foam) is coated on a surface of a steel plate or stainless steel (STS304) material at a predetermined thickness or more to have the performance of non-spark materials.

The reason why the material of the casing and the blade of the exhaust fan 137 is selected and adopted as the non-spark material is that the motor of the exhaust fan 137 generates a spark in the case of a general metal material due to the collision and scratch phenomenon with the inner surface of the casing due to the separation or abnormal rotation of the blade connected to the rotating shaft inside the casing when the bearing into which the rotating shaft is inserted is worn out and damaged, and the casing and the blade are made of the non-spark material in order to prevent fire and explosion by the spark generated by the collision and scratch phenomenon of the combustible gas (H₂) passing through the casing of the exhaust fan 137.

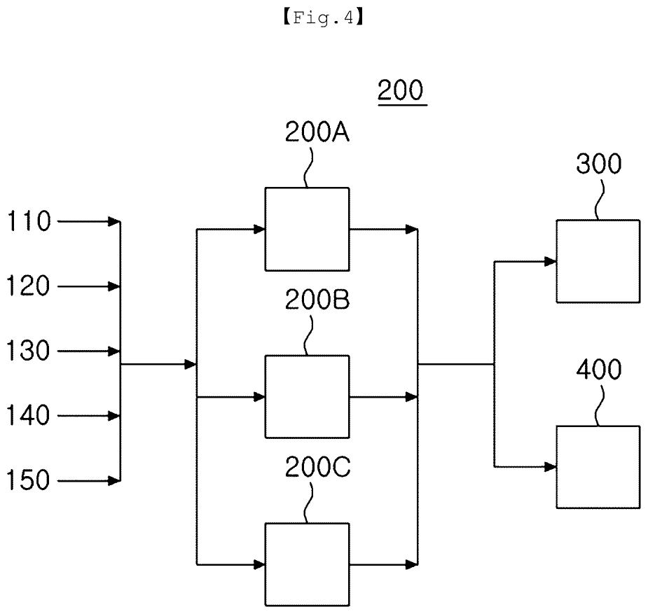

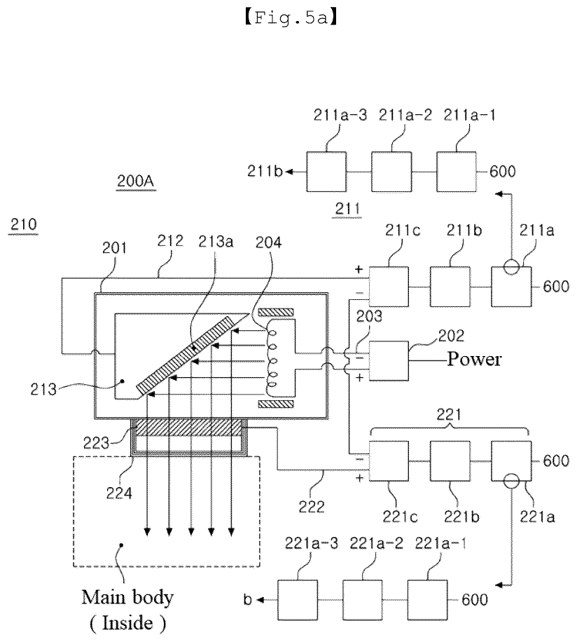

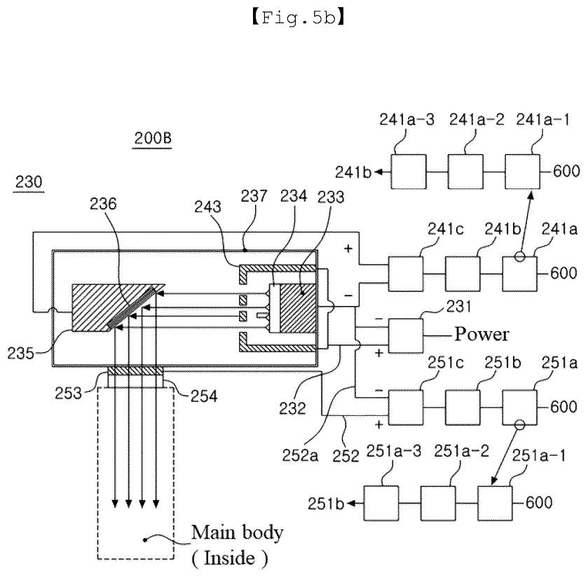

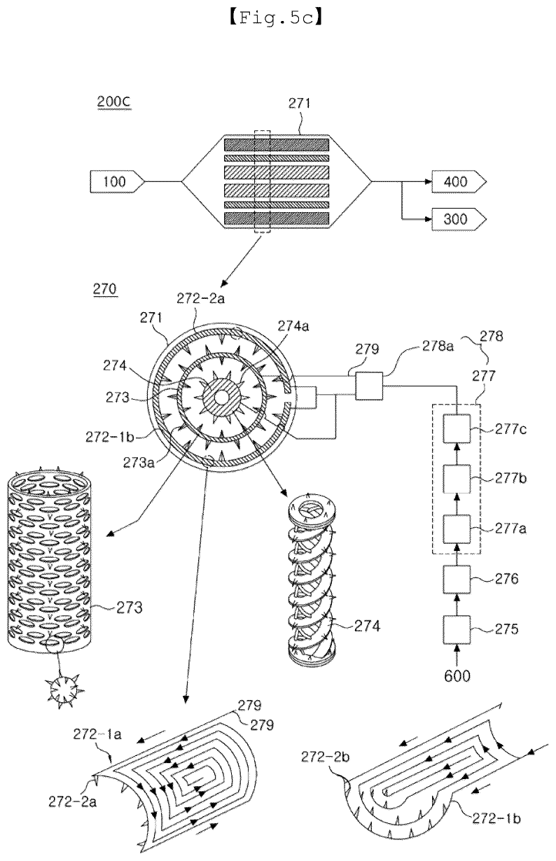

FIG. 4 is a schematic diagram illustrating the ion generator shown in FIG. 1. FIG. 5A is a cross-sectional view illustrating a first ion generator 200A of FIG. 4. FIG. 5B is a cross-sectional view illustrating a second ion generator 200B of FIG. 4. FIG. 5C is a cross-sectional view illustrating a third ion generator 200C of FIG. 4.

Referring to the attached drawings, the ion generator 200 includes generator 200A, and the first ion generator 200A is a first quantum energy generator 210 in the form of a brake radiation type consisting of a sealed glass tube 201, a thermionic emission cathode 204 having a filament therein, a DC power supply unit 202 for the cathode 204, a first anode 213 made of copper (Cu) or tungsten (W), a high voltage power supply unit 211 for the first anode 213, a second anode 223 made of a material such as rhodium, or the like, a DC high voltage power supply unit 221 for the second anode 223, and a quantum energy emission layer 224 made of a material such as beryllium or the like.

Referring to the attached drawings, the ion generator 200 includes a second ion generator 200B. The second ion generator 200B of a second quantum energy generator 230 type includes a sealed glass tube 237, a cathode 233 installed in a fixture (not shown) of a central portion therein, an electron emission source 234, a gate electrode 243, a first anode 235, an X-ray target plate 236, a second anode 253, a quantum energy emission layer 254, a first power supply unit 231 for the cathode 233 and the first anode 235, a second power supply unit 241 for the gate electrode 243 and a third power supply unit 251 for applying a high voltage for the second anode 253.

Referring to the attached drawings, the ion generator 200 includes a third ion generator 200C. The third ion generator 200C of the high voltage discharge with a third quantum energy generator 270 type includes a main body (a housing) 271, a first gradient saddle coil 272-1a having first discharge electrodes 272-2a, a second gradient saddle coil 272-1b having second discharge electrodes 272-2b, a modified Trigger coil 273 having a ground electrode 273a, a modified Trigger coil 274 having third discharge electrodes 274a, a high-voltage generator 278 and a conducting wire 279.

A dust-removed air provided from the first material supply unit 110, a oxygen gas decompressed from the second raw material supply unit 120 and provided at the optimum flow rate, hydrogen gas (H₂) and oxygen gas (O₂) generated and supplied by hydrolysis in the third material supply unit 130, a hydrogen gas decompressed from the fourth raw material supply unit 140 and provided at the optimum flow rate and any one of nitrogen (N₂), carbon dioxide (CO₂), and argon (Ar) gas decompressed from the inert gas supply unit 150 and provided at the optimum flow rate are supplied to the mixer 154, selectively.

The hydrogen gas supplied from the third raw material supply unit 130 and the fourth raw material supply unit 140 are mixed with the inert gas in the mixer 154 so that the hydrogen content is less than 4%, and supplied to the inside of the main body (not shown) of the ion generators 200A, 200B, and 200C.

The gases such as the air, the hydrogen, the oxygen, the carbon dioxide are supplied from the raw material supply unit 100 and passes through the inside of the main chamber, the quantum energy generated by the radiation moderation phenomenon is irradiated to the gases.

By quantum energy irradiation of the braking radiation type or quantum energy irradiation of the high-voltage discharge method, the covalent bond of the gas material is dissociated, and the gas material becomes an atom or a cation or an anion state. Then, the atoms (or ion) are supplied to a dissolving device 300 and ion separator 400.

FIG. 5A is a cross-sectional view illustrating a first ion generator 200A. Referring to the attached drawings, the first ion generator 200A includes a first quantum energy generator 210 of a brake radiation type, and a plurality of first ion generators 200A are installed on an outer surface of a main body (not shown) of the ion generator 200A.

Quantum energy is irradiated to air, oxygen (O₂), nitrogen (N₂), and hydrogen (H₂) gases introduced into the main body from the raw material supply unit 100.

In the first quantum energy generator 210, a thermoelectric emission cathode 204 having a filament is installed inside a left side surface of a sealed glass tube 201, a power supply unit 202 and a conducting wire 203 for supplying DC power to the cathode 204 are connected to the cathode 204, a first anode 213 made of copper (Cu) or tungsten (W) is installed to be spaced apart from the cathode 204 by a predetermined distance, a target plate 213a is installed to be in surface contact with a right inclined surface of the anode 213

A high voltage power source 211 for applying a high voltage to the first anode 213 is installed. Here, the high voltage power source 211 includes a variable voltage supply unit 211a, a rectifying circuit 211b, and a booster 211c for boosting the rectified DC power source, and the variable voltage supply unit 211a includes a control unit 211a-1 including a switching element, a first division resistor 211a-2, and a distribution circuit 211a-3.

The control unit 211a-1 of the variable voltage supply unit 211a includes a plurality of N-th ports (Cpu P1, Cpu P2, . . . , Cpu PN) that selectively output high/low signals.

Each of the first distribution resistors (211a-2) has first to Nth resistors (R1, R2, . . . , Rn) connected in series at the emitter side (not shown).

Each of the first to Nth ports (Cpu P1, Cpu P2, . . . /Cpu PN) of the control unit 211a-1 is connected to the base terminal and is connected in parallel to the first to Nth transistors (not shown) connected in parallel to each other.

The distribution circuit 211a-3 includes a second distribution resistor (not shown) connected in series to the first to N-th transistors (not shown) and outputs a voltage divided between the first distribution resistor and the second distribution resistor.

That is, the AC power supplied from the control panel 600 is changed (e.g., increased or decreased) by the variable voltage supply unit 211a, the variable AC power supplied from the variable voltage supply unit 211a is converted into the variable DC power DC by the rectifying circuit 211b, and the converted DC power DC is supplied to the first anode 213 after the voltage is increased to a high voltage by the booster 211c.

The second anode 223 made of a material such as rhodium is installed in an opening, which is opened in a predetermined area, at a lower portion of a central distance from the first anode 213 installed at one side of a left side while being spaced apart from the thermal electron emission cathode 204 by a predetermined distance inside the sealed glass tube 201. DC variable power generated by the DC power supply unit 221 including a variable voltage supply unit 221a, a rectification circuit 221b, and a booster 221c for boosting rectified DC power is supplied to the second anode 223 through a conducting wire.

A control unit 221a-1 of the variable voltage supply unit 221a includes N-th ports (Cpu P1, Cpu P2, . . . , Cpu PN) that selectively output high/low signals.

Each of the first distribution resistors 221a-2 has first to Nth resistors (R1, R2, . . . , Rn) connected in series at the emitter side (not shown).

Each of the first to Nth ports (Cpu P1, Cpu P2, . . . , Cpu PN) of the control unit 221a-1 is connected to the base terminal and is connected in parallel to the first to Nth transistors (not shown) connected in parallel to each other.

A distribution circuit 221a-3 includes a second distribution resistor (not shown) connected in series to the first to N-th transistors (not shown) and outputs a voltage divided between the first distribution resistor and the second distribution resistor.

That is, the AC power supplied from the control panel 600 is changed (e.g., increased or decreased) by the variable voltage supply unit 221a, the variable AC power supplied from the variable voltage supply unit 221a is converted into the variable DC power DC by the rectifying circuit 221b, and the converted DC power DC is supplied to the second anode 223 after the voltage is increased to a high voltage by the booster 221c.

In addition, a quantum energy emitting layer 224 made of a material such as beryllium is formed on an exposed surface of the second anode 223, power is supplied to the cathode 204 through the power supply unit 202, a conducting wire 212 connected to the first anode 213 is connected to an output side positive (+) terminal of the first anode power supply unit 211.

And a conducting wire 203 connected to an output side negative (−) terminal of the cathode power supply unit 202 is connected to an output side negative (−) terminal of the first anode power supply unit 211, so that a bias circuit is formed between the cathode power supply unit 202 and the first anode power supply unit 211, and a high voltage of 10 to 30 kV is applied between the first anode 213 and the cathode 204.

In addition, a high voltage of 20 to 50 kV is applied between the first anode 213 and the second anode 223.

In addition, the conducting wire 222 connected to the second positive electrode 223 is connected to the output side positive (+) terminal of the second anode power supply unit 221. And a conducting wire connected to an output side negative (−) terminal of the second anode power supply unit 221 is connected to an output side negative (−) terminal of the first anode power supply unit 211, so that a bias circuit is formed between the second anode power supply unit 221 and the first anode power supply unit 211, and a high voltage of 10 to 30 kV is applied between the first anode 213 and the second anode 223.

According to an embodiment of irradiating quantum energy to the main body space, power is supplied to the cathode 204 to generate thermoelectrons at the cathode 204, and the generated thermoelectrons are accelerated toward the first anode 213 by a bias circuit between the first anode power supply unit 211 and the cathode power supply unit 202, and strongly collide with a first anode (target) made of a tungsten (W) material due to electrical attraction. As a result, x-rays are generated.

At the same time, the high voltage generated by the second anode power supply unit 221 is supplied to the second anode 223 and the thermoelectrons collided with the target plate 213a accelerates in the direction of the second anode 223 by a bias circuit configured between the first anode power supply unit 211 and the second anode power supply unit 221, after being impacted by the collision with the extranucleus electrons of the second anode 223 material.

Then, a certain amount of kinetic energy is dissipated by attenuation while colliding with the nuclear extrinsic electrons (Extranuclear) of the second anode 223 material, and the residual energy generates X-ray and quantum energy having a wave.

The X-ray and quantum energy penetrating the second anode 223 are converted into photoelectron and quantum energy with waves as they are irradiated to the emission layer 224 of non-radial matter, and the quantum energy generated by the radiation moderation phenomenon is irradiated inside of the main chamber.

Moreover, the material of the sealed glass tube 201 is the hard glass. And the degree of vacuum of the sealed glass tube 201 is 10⁻⁶ to 10⁻⁸ mmHg range

As the material of the cathode 204, any one material is selected and used from tungsten or a material containing tungsten, rhenium, tantalum, and the like.

In another embodiment, the material of the anode 204 may be selected from one of carbon nanotubes, carbon nanofibers (CNF), nano-wire, graphene, and nanodiamond.

According to an embodiment of irradiating quantum energy to the main body space, power is supplied to the cathode 204 to generate thermoelectrons at the cathode 204, and the generated thermoelectrons are accelerated toward the first anode 213 by a bias circuit between the first anode power supply unit 211 and the cathode power supply unit 202, and strongly collide with the target plate 213a installed in a surface-contact manner with an inclined surface of the first anode 213 of a tungsten (W) material due to electrical attraction. As a result, x-rays are generated.

At the same time, the high voltage generated by the second anode power supply unit 221 is supplied to the second anode 223 and the thermoelectrons collided with the target plate 213a accelerates in the direction of the second anode 223 by a bias circuit configured between the first anode power supply unit 211 and the second anode power supply unit 221, after being impacted by the collision with the extranucleus electrons of the second anode 223 material.

Then, a certain amount of kinetic energy is dissipated by attenuation while colliding with the nuclear extrinsic electrons (Extranuclear) of the second anode 223 material, and the residual energy generates X-ray and quantum energy having a wave.

The X-ray and quantum energy penetrating the second anode 223 are converted into photoelectron and quantum energy with waves as they are irradiated to the emission layer 224 of non-radial matter, and the quantum energy generated by the radiation moderation phenomenon is irradiated inside of the main chamber.

The gases such as an air, a hydrogen, an oxygen, a nitrogen pass through the inside of the main chamber, the quantum energy generated by the radiation moderation phenomenon is irradiated to the gases. By quantum energy irradiation, the covalent bonds of the gases material are dissociated, and the gases material may become atoms, cations or anions.

In addition, the first anode power supply unit 211 including the variable voltage supply unit 211a for supplying power to the first anode 213, the rectifier circuit 211b, and the booster 211c for boosting the rectified DC power is configured to generate a variable DC high voltage of which a voltage is adjusted.
and the second anode power supply unit 221 including the variable voltage supply unit 221a for supplying power to the second anode 223, the rectifier circuit 221b, and the booster 221c for boosting the rectified DC power is configured to generate a variable DC high voltage of which a voltage is adjusted.

Thus, by adjusting the strength of the variable DC high voltage of the first anode power supply unit 211 and the second anode power supply unit 221, a power supply unit 202 for the cathode 204, thereby adjusting an acceleration force of thermoelectrons accelerated to the target plate 213a which is emitted from the cathode 204 and installed in surface contact with the first anode 213, thereby adjusting an amount of quantum energy and X-rays generated by strong collision with each other by an electrical attraction at the target plate 213a.

Also, it can be controlled to suit the covalent dissociation energy of air, oxygen, nitrogen, and hydrogen gases passing through the interior of the main chamber.

By the above process, the covalent bond of the air, the oxygen, the nitrogen, and the hydrogen molecule introduced into the main chamber is dissociated and it supplies to the dissolving device 300 and the ion separator 400 to the atom (or, the ion) state.

FIG. 5B is a cross-sectional view illustrating the second ion generator 200B. Referring to the attached drawings, the second ion generator 200B includes the second quantum energy generator 230 of the braking radiation type, and a plurality of second ion generators 200B may be installed on an outer surface of a main chamber (not shown).

The quantum energy generated from the second quantum energy generator 230 is irradiated to the air, the oxygen (O₂), the nitrogen (N₂), and the hydrogen (H₂) introduced from the raw material supply unit 100 into the main chamber.

In the second quantum energy generator 230, a negative electrode 233 is installed in a fixture (not shown) at a central portion inside a right side surface of a sealed glass tube 237, an electron emission source 234 is installed in a surface contact with the negative electrode 233, and a gate electrode 243 is installed in a shape spaced apart from the negative electrode 233 by a predetermined distance in a left direction to enclose the negative electrode 233.

A first anode 235 having an inclined right side surface is installed at the center of the left side surface inside the sealing glass tube 237 to face the gate electrode 243, and the X-ray target plate 236 is installed in surface contact with the right inclined surface of the first anode 235.

In addition, a second anode 253 is installed on the sealing glass tube 237 at a portion vertically projected downward on the target plate 436 provided on the inclined surface of the first anode 235, and a quantum energy emitting layer 254 is installed on the surface of the second anode 253 in a surface-contact manner.

In addition, a first power supply unit 231 for applying a high voltage to the cathode 233 and the first anode 235 by being spaced apart from the left side surface of the sealing glass tube 237 by a predetermined distance through a conducting wire is installed, and the second power supply unit 241 is installed to supply high voltage DC power to the gate electrode 243.

The second power supply unit 241 includes a variable voltage supply unit 241a, a rectifying circuit 241b, and a booster 241c for boosting the rectified DC power. Also, the variable voltage supply unit 241a includes a control unit 241a-1, a first distribution resistor 241a-2, and a distribution circuit 241a-3.

The second power supply unit 241 may be a high voltage power supply unit 241 to apply high voltage.

The controller 241a-1 of the variable voltage supply unit 241a includes a plurality of N-th ports (Cpu P1, Cpu, P2, . . . , Cpu PN) that selectively output high/low signals.

The first distribution resistor 241a-2 has first to N-th resistors R1, R2, . . . , Rn connected in series to each other at the emitter side (not shown), and base terminals of the first to N-th ports (Cpu P1, Cpu P2, . . . , Cpu PN) of the controller 241a-1 are connected in parallel to the first to N-th transistors (not shown) connected in parallel to each other.

The distribution circuit 241a-3 includes a second distribution resistor (not shown) connected in series to the first to N-th transistors and outputs a voltage divided between the first distribution resistor and the second distribution resistor.

That is, the AC power source AC supplied from the control panel 600 is changed (e.g., increased or decreased) in the variable voltage supply unit 241a, the variable AC power source AC supplied from the variable voltage supply unit 241a is converted into the variable DC power source DC in the rectifying circuit 241b, and the converted DC power source DC is supplied to the first anode 235 after the voltage is increased to a high voltage in the booster 241c.

A bias circuit between the first power supply unit 231 and the second power supply unit 241 is formed by wiring the output negative (−) terminals of the first power supply unit 231 and the second power supply unit 241 in common.

The third power supply unit 251 is installed in a downward direction from the first power supply unit 231 to supply high voltage DC power to the second anode 253. The third power supply unit 251 includes a variable voltage supply unit 251a, a rectification circuit 251b and a booster 251c for boosting rectified DC power, the variable voltage supply unit 251a includes a control unit 251a-1, a first distribution resistor 251a-2, and a distribution circuit 251a-3.

The controller 251a-1 of the variable voltage supply unit 251a includes a plurality of N-th ports (Cpu P1, Cpu P2, . . . , Cpu PN) that selectively output high/low signals.

The first distribution resistor 251a-2 has first to N-th resistors R1, R2, . . . , Rn connected in series to each other at the emitter side (not shown), and base terminals of the first to N-th ports (Cpu P1, Cpu P2, . . . , Cpu PN) of the controller 251a-1 are connected in parallel to the first to N-th transistors (not shown) connected in parallel to each other.

The distribution circuit 251a-3 includes a second distribution resistor (not shown) connected in series to the first to N-th transistors and outputs a voltage divided between the first distribution resistor and the second distribution resistor.

That is, the AC power source AC supplied from the control panel 600 is changed (e.g., increased or decreased) in the variable voltage supply unit 251a, the variable AC power source AC supplied from the variable voltage supply unit 251a is converted into the variable DC power source DC in the rectifying circuit 251b, and the converted DC power source DC is supplied to the second anode 253 after the voltage is increased to a high voltage in the booster 251c.

Also, a bias circuit between the second power supply unit 241 and the third power supply unit 251 is formed by wiring the output negative (−) terminals of the second power supply unit 241 and the third power supply unit 251 in common.

When a high voltage in a range of 10 kV to 30 kV generated in a first power supply unit 231 is applied through a conducting wire 232 to the first anode 235 and the cathode 233 in which the electron emission source 234 is arranged, a high potential difference is formed between the cathode 233 in which the electron emission source 234 is arranged and the first anode 235.

Therefore, the first anode 235 serves as an acceleration electrode, and simultaneously serves as an X-ray target which emits X-rays by collision of accelerated electrons emitted from the electron emission source 234.

To this end, the first anode 235 has an X-ray target plate 236 inclined obliquely with respect to a direction in which an electron beam travels inside the sealed glass tube 201. A separate target member may be installed on the target plate 236. The target member may include any one of tungsten (W), copper (Cu), molybdenum (Mo), cobalt (Co), chromium (Cr), iron (Fe), silver (Ag), tantalum (Tr), yttrium (Y), etc. which emit X-rays by hitting an accelerated electron beam (E). The material of tungsten (W) having a high melting point and excellent X-ray emission efficiency may be mainly used.

The gate electrode 243 receiving the power generated by the second power supply unit 241 to the conducting wire 242 is spaced apart from the first anode 235 by a predetermined distance in the right direction, and is installed close to the electron emission source 234 installed in surface contact with the surface of the cathode 233 to surround the electron emission source 234 and the cathode 233 at intervals.

The gate electrode 234 may include a plurality of gate holes through which the braille beam passes. The gate electrode 234 may include the form of a horizontal electrode having the form of a mesh net, a perforated thin metal perforated plate, or a metal mesh.

When a high voltage is applied to the first power supply unit 231 and electrons accelerated by a high voltage applied to the first anode 235 collide with the X-ray target plate 236 of the first anode 235, some energy is emitted in the form of X-rays, but more energy is converted into heat, so that it may be overheated. Therefore, the heat capacity of the first anode is increased to facilitate self-thermal diffusion, thereby preventing rapid temperature increase.

The second anode 253 is connected to the power supply unit 251 for supplying DC high voltage power through a conducting wire 252, and the quantum energy emission layer 254 made of a material such as beryllium is installed on an exposed surface of the second anode 223, and a high voltage of 20 to 50 kV is applied between the first anode 235 and the second anode 253.

In addition, the second anode 253 is connected to the output side+terminal of the second anode power supply unit 251 through a conducting wire 252, and a conducting wire 252a connected to an output side negative (−) terminal of the second anode power supply unit 251 is connected to an output side negative (−) terminal of the first anode power supply unit 241, so that a bias circuit is formed between the first anode power supply unit 241 and the second anode power supply unit 251, and a high voltage of 20 to 50 kV is applied between the first anode 235 and the second anode 253.

In addition, the first anode power supply unit 241 including the variable voltage supply unit 241a for supplying power to the first anode 235, the rectifier circuit 241b, and the booster 241c for boosting the rectified DC power is configured to generate a variable DC high voltage of which a voltage is adjusted.
and the second anode power supply unit 251 including the variable voltage supply unit 251a for supplying power to the second anode 253, the rectifier circuit 251b, and the booster 251c for boosting the rectified DC power is configured to generate a variable DC high voltage of which a voltage is adjusted.

Thus, by adjusting the strength of the variable DC high voltage of the first anode power supply unit 241 and the second anode power supply unit 251, the power supply unit 231 for the cathode 233 and the gate electrode 243, thereby adjusting an acceleration force of thermoelectrons accelerated to the target plate 236 which is emitted from the cathode 233 and installed in surface contact with the first anode 235, thereby adjusting an amount of quantum energy and X-rays generated by strong collision with each other by an electrical attraction at the target plate 236.

Also, it can be controlled to suit the covalent dissociation energy of air, oxygen, nitrogen, and hydrogen gases passing through the interior of the body.

Moreover, the material of the sealed glass tube 237 is the hard glass.

And the degree of vacuum of the sealed glass tube 237 is 10⁻⁶ to 10⁻⁸ mmHg range.

And the degree of vacuum of the sealed glass tube 237 is 10⁻⁶ to 10⁻⁸ mmHg range.

As the material of the cathode 233, any one material is selected and used from tungsten or a material containing tungsten, rhenium, tantalum, and the like.

In another embodiment, the material of the anode 233 may be selected from one of carbon nanotubes, carbon nanofibers (CNF), nano-wire, graphene, and nanodiamond.

According to an embodiment of irradiating quantum energy to the main body space,

By supplying power to the cathode 233, electrons are generated from the electron emitting source 234 installed to be in contact with the cathode 233, the generated electrons are accelerated toward the first anode 235 by a high potential difference (10 to 30 kV) formed between the first anode 235 and the electron emission source 234 to collide with the target 236 installed in a surface-contact manner with the inclined surface of the first anode 236 made of tungsten (W) to generate X-rays.

At the same time, the high voltage generated by the second anode power supply unit 251 is supplied to the second anode 253 and the electrons collided with the target plate 236 accelerates in the direction of the second anode 253 by a bias circuit configured between the first anode power supply unit 241 and the second anode power supply unit 251, after being impacted by the collision with the extranucleus electrons of the second anode 253 material.

Then, a certain amount of kinetic energy is dissipated by attenuation while colliding with the nuclear extrinsic electrons (extranuclear) of the second anode 253 material, and the residual energy generates X-ray and quantum energy having a wave.

The X-ray and quantum energy penetrating the second anode 253 are converted into photoelectron and quantum energy with waves as they are irradiated to the emission layer 254 of non-radial matter, and the quantum energy generated by the radiation moderation phenomenon is irradiated inside of the main chamber.

Moreover, the material of the sealed glass tube 237 is the hard glass, and the degree of vacuum of the sealed glass tube 237 is 10⁻⁶ to 10⁻⁸ mmHg range.

The electron emission source 234 is installed on the cathode 233, the electron emission source 234 may be provided on a separate substrate to be coupled to the cathode 233, or may be directly formed on the surface of the cathode 233.

In addition, the electron emission source 234 may be formed by using a chemical vapor deposition method to directly grow a plurality of carbon nanotubes on the surface of the substrate or the cathode 233, or the electron emission source 234 may be formed by applying a carbon nanotube paste to the surface of the substrate or the cathode 233 and then firing the carbon nanotube paste. A material of the substrate or the cathode 233 may be any one of carbon nanotubes, carbon nano-fibers, nano-wires, graphene, and nano-diamonds.

By supplying power to the cathode 233 of a second quantum energy generation device 230, electrons are generated from the electron emitting source 234 installed to be in contact with the cathode 233, the generated electrons are accelerated toward the first anode 235 by a bias circuit between a second power supply unit 241 for the first anode 235 and the first power supply unit 231 for the cathode 233, to collide with the target 236 installed in a surface-contact manner with the inclined surface of the first anode 236 made of tungsten (W) to generate X-rays.

At the same time, the high voltage generated by the second anode power supply unit 251 is supplied to the second anode 253 and the electrons collided with the target plate 236 accelerates in the direction of the second anode 253 by a bias circuit configured between the first anode power supply unit 241 and the second anode power supply unit 251, after being impacted by the collision with the extranucleus electrons of the second anode 253 material.

Then, a certain amount of kinetic energy is dissipated by attenuation while colliding with the nuclear extrinsic electrons (extranuclear) of the second anode 253 material, and the residual energy generates X-ray and quantum energy having a wave.

The X-ray and quantum energy penetrating the second anode 253 are converted into photoelectron and quantum energy with waves as they are irradiated to the emission layer 254 of non-radial matter, and the quantum energy generated by the radiation moderation phenomenon is irradiated inside of the main chamber.

By the above process, the covalent bond of the air, the oxygen, the nitrogen, and the hydrogen molecule introduced into the main chamber is dissociated and it supplies to the dissolving device 300 and the ion separator 400 to the atom (or, the ion) state.

FIG. 5C is a cross-sectional view illustrating the third ion generator 200C (270) of FIG. 4. Referring to the accompanying drawings, the third ion generator 200C (270) of high-voltage discharge method includes a main body (a housing) 271, a first gradient coil 272-1a having first discharge electrodes 272-2a, a second gradient saddle coil 272-1b having second discharge electrodes 272-2b, a modified Trigger coil 273 having a ground electrode 273a, a modified Trigger coil 274 having third discharge electrodes 274a, a high-voltage generator 278 and conducting wires.

The first gradient saddle coil 272-1a having the first discharge electrode 272-2a is applied with an insulating material on the circumferential surface of the inner upper portion of the main body 271, and is installed on the upper side by being in surface contact with the insulating layer after the insulating treatment, and the second gradient saddle coil 272-1b having the second discharge electrode 272-2b is applied with an insulating material on the circumferential surface of the inner lower portion of the main body 271 and is installed on the lower side by being in surface contact with the insulating layer after the insulating treatment, so as to face the first gradient saddle coil 272-1a having the first discharge electrode 272-2a.

The second gradient saddle coil 272-1b having the second discharge electrode 272-2b is wound in a direction opposite to the winding direction of the first gradient saddle coil 272-1a having the first discharge electrode 272-2a.

In addition, a plurality of first discharge electrode 272-2a processed into a pointed shape (needle-shaped structure) having a predetermined length at intervals are provided on one side (opposite direction in contact with the main body) of the first gradient saddle coil 272-1a, and a plurality of second discharge electrode 272-2b processed into a pointed shape (needle-shaped structure) at intervals are provided on one side (opposite direction in contact with the main body) of the second gradient saddle coil 272-1b.

The modified Trigger coil 273 having the ground electrode 273a has a cylindrical shape (cylindrical shape) of a hollow structure, and a plurality of elliptical holes are formed in the circumferential direction of the outer surface. A plurality of ground electrode 273a processed into a pointed shape (needle-shaped structure) having a predetermined length at intervals are installed on inner side and outer side of the non-perforated circumferential surface at intervals in the circumferential direction, and are installed on a fixing plate (not shown) at intervals in a concentric circle formed by the first gradient saddle coil 272-1a having the first discharge electrode 272-2a and the second gradient saddle coil 272-1b having the second discharge electrode 272-2b.

The modified Trigger coil 274 having the third discharge electrode 274a with multiple discharge needles installed inside the modified Trigger coil 273, which is processed into a spiral screw shape at intervals inside the modified Trigger coil 273, and has a pointed shape (needle-shaped structure) at circumferential intervals at the screw edge.

A positive (+) terminal on the output side of the power supply unit 278 is connected to the first inclined saddle coil 272-1a having the first discharge electrode 272-2a, the second inclined saddle coil 272-1b having the second discharge electrode 272-2b, and the modified Trigger coil 274 having the third discharge electrode 274a. A negative (−) terminal on the output side of the power supply unit 278 is connected to the modified Trigger coil 273 having the ground electrode 273a.

The power supply unit 278 for generating pulsed electromagnetic field (PEMF) type power includes a booster 275, a rectifier circuit 276, a control unit 277 and a switching element 278a, the control unit 277 includes an input module 277a, a calculation module 277b, and a control module 277c having a pulse width modulation (PWM) control method, a pulse frequency modulation (PFM), a pulse frequency (density) control (PDM), and a pulse repetition rate control (PRR) function.

When AC power having a single phase of 220 V and 60 Hz is supplied to the booster 275, the booster 275 boosts the AC power to AC power having a single phase of 10 kV to 500 kV and 1 kHz to 300 kHz, and the rectifier circuit 276 converts the AC power into DC power.

The input module 277a of the control unit 277 inputs, to a program embedded therein, variables such as a current value, a voltage value, a pulse width, a pulse density pulse period, a frequency burst length, a main power supply time, a stop time (timer function), a switching element function, and the like supplied to the first and second quantum energy generation coils 272-1a and 272-1b, that is, the minimum (mA) and the maximum value (A) of the variable current flowing through the first and second quantum energy generation coils 272-1a and 272-1b, the minimum value (mT) and the maximum value (T) of the variable magnetic field, the power supply time (seconds to minutes, or minutes to hours) for each step, and the like. Each of the parameters may be set on the monitor (not shown) of the input module 277a, for example, the operation step among the steps divided into steps 1 to 10, and then data of the selected step may be input.

The operation module 277b may execute an operation program on a plurality of parameters input by the user to the input module 277a to calculate the first gradient saddle coil 272-1a having the first discharge electrode 272-2a for generating variable quantum energy, the second gradient saddle coil 272-1b having the second discharge electrode 272-2b for generating variable quantum energy, the modified Trigger coil 274 having the third discharge electrode 274a, and a current amount required for arc discharge and a current amount corresponding to a magnetic field strength to be generated by the modified Trigger coil having the ground electrode 273. The operation module 277b may generate corresponding data in advance to generate a current value in the form of one-to-one matching between the magnetic field strength value and the current value. For example, when the intensity value of the magnetic field is between 1 and 10, data about a current value corresponding to between 1 and 10 is generated, and when the intensity value of the magnetic field is 1, a power of a pulsed electromagnetic field (PEMF) type having a current value corresponding to 1 is generated.

The power of a pulsed electromagnetic field (PEMF) type having a current value corresponding to 1 is supplied to the first gradient saddle coil 272-1a having the first discharge electrode 272-2a, the second gradient saddle coil 272-1b having the second discharge electrode 272-2b, the modified Trigger coil having the ground electrode 273, and the modified Trigger coil having the discharge electrode 274.

The first gradient saddle coil 272-1a having the first discharge electrode 272-2a, the second gradient saddle coil 272-1b having the second discharge electrode 272-2b are wound in opposite winding directions and are installed up and down or on the left and right inside the main body.

A current flow of the first gradient saddle coil 272-1a proceeds multiple times from the right to the left direction, from the left to the semicircle direction, from the semicircle to the right direction, and from the right to the semicircle direction. At the same time, a high voltage is applied to the protruding first discharge electrode 272-2a.

A current flow of the second gradient saddle coil 272-1b proceeds multiple times from the right to the left direction, from the left to the semicircle direction, from the semicircle to the right direction, from the right to the semicircle direction, and from the semicircle to the left direction. At the same time, the high voltage is applied to the protruding second discharge electrode 272-2b.

At this time, the pulsed electromagnetic fields (PEMF) are irradiated from the first gradient saddle coil 272-1a and the second gradient saddle coil at an angle of 90 degrees in the current flow direction.

In other words, the pulsed electromagnetic fields (PEMF) is irradiated from top to bottom direction in the first gradient saddle coil 272-1a, and is irradiated from bottom to top direction in the second gradient saddle coil 272-1b.

As a result, the pulsed electromagnetic field (PEMF) in opposite directions overlap and offset (dissipate) in the center of the main body 271, creating in pulsating quantum energy in a zero magnetic field state, and the pulsating quantum energy is irradiated to the molecules of air, oxygen (O₂), nitrogen (N₂), and hydrogen (H₂) flowing into the main body 271.

At the same time, discharge begins between the modified Trigger coil 273 having the ground electrode 273a and the modified Trigger coil 274 having the third discharge electrode 274a, forming a high field electron energy band, and then processing this series of quantum energy irradiation and discharging on air, oxygen, nitrogen, and hydrogen molecules passing through the high field electron energy band.

At this time, the current detection sensor 278a installed on the lead-in line of the electrodes detects the current values flowing through the first gradient coil 272-1a having the first discharge electrode 272-2a, the second gradient saddle coil 272-1b having the second discharge electrode 272-2b, the modified Trigger coil 273 having the ground electrode 273a and the modified Trigger coil 274 having the third discharge electrode 274a in real time and transmits them to the calculation unit (feedback).

Then, the calculation unit 277b compares and analyzes the current value calculated and set for each variable input from the input unit 277a with the current value transmitted from the current detection sensor 278a, and analyzes the difference value when it deviates from the lower and upper limits of the set current value. After that, it is recalculated to suit the lower limit and upper limit, and then the modified current value is transmitted to the current control module 277c.

The current control module 277c receiving the modified current value supplies DC power to the first gradient coil 272-1a having the first discharge electrode 272-2a, the second gradient saddle coil 272-1b having the second discharge electrode 272-2b, the modified Trigger coil 273 having the ground electrode 273a and the modified Trigger coil 274 having the third discharge electrode 274a according to the modified current value.

As a result, pulsation quantum energy is investigated in the main body, and the covalent bonds of air, oxygen, nitrogen, and hydrogen molecules flowing in the main body are dissociated by an electrochemical reaction by high voltage discharge, and supplied to the dissolving device 300 and the ion separator 400 in an atomic (or ion) state.

In addition, the first and second inclined saddle coils 272-1a and 272-1b have the same or similar structure as Uniform saddle coil, so that the first and second inclined saddle coils 272-1a and 272-1b may be replaced with uniform saddle coils.

Accordingly, another configuration of the third ion generator 270 having a built-in discharge function may include a first uniform saddle coil 272-1a having first discharge electrodes 272-2a, a second uniform saddle coil 272-1b having second discharge electrodes 272-2b, a modified Trigger coil 273 having a ground electrode 273a, a modified Trigger coil 274 having third discharge electrodes 274a, a high-voltage generator 278 and conducting wires 279.

The first uniform saddle coil 272-1a having the first discharge electrode 272-2a is applied with an insulating material on the circumferential surface of the inner upper portion of the main body 271, and is installed on the upper side by being in surface contact with the insulating layer after the insulating treatment, and the second uniform saddle coil 272-1b having the second discharge electrode 272-2b is applied with an insulating material on the circumferential surface of the inner lower portion of the main body 271 and is installed on the lower side by being in surface contact with the insulating layer after the insulating treatment, so as to face the first uniform saddle coil 272-1a having the first discharge electrode 272-2a.

The winding direction of the second uniform saddle coil 272-1b having the second discharge electrode 272-2b and the first uniform saddle coil 272-1a having the first discharge electrode 272-2a and the current flow direction are the same as each other.

In addition, a plurality of first discharge electrode 272-2a processed into a pointed shape (needle-shaped structure) having a predetermined length at intervals are provided on one side (opposite direction in contact with the main body) of the first uniform saddle coil 272-1a, and a plurality of second discharge electrode 272-2b processed into a pointed shape (needle-shaped structure) at intervals are provided on one side (opposite direction in contact with the main body) of the second uniform saddle coil 272-1b.

The modified Trigger coil 273 having the ground electrode 273a has a cylindrical shape (cylindrical shape) of a hollow structure, and a plurality of elliptical holes are formed in the circumferential direction of the outer surface.

A plurality of ground electrode 273a processed into a pointed shape (needle-shaped structure) having a predetermined length at intervals are installed on inner side and outer side of the non-perforated circumferential surface at intervals in the circumferential direction, and are installed on a fixing plate (not shown) at intervals in a concentric circle formed by the first uniform saddle coil 272-1a having the first discharge electrode 272-2a and the second uniform saddle coil 272-1b having the second discharge electrode 272-2b.

The modified Trigger coil 274 having the third discharge electrode 274a with multiple discharge needles installed inside the modified Trigger coil 273, which is processed into a spiral screw shape at intervals inside the modified Trigger coil 273, and has a pointed shape (needle-shaped structure) at circumferential intervals at the screw edge.

The power supply unit 278 for generating pulsed electromagnetic field (PEMF) type power includes a booster 275, a rectifier circuit 276, a control unit 277 and a switching element 278a, the control unit 277 includes an input module 277a, a calculation module 277b, and a control module 277c having a pulse width modulation (PWM) control method, a pulse frequency modulation (PFM), a pulse frequency (density) control (PDM), and a pulse repetition rate control (PRR) function.

When AC power having a single phase of 220 V and 60 Hz is supplied to the booster 275, the booster 275 boosts the AC power to AC power having a single phase of 10 kV to 500 kV and 1 kHz to 300 kHz, and the rectifier circuit 276 converts the AC power into DC power.

The input module 277a of the control unit 277 inputs, to a program embedded therein, variables such as a current value, a voltage value, a pulse width, a pulse density pulse period, a frequency burst length, a main power supply time, a stop time (timer function), a switching element function, and the like supplied to the first uniform saddle coil 272-1a the first discharge electrode 272-2a, the second having uniform saddle coil 272-1b having the second discharge electrode 272-2b, the modified Trigger coil 273 having the ground electrode 273a and the modified Trigger coil 274 having the third discharge electrode 274a,
that is, the minimum (mA) and the maximum value (A) of the variable current flowing through the coils (272-1a, 272-1b, 273, 274), the minimum value (mT) and the maximum value (T) of the variable magnetic field, the power supply time (seconds to minutes, or minutes to hours) for each step, and the like. Each of the parameters may be set on the monitor (not shown) of the input module 277a, for example, the operation step among the steps divided into steps 1 to 10, and then data of the selected step may be input.

The operation module 277b may execute an operation program on a plurality of parameters input by the user to the input module 277a to calculate required current of the first uniform saddle coil 272-1a having the first discharge electrode 272-2a for generating pulsed electromagnetic fields, and the second uniform saddle coil 272-1b having the second discharge electrode 272-2b for generating pulsed electromagnetic fields, and a current amount required for arc discharge and a current amount corresponding to a magnetic field strength.

The operation module 277b may generate corresponding data in advance to generate a current value in the form of one-to-one matching between the magnetic field strength value and the current value. For example, when the intensity value of the magnetic field is between 1 and 10, data about a current value corresponding to between 1 and 10 is generated, and when the intensity value of the magnetic field is 1, a power of a pulsed electromagnetic field (PEMF) type having a current value corresponding to 1 is generated.

The power of a pulsed electromagnetic field (PEMF) type having a current value corresponding to 1 is supplied to the first uniform saddle coil 272-1a having the first discharge electrode 272-2a, the second uniform saddle coil 272-1b having the second discharge electrode 272-2b, the modified Trigger coil having the ground electrode 273, and the modified Trigger coil having the discharge electrode 274.

The first uniform saddle coil 272-1a having the first discharge electrode 272-2a, the second uniform saddle coil 272-1b having the second discharge electrode 272-2b are wound in same winding directions and are installed up and down inside the main body. The current flows of the first uniform saddle coil 272-1a and the second uniform saddle coil 272-1b proceed multiple times from the right to the left direction, from the left to the semicircle direction, from the semicircle to the right direction, and from the right to the semicircle direction. At the same time, a high voltage is applied to the protruding first discharge electrode 272-2a and the protruding second discharge electrode 272-2b

At this time, the pulsed electromagnetic fields (PEMF) in the same direction are irradiated from the first uniform saddle coil 272-1a and the second uniform saddle coil at an angle of 90 degrees in the current flow direction.

In other words, the pulsed electromagnetic fields (PEMF) is irradiated from bottom to top direction in the first uniform saddle coil 272-1a and the second uniform saddle coil 272-1b. As a result, the pulsed electromagnetic field (PEMF) in top directions is irradiated in the main body 271, and is irradiated to the molecules of air, oxygen (O₂), nitrogen (N₂), and hydrogen (H₂) flowing into the main body 271.

At the same time, discharge begins between the modified Trigger coil 273 having the ground electrode 273a and the modified Trigger coil 274 having the third discharge electrode 274a, forming a high field electron energy band, and then processing this series of quantum energy irradiation and discharging on air, oxygen, nitrogen, and hydrogen molecules passing through the high field electron energy band.

At this time, the current detection sensor 278a installed on the lead-in line of the electrodes detects the current values flowing through the first uniform coil 272-1a having the first discharge electrode 272-2a, the second uniform saddle coil 272-1b having the second discharge electrode 272-2b, the modified Trigger coil 273 having the ground electrode 273a and the modified Trigger coil 274 having the third discharge electrode 274a time and transmits them to the calculation unit (feedback).

Then, the calculation unit 277b compares and analyzes the current value calculated and set for each variable input from the input unit 277a with the current value transmitted from the current detection sensor 278a, and analyzes the difference value when it deviates from the lower and upper limits of the set current value. After that, it is recalculated to suit the lower limit and upper limit, and then the modified current value is transmitted to the current control module 277c.

The current control module 277c receiving the modified current value supplies DC power to the first uniform coil 272-1a having the first discharge electrode 272-2a, the second uniform saddle coil 272-1b having the second discharge electrode 272-2b, the modified Trigger coil 273 having the ground electrode 273a and the modified Trigger coil 274 having the third discharge electrode 274a according to the modified current value.

As a result, pulsation quantum energy is investigated in the main body, and the covalent bonds of air, oxygen, nitrogen, and hydrogen molecules flowing in the main body are dissociated by an electrochemical reaction by high voltage discharge. During the high-voltage discharge process, electrons having a negative charge are separated from the hydrogen molecule in the hydrogen molecule, so that hydrogen ions (H⁺) are generated, and the separated electrons are combined with other hydrogen molecules to form hydrogen ions (H⁻).

The hydrogen ions (that is, H⁺ and H⁻) charged to the positive charge and negative charge are attracted the neutral hydrogen molecules (H₂). At the same time, the hydrogen ions (that is, H⁺ and H⁻) are attracted to each other, causing a reduction phenomenon.

Moreover, among the high-voltage discharge process

Electrons having a negative charge are separated from the oxygen molecule, oxygen ions (O⁺) are generated, and the separated electrons are combined with other oxygen molecules to form other oxygen ions (O⁻) is created.

The oxygen ions (i.e. O⁺ and O⁻) charged to the positive charge and negative charge are attracted the neutral oxygen molecules (O₂). At the same time, the oxygen ions are attracted to each other, causing a reduction phenomenon.

In addition, the first uniform coil 272-1a having the first discharge electrode 272-2a, the second uniform saddle coil 272-1b having the second discharge electrode 272-2b, the modified Trigger coil 273 having the ground electrode 273a and the modified Trigger coil 274 having the third discharge electrode 274a are coated with a catalytic material (not shown) selected from one or more of titanium dioxide (TiO₂), rhodium (Rh), platinum (Pt), palladium (Pd), ruthenium, zinc (Zn), zirconium (Zr), hafnium, vanadium (V₂O₅), niobium, tungsten (W), iron (Fe), ruthenium oxide, rhodium oxide, copper oxide, zinc oxide, zirconium oxide, silicon dioxide, titanium oxide, hafnium oxide, aluminum oxide, vanadium oxide, niobium oxide, tungsten oxide, manganese, and iron oxide.

The catalyst material coated on the coils may remove nitrogen oxides (NO_{X}), sulfur oxides (SO_{X}), and volatile organic compounds (VOC) substances contained in the air introduced from the first raw material supply unit 110.

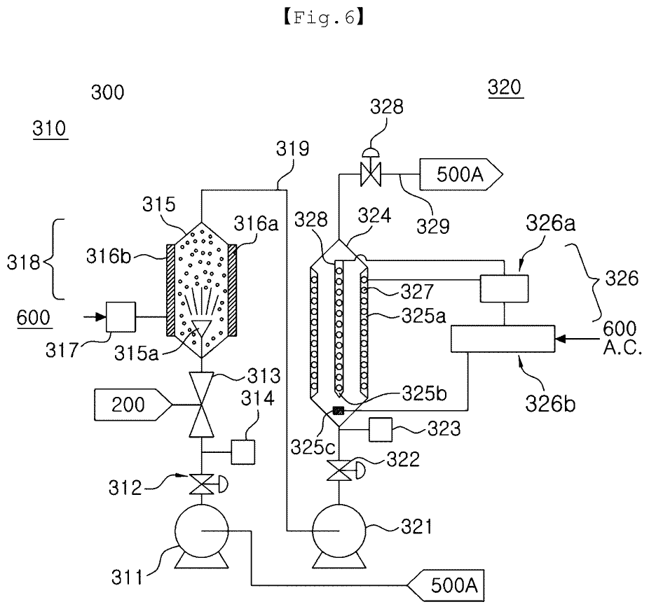

FIG. 6 is a cross-sectional view illustrating a dissolving device 300. Referring to the attached drawings, The dissolving device 300 includes a first dissolving device 310 and a second dissolving device 320.

The first dissolving device 310 includes a circulation pump 311, a flow rate control valve 312, a venturi injector 313, a pressure detection sensor 314, a main body (chamber) 315, a first quantum energy generator, defoamer 318 and a circulation line 319. The first quantum energy generator includes a first quantum energy generation coil 316a, a second quantum energy generation coil 316b and a pulse-type power supply unit 317.

The second dissolving device 320 includes a circulation pump 321, a flow rate control valve 322, pressure detection sensors 323 and 328, a chamber 324, a second quantum energy generator, and a circulation line 329. The second quantum energy generator includes a first quantum energy generation coil 325a, a second quantum energy generation coil 325b and a pulse-type power supply unit 326.

When power is supplied from the control panel 600 to the first circulation pump 311 in the above first dissolving device 310, the first circulation pump 311 is operated to suction and pressurize the aqueous solution of the main body 501 of the quantum energy transfer device 500 through the circulation line 319, and is adjusted to an appropriate flow rate in the flow control valve 312, and then supplied to the venturi ejector 313.

At the same time, quantum energy in the form of braking radiation in the ion generator 200 may be irradiated (200A and 200B), or a high voltage discharge process (200° C.) may be performed when quantum energy is irradiated. As a result, the covalent bonds of nitrogen molecules (N₂), oxygen molecules (O₂), water molecules (H₂O) of water vapor, hydrogen molecules (H₂), carbon dioxide (CO₂), and argon gases (Ar) are dissociated, and the dissociated nitrogen atoms (N), oxygen atoms (O), hydroxyl ions (OH⁻), hydrogen protons (H⁺), hydrogen ions (H⁻), carbon atoms (C), and nitric oxide (NO) may be supplied to the gas flow meter (not shown) installed on the supply pipe by pressing and self-pressure of the supply FANS 112 and 137 of the raw material supply unit 100, and after adjusting the appropriate flow rate, they may be supplied to the neck portion 313a of the venturi ejector 313.

Then, the dissociated gases are mixed with the process solution flowing into the venturi ejector 313 and introduced into the metal or ceramic disperser 313a with a cylindrical pore of less than 1 mm with a certain diameter and length installed inside the main body 210 in a fluid state in which gas and liquid are mixed, and then sprayed into the main body 315 as the bubble diameter is adjusted to less than 1 mm.

At the same time, a high voltage in the form of a pulse generated by the pulsed power supply unit 317 of the first quantum energy generator is attached to the outer surface of the body through a wire and supplied to Uniform saddle coil-shaped first and second quantum energy generating coils 316a and 316b in which the coil's winding directions are installed in opposite directions. The opposite pulse-shaped magnetic fields and electromotive forces act on the fluid flowing inside the body at a 90 degree angle in the current flow direction, and the opposite pulse-shaped magnetic fields overlap and offset (dissipate) in the center of the body, and quantum energy is generated in a zero magnetic field state and irradiated to the fluid inside the main body 315, defoaming (destroying) the bubble contained in the fluid, and first dissolving the gas introduced into the process solution and supplying it to the second dissolving device 320 by the suction force of the second pump 321.

The second dissolving device 320 includes a second circulation pump 321, a flow rate control valve 322, a pressure detection sensor 323, a chamber 324, a second quantum energy generator 327, a flow rate control valve 328, and a circulation line 329. The second quantum energy generator includes a first quantum energy generation coil 325a, a second quantum energy generation coil 325b, a third quantum energy generation coil 325c and a pulse-type power supply unit 326.

When the control panel (not shown) supplies power to the second circulation pump 321, the second circulation pump 321 is operated so that the process fluid in which the ionic gas is primarily dissolved in the first dissolving device 310 is sucked and pressurized by the second circulation pump 321 and introduced into the chamber (bubble breaking device) 324 to which the second quantum energy is irradiated through the flow rate adjusting valve 322.

The chamber 324 of the second dissolving device 320 to which the second quantum energy is irradiated has a cylindrical shape having a predetermined diameter and a predetermined length, and is insulated from the inner surface of the cylindrical chamber 324 so that a first quantum energy generation coil 325a having Solenoid coil shape is installed to be in surface contact with the inner surface. A second quantum energy generation coil 325b having Trigger coil shape is installed at intervals, and a third quantum energy generation coil 325c having a modified RF coil shape is installed downward at intervals from the first and second quantum energy generation coils 325a and 325b.

Trigger voltage generator 326b is installed at one side of the outside of the main body, is connected to the pulsed power supply unit 326 with a conducting wire.

The first and second quantum energy generating coils 325a and 325b are connected to a pulsed power supply unit 326a through a conducting wire, and the third quantum energy generating coil 325c is connected to the Trigger voltage generator 326b through the conducting wire. The pulsed power supply unit 326a and the Trigger voltage generator 326b are also connected through a conducting wire.

When the high voltage pulsed power generated by the pulsed power supply unit 326a is applied to the first and second energy generation coils 325a and 325b, a conducting wire may be sandwiched therebetween to apply a synchronous operation signal to the Trigger voltage generator 326b (or vice versa). Then, the Trigger voltage generator 326b applies the Trigger power generated by the Trigger voltage generator 326b to the third quantum energy generation coil 325c before (or simultaneously with) the high voltage pulse power is applied to the first and second quantum energy generation coils 325a and 325b by the high voltage pulsed power supply unit 326a.

Then, first, the third quantum energy generation coil 325c starts discharging while pulse energy is transferred between the first and second quantum energy generation coils 325a and 325b having polarities different from those of the magnetic field. The third quantum energy generation coil 325c starts main discharge at a lower voltage than the high voltage pulse applied to the first quantum energy generation coil 325a and the second quantum energy generation coil 325b.

That is, the discharge distance when the third quantum energy generation coil 325c is used is extended several times more than when the high voltage pulsed power supply unit 326a of the second quantum energy foaming device 327 applies the high voltage pulse to the first quantum energy generation coil 325a and the second quantum energy generation coil 325b to discharge the same voltage.

In addition, when a high voltage pulsed power supply unit 326a generates a pulse-type high voltage to the first quantum energy generation coil 325a and the second quantum energy generation coil 325b installed such that winding directions of the coils are opposite to each other through a conducting wire, a pulse-type magnetic field in opposite directions is generated in the first quantum energy generation coil 325a and the second quantum energy generation coil 325b at an angle of 90° with respect to a flow direction of a current, pulse-type magnetic fields in opposite directions are overlapped and offset (dissipated) at a center distance of the first quantum energy generation coil 325a and the second quantum energy generation coil 325b, and quantum energy is generated in a zero magnetic field state and irradiated to the introduced fluid, thereby improving discharge efficiency and improving a dissolution rate.

In addition, as pulse energy is transmitted to the fluid in which the gas and the liquid are mixed, a critical voltage of 1 volt or more is applied to the cell membrane of the bacteria in water, and thus the cell membrane of the bacteria is perforated in an electroporation method and discharge is started at the same time. A shock wave is generated by the discharge, and at the same time, bubbles (bubbles) containing ion gas, which are not destroyed in the first dissolving device 310 passing through between the first quantum energy generation coil 325a, the second quantum energy generation coil 325b, and the third quantum energy generation coil 325c, are destroyed by the discharge shock wave, and the water hammer pressure generated by the destruction of the bubbles efficiently further destroys the bubbles adjacent to the discharge shock wave.

The output end voltage of the high voltage pulsed power supply unit 326a is 1 kV to 30 kV range, the optimal voltage is selected and it selects. And the current value is 5 A to 300 A range, the optimal current value is selected and it selects. The pulse repetition rate (the pulse frequency per the unit time) selects an appropriate pulse repetition rate within a range of 20 Hz to 100 kHz, and the pulse width is within a range of 1 ms to 5 ms to supply the selected and output power to the first quantum energy generation coil 325a and the second quantum energy generation coil 325b through a conducting wire. The control unit (not shown) adjusts the output voltage of the high voltage pulsed power supply unit 326a based on the measurement data transmitted to the real-time control unit (not shown) by the bubble detection sensor (not shown).

If the bubble destruction rate is under the set target value, the pulse voltage or the pulse repetition rate is up-regulated and if the bubble destruction rate is over the set target value, the pulse voltage or the pulse repetition rate is down-regulated.

Materials of the first quantum energy generation coil 325a, the second quantum energy generation coil 325b, and the third quantum energy generation coil 325c may be selected any or more of stainless steel (STS304), titanium, one hearthalloy, iron, copper, aluminum, tin, and the like.

In the high voltage pulsed power supply unit 326a, an output voltage, an output current, a pulse repetition rate, and an output power having a preset pulse width are applied to the first quantum energy generation coil 325a and the second quantum energy generation coil 325b through a conducting wire, and a voltage generated by the Trigger voltage generator 326b is applied to the third quantum energy generation coil 325c through a conducting wire, so that pulse energy is transferred to the mixed fluid introduced between the first quantum energy generation coil 325a, the second quantum energy generation coil 325b, and the third quantum energy generation coil 325c, and a threshold voltage of 1 volt or more is applied to the cell membrane of the bacteria, so that the cell membrane of the bacteria is perforated in an electroporation method and discharge is started at the same time.

At the same time, bubbles that are not destroyed in the first dissolving device 310 passing between the first quantum energy generation coil 325a and the second quantum energy generation are destroyed by the discharge shock waves, and the water hammer pressure generated by the destruction of the bubbles efficiently further destroys the bubbles adjacent to the discharge shock waves.

However, depending on the size of the bubble, there may be a problem of reversely absorbing the discharge shock wave, but a water hammer pressure is generated at a bubble particle diameter of about 1.0 mm or less with a bubble particle diameter of about 1.0 mm as a boundary, and the discharge shock wave continues to act. On the contrary, bubbles having a large particle diameter of about 1.0 mm or more are destroyed and absorb the discharge shock wave.

Here, the bubble breaking water hammer pressure is a pressure generated in water when bubbles are broken.

In addition, the bubbles not only increase the discharge distance, but also destroy the bubbles by the discharge shock wave, and the water hammer pressure generated when the bubbles are destroyed is repeatedly increased by the discharge shock wave.

In the air bubbles, large and small air bubbles are mixed, but if the average particle diameter is 1 mm or less, the above-described “Repeated synergistic effect of water hammer pressure as discharge shock wave” and “the atmosphere of the air bubbles having the average particle diameter of 1 mm or less” result in raising effect of the water hammering pressure being repeated with the discharge shock wave.

In addition, during discharge, bubbles or plasma may be generated between the two electrodes. Plasma leaves ions or radicals as residues. In 20 Hz or greater, the high voltage pulse can be applied to the electrodes, therefore, these can be used for the discharge of the next time before the ion and radical disappear. In this case, ions and radicals increase the discharge distance more than the bubbles increase the discharge distance.

In addition, in destroying bubbles with the same particle diameter, as the discharge distance increases, ultra-high voltage and high current are required, and thus the size of the high voltage pulse generator 326 increases. In contrast, as the discharge distance decreases, the size of the high voltage pulse generator is also reduced, which can lead to lower voltage, lower cost, lower noise, and can safely and efficiently destroy bubbles.

In addition, even if the same discharge voltage is applied to the first quantum energy generation coil 325a and the second quantum energy generation coil 325b, the discharge distance increases as the number of microbubbles with small particle diameters in the water increases, thereby improving the bubble breaking ability.

Additionally, output power with output voltage, output current, pulse repetition rate, and pulse width set from the high voltage pulsed power supply unit 326a is applied to the first quantum energy generation coil 325a and the second quantum energy generation coil 325b through a conducting wire, and the voltage generated by the Trigger voltage generator 326b is applied to the third quantum energy generation coil 325c. As a result, the pulse energy is transferred between the first quantum energy generation coil 325a, the second quantum energy generation coil 325b, and the third quantum energy generation coil 325c, the additional voltage (V) is induced across the cell membrane of bacteria in the mixed fluid passing through between the first quantum energy generation coil 325a and the second quantum energy generation coil 325b, and the sum of the accumulated potential exceeds the threshold voltage between 200 mmV and 1V, transmembrane pores begin to form in the cell membrane. If the cell is exposed for a longer period of time with the potential of the cell membrane above the threshold, perforation causes the influx of extracellular ions, which leads to loss of homeostasis and subsequent apoptosis, resulting in irreversible cell death.

In the second dissolving device 320, the process liquid in which the ion gas is dissolved in the process liquid is white when large bubbles are destroyed and ultrafine bubbles are generated. When power generated by a control signal input to a control program in advance is supplied to the electric flow rate control valve in a control panel (not shown), the opening degree of the electric valve 328 is reduced and adjusted, and the drainage flow rate is adjusted. As a result, the pressure of the circulation line and the second dissolving device 320 installed on the discharge side of the second pump 321 is increased, and the ultrafine bubbles in the fluid are crushed (pressure-destroyed) to become a transparent process liquid.

In addition, the pressure adjustment of the circulation line and the second quantum energy foaming device 327 installed at the discharge side of the second pump 321 is measured in real time by the pressure detection sensor 323, and the control unit performs proportional adjustment based on data transmitted to the control unit (not shown).

The process liquid in which the ionic gas is dissolved in the dissolving device 300 is transferred to a first quantum energy transfer device 500A by the pressing force of the second circulation pump 321.

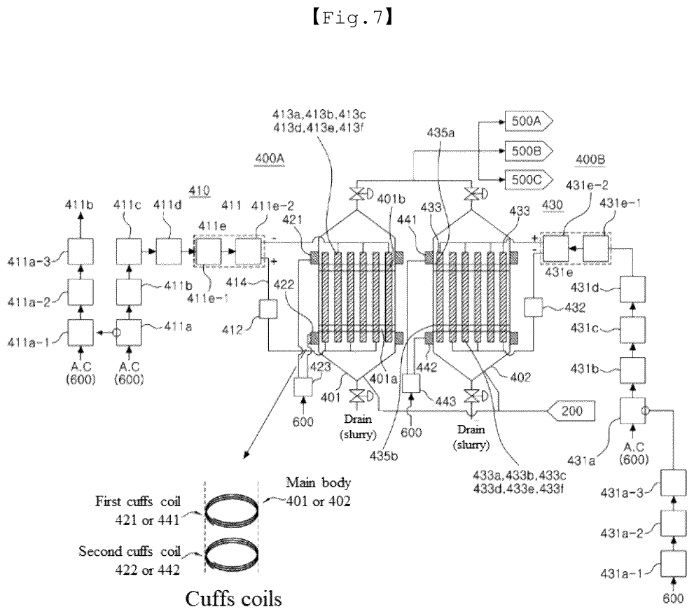

FIG. 7 is a schematic drawing illustrating the first and second ions separators 400A and 400B of FIG. 1. Referring to the attached drawings, the first ion separator 400A includes a positive charge collector 410 and a first quantum energy generaton device 420. The positive charge collector 410 includes a main body 401, a first power supply unit 411, a coil 412 and multiple positive charge collecting electrodes 410. And the first quantum energy generaton device 420 includes a first Cuffs coil 421, a second Cuffs coil 422 and a power supply unit 423.

The second ion separator 400B includes a negative charge collector 430 and a second quantum energy generaton device 440. The negative charge collector 430 includes a main body 402, a first power supply unit 431, a coil 432 and multiple negative charge collecting electrodes 433. And the second quantum energy generaton device 440 includes a first Cuffs coil 441, a second Cuffs coil 442 and a power supply unit 443.

The main body 401 of the first ion separator 400A may have a cylindrical shape with conical upper and lower parts, or a rectangular parallelepiped shape with rectangular pyramid shapes at upper and lower parts.

The plurality of first positive charge collecting electrodes 413 supplied with power generated by the power supply unit 411 of the positive charge collector 410 are installed in the upper and lower holders 401a and 401b at intervals from each other inside the main body 401 and at intervals from the upper, lower, left, and right inner surfaces of the main body 401.

The positive charge collector 410 includes the power supply unit 411, the first coil 412, the positive charge collecting electrodes 413, and a conducting wire 414.

In addition, the first Cuffs coil 421 of the first quantum energy generator 420 is installed in outer upper area of the main body 401, the second Cuffs coil 422 is installed at in outer lower area of the main body 401, and the power supply unit 423 is installed at an interval to supply the power generated from the power supply unit 423 to the first Cuffs coil 421 and the second Cuffs coil 422 through the conducting wire 424.

A material of the main body 401 may be selected any one of stainless steel (STS304, STS316L), glass fiber molding foam (FRP), steel (SS400), and hastelloy.

The positive charge collecting electrode 413 may be formed of any one of platinum (Pt), gold (Au), silver (Ag), copper (Cu) stainless steel (STS304, STS316L), and the like.

Pulse-type variable power is supplied to the first Cuffs coil 421 and the second Cuffs coil 422 wound in opposite directions through a wire from the power supply unit 423 of the first quantum energy generator 420. In the first Cuffs coil 421, a pulsed electromagnetic field (PEMF) is irradiated in the lower direction of the main body 401 at an angle of 90 degrees with the current flow direction, and in the second Cuffs coil 422, a pulsed electromagnetic field (PEMF) is irradiated in the upper direction of the main body 401 at an angle of 90 degrees with the current flow direction.

As a result, the pulse-type magnetic fields irradiated in opposite directions at the central distance between the first Cuffs coil 421 and the second Cuffs coil 422 overlap and offset (dissipate), and the pulsation quantum energy generated in the zero magnetic field state is irradiated into the central distance space between the first Cuffs coil 421 and the second Cuffs coil 422 inside the main body 401.

DC power in the form of a pulsed electromagnetic field generated by the power supply unit 411 of the first positive charge collector 410 is applied to the coils. The power supply unit 411 includes a variable voltage supply unit 411a, a rectification circuit 411b, an IGBT inverter 411c, a power output unit 411d, and a control unit 411e. The variable voltage supply unit 411a includes a control unit 411a-1 including a switching element in the form of an IGBT power supply unit, a first distribution resistor 411a-2 and a distribution circuit 411a-3. The control unit 411e includes a control signal generation unit 411e-1 and a microcomputer 411e-2.

The control unit 411a-1 of the variable voltage supply unit 411a includes N-th ports (Cpu P1, Cpu P2, Cpu PN) that selectively output high/low signals.

The first distribution resistor 411a-2-1 has first to N-th resistors (R1, R2, . . . , Rn) connected in series to each other at the side of the emitter, and a base terminal is connected to each of the first to Nth ports (Cpu P1, Cpu P2, . . . Cpu PN) of the control unit 411a-1, and is connected in parallel to the first to Nth transistors (not shown) connected in parallel.

The distribution circuit 411a-3 includes a second distribution resistor 411a-2-2 connected in series to the first to Nth transistors (not shown) and outputs a voltage divided between the first distribution resistor 411a-2-1 and the second distribution resistor 411a-2-2.

In the variable voltage supply 411a, the voltage of a single-phase AC power source of 220V and 60 Hz may be boosted in the range of a single-phase 1 kV to 30 kV or 30 kV to 1000 kV, or the voltage of a single-phase AC power source of 220V and 60 Hz may be reduced in the range of a single-phase 220V to 10V or 10V to 1V.

The rectifying circuit 411b may convert a single-phase 1 kV to 30 kV or 30 kV to 1000 kV to a DC power source, or a single-phase 220V to 10V or 10V to 1V to a DC power source.

The IGBT inverter 411c supplies DC power input by a control signal supplied from the outside, to the load side.

The control signal generation unit 411e-1 of the control unit 411e generates a control signal in the form of a pulse width modulation (PWM) control method, pulse frequency modulation (PFM), pulse frequency (density) modulation control (PDM), and pulse repetition rate (PRR) control and transmits it to the IGBT inverter 411c.

The voltage applied to the negative (−) terminal 413 and the pre-programmed input voltage are compared and adjusted, and the pre-programmed input voltage value is applied to the negative (−) terminal 413 through a conductive wire from the power output unit 411d.

The microcomputer 411e-2 of the control unit 411e converts the output signal of the control signal generation unit 411e-1 into a digital form to set the PWM width of the control signal from the received output signal, and transmits the control signal having the set width to the control signal generation unit 411e-1.

If a DC power source in the form of the pulsed electromagnetic field in range of a single-phase 1 kV to 30 kV, 30 kV to 1000 kV, 220V to 10V or 10V to 1V is supplied to multiple positive charge collecting electrodes through a wire, 90 in the current flow direction a magnetic field in the form of a pulsed electromagnetic field (PEMF) is generated at a degree angle.

In the positive charge collector 410, the output positive (+) terminal of the power supply unit 411 is directly connected to the first coil 412 through a conducting wire, but the output negative (−) terminal of the power supply unit 411 is connected to the plurality of first positive charge collecting electrodes 413a, 413c, and 413e through a conducting wire, and each of the second positive charge collecting electrodes 413b, 413d, and 413f is separated from the first positive charge collecting electrodes 413a, 413c, and 413e at an interval so that current does not flow.

The first coil 412 is directly supplied with positive (+) power of the power supply unit 411, and negative (−) power is not supplied to the first coil 412 installed to be connected to the second positive charge collecting electrodes 413b, 413d, and 413f installed separately from the first positive charge collecting electrodes 413a, 413c, and 413f connected to the output negative (−) terminal of the power supply unit 411. Therefore, between the first positive charge collecting electrodes 413a, 413c, and 413e and the second positive charge collecting electrodes 413b, 413d, and 413f, there is a strong attraction to supply negative (−) power.

As a result, hydrogen ions (H+, H) or oxygen ions (O+, O−) generated by dissociating hydrogen molecules or oxygen molecules in the first, second, and third ion generators 200A, 200B, and 200C are introduced into the first and second ion separators 400A and 400B and pass through between the first and second collection electrodes, and hydrogen ions (H+) or oxygen ions (O+) are adsorbed (captured) to the first charge collecting electrodes 413a, 413c, and 413e through which negative (−) currents flow and the second charge collecting electrodes 413b, 413d, and 413f in which attraction acts.

On the contrary, hydride ions (H−) or oxygen ions (O−) may be supplied to the energy transfer device 500 by the pressing force of the second circulation pump 321 by acting a repulsive force with the first charge collecting electrodes 413a, 413c, and 413e through which negative (−) current flows.

In another embodiment, when the first coil 412 is directly connected to the output-side negative (−) terminal of the power supply unit 411 of the charge collector 410 by a conducting wire, and the plurality of first charge collecting electrodes 413a, 413c, and 413e are connected to the output-side positive (+) terminal of the power supply unit 411 by a conducting wire, the respective second charge collecting electrodes 413b, 413d, and 413f are separated from the first positive charge collecting electrodes 413a, 413c, and 413e at intervals, so that current does not flow.

The first coil 412 is directly supplied with negative (−) power of the power supply unit 411, and positive (+) power is not supplied to the first coil 412 installed to be connected to the second charge collecting electrodes 413b, 413d, and 413f installed separately from the first charge collecting electrodes 413a, 413c, and 413f connected to the output positive (+) terminal of the power supply unit 411. Therefore, between the first charge collecting electrodes 413a, 413c, and 413e and the second charge collecting electrodes 413b, 413d, and 413f, there is a strong attraction to supply positive (+) power.

As a result, hydrogen ions (H⁺, H⁻) or oxygen ions (O⁺, O⁻) generated by dissociating hydrogen molecules or oxygen molecules in the first, second, and third ion generators 200A, 200B, and 200C are introduced into the first and second ion separators 400A and 400B and pass through between the first and second collection electrodes, and hydrogen ions (H⁻) or oxygen ions (O⁻) are adsorbed (captured) to the first charge collecting electrodes 413a, 413c, and 413e through which positive (+) currents flow and the second charge collecting electrodes 413b, 413d, and 413f in which attraction acts.

On the contrary, hydrogen ions (H⁺) or oxygen ions (O⁺) may be supplied to the energy transfer device 500 by the pressing force of the second circulation pump 321 by acting a repulsive force with the first charge collecting electrodes 413a, 413c, and 413e through which positive (+) current flows.

In addition, the field electron energy (eV) in which the covalent bond of the hydrogen molecule (H₂) is dissociated into the hydrogen atom (H⁺, H⁻) and the field electron energy (eV) in which the hydrogen atoms (H⁺, H⁻) are combined into the hydrogen molecule (H₂) are 15.4259 eV.

The field electron energy (eV) in which the covalent bond of the oxygen molecule (O₂) is dissociated into the oxygen atoms (O⁺, O⁻) and the field electron energy (eV) in which the oxygen atoms (O⁺, O⁻) are combined into the oxygen molecule (O₂) are 12.0697 eV.

Therefore, the power applied to the first coil 412 and the first positive charge collecting electrodes 413a, 413c, and 413e in the power supply 411 applies sufficient electric field electron energy that exceeds the covalent bond energy of the hydrogen molecule (H₂) and the oxygen molecule (O₂). In addition, the magnitude of attraction or repulsion acting between the first positive charge collecting electrodes (413a, 413c, and 413e) and the second positive charge collecting electrodes (413b, 413d, and 413f) installed at a certain distance apart from each other is applied with a value of field electron energy (eV) greater than the covalent bond of the hydrogen molecule (H₂) or the covalent bond of the oxygen molecule (O₂).

### SOURCE

1. (http://aceco.ca/wp-content/uploads/2015/03/Chemical-eV1.pdf)
2. chemical-covalent binding energy | creator flissiming

Furthermore, the first Cuffs coil 421 and the second Cuffs coil 422 are supplied with pulsed electromagnetic field (PEMF) power from the power supply unit 423 of the second quantum energy generator 420.

As a result, the pulsed electromagnetic field (PEMF) generated in the first Cuffs coil 421 and the second Cuffs coil 422 in opposite directions, is investigated in the interior space of the main body 401, overlapped and offset (dissipated), and pulsating quantum energy generated in the zero magnetic field state is investigated.

The irradiation of the pulsation quantum energy may extend a residence time of hydrogen ions (H⁺, H⁻) or oxygen ions (O⁺, O⁻) flowing into the first and second ion separators 400A and 400B from the first, second, and third ion generators 200A, 200B, and 200C, thereby improving separation efficiency of the cations (H⁺, O⁺).

The main body 402 of the second ion separator 400B may have a cylindrical shape with conical upper and lower parts, or a rectangular parallelepiped shape with rectangular pyramid shapes at upper and lower parts.

The plurality of first negative charge collecting electrodes 433 supplied with power generated by the power supply unit 431 of the negative charge collector 430 are installed in the upper and lower holders at intervals from each other inside the main body 402 and at intervals from the upper, lower, left, and right inner surfaces of the main body 402.

The negative charge collector 430 includes the power supply unit 431, the first coil 432, the negative charge collecting electrodes 433, and a conducting wire 434.

In addition, the first Cuffs coil 441 of the second quantum energy generator 440 is installed in the outer upper area of the main body 402, the second Cuffs coil 442 is installed in the outer lower area of the main body 402, and the power supply unit 443 is installed at an interval to supply the power generated from the power supply unit 443 to the first Cuffs coil 441 and the second Cuffs coil 442 through the conducting wire 444.

A material of the main body 402 may be selected any one of stainless steel (STS304, STS316L), glass fiber molding foam (FRP), steel (SS400), and hastelloy.

The negative charge collecting electrodes 433 may be formed of any one of platinum (Pt), gold (Au), silver (Ag), copper (Cu) stainless steel (STS304, STS316L), and the like.

Pulse-type variable power is supplied to the first Cuffs coil 441 and the second Cuffs coil 442 wound in opposite directions through a wire from the power supply unit 443 of the second quantum energy generator 440. In the first Cuffs coil 441, a pulsed electromagnetic field (PEMF) is irradiated in the lower direction of the main body 402 at an angle of 90 degrees with the current flow direction, and in the second Cuffs coil 442, a pulsed electromagnetic field (PEMF) is irradiated in the upper direction of the main body 402 at an angle of 90 degrees with the current flow direction.

As a result, the pulse-type magnetic fields irradiated in opposite directions at the central distance between the first Cuffs coil 441 and the second Cuffs coil 442 overlap and offset (dissipate), and the pulsation quantum energy generated in the zero magnetic field state is irradiated into the central distance space between the first Cuffs coil 441 and the second Cuffs coil 442 inside the main body 402.

DC power in the form of a pulsed electromagnetic field generated by the power supply unit 431 of the first negative charge collector 430 is applied to the coils. The power supply unit 431 includes a variable voltage supply unit 431a, a rectification circuit 431b, an IGBT inverter 431c, a power output unit 431d, and a control unit 431e. The variable voltage supply unit 431a includes a control unit 431a-1 including a switching element in the form of an IGBT power supply unit, a first distribution resistor 431a-2 and a distribution circuit 431a-3. The control unit 431e includes a control signal generation unit 431e-1 and a microcomputer 431e-2.

In the variable voltage supply 431a, the voltage of a single-phase AC power source of 220V and 60 Hz may be boosted in the range of a single-phase 1 kV to 30 kV, 30 kV to 1000 kV, or the voltage of a single-phase AC power source of 220V and 60 Hz may be reduced in the range of a single-phase 220V to 10V or 10V to 1V.

The rectifying circuit 431b may convert a single-phase 1 kV to 30 kV, 30 kV to 1000 kV to a DC power source, or a single-phase 220V to 10V or 10V to 1V to a DC power source.

The IGBT inverter 431c supplies DC power input by a control signal supplied from the outside, to the load side.

The control signal generation unit 431e-1 of the control unit 431e generates a control signal in the form of a pulse width modulation (PWM) control method, pulse frequency modulation (PFM), pulse frequency (density) modulation control (PDM), and pulse repetition rate (PRR) control and transmits it to the IGBT inverter 431c.

The voltage applied to the positive (+) terminal 433 and the pre-programmed input voltage are compared and adjusted, and the pre-programmed input voltage value is applied to the positive (+) terminal 433 through a conductive wire from the power output unit 431d.

The microcomputer 431e-2 of the control unit 431e converts the output signal of the control signal generation unit 431e-1 into a digital form to set the PWM width of the control signal from the received output signal, and transmits the control signal having the set PWM width to the control signal generation unit 431e-1.

If a DC power source in the form of the pulsed electromagnetic field in range of a single-phase 1 kV to 30 kV, 30 kV to 1000 kV, 220V to 10V or 10V to 1V is supplied to multiple negative charge collecting electrodes through a wire, 90 in the current flow direction a magnetic field in the form of a pulsed electromagnetic field (PEMF) is generated at a degree angle.

In the negative charge collector 430, the output negative (−) terminal of the power supply unit 431 is directly connected to the second coil 432 through a conducting wire, but the output positive (+) terminal of the power supply unit 431 is connected to the plurality of first charge collecting electrodes 433a, 433c, and 433e through a conducting wire, and each of the second charge collecting electrodes 433b, 433d, and 433f is separated from the first charge collecting electrodes 433a, 433c, and 433e at an interval so that current does not flow.

The second coil 432 is directly supplied with negative (−) power of the power supply unit 431, and positive (+) power is not supplied to the second coil 432 installed to be connected to the second charge collecting electrodes 433b, 433d, and 433f installed separately from the first charge collecting electrodes 433a, 433c, and 433f connected to the output positive (+) terminal of the power supply unit 431. Therefore, between the first charge collecting electrodes 433a, 433c, and 433e and the second charge collecting electrodes 433b, 433d, and 433f, there is a strong attraction to supply positive (+) power.

As a result, hydrogen ions (H⁺, H) or oxygen ions (O⁺, O⁻) generated by dissociating hydrogen molecules or oxygen molecules in the first, second, and third ion generators 200A, 200B, and 200C are introduced into the first and second ion separators 400A and 400B and pass through between the first and second collection electrodes, and hydrogen ions (H⁻) or oxygen ions (O⁻) are adsorbed (captured) to the first charge collecting electrodes 433a, 433c, and 433e through which positive (+) currents flow and the second charge collecting electrodes 433b, 433d, and 433f in which attraction acts.

On the contrary, hydrogen ions (H⁺) or oxygen ions (O⁺) may be supplied to the energy transfer device 500 by the pressing force of the second circulation pump 321 by acting a repulsive force with the first charge collecting electrodes 433a, 433c, and 433e through which positive (+) current flows.

In another embodiment, when the second coil 432 is directly connected to the output-side positive (+) terminal of the power supply unit 431 of the charge collector 430 by a conducting wire, and the plurality of first charge collecting electrodes 433a, 433c, and 433e are connected to the output-side negative (−) terminal of the power supply unit 431 by a conducting wire, the respective second charge collecting electrodes 433b, 433d, and 433f are separated from the first charge collecting electrodes 433a, and 433c, 433e at intervals, so that current does not flow.

The second coil 432 is directly supplied with positive (+) power of the power supply unit 431, and negative (−) power is not supplied to the second coil 432 installed to be connected to the second charge collecting electrodes 433b, 433d, and 433f installed separately from the first charge collecting electrodes 433a, 433c, and 433f connected to the output negative (−) terminal of the power supply unit 431. Therefore, between the first charge collecting electrodes 433a, 433c, and 433e and the second charge collecting electrodes 433b, 433d, and 433f, there is a strong attraction to supply negative (−) power.

As a result, hydrogen ions (H⁺, H⁻) or oxygen ions (O⁺, O⁻) generated by dissociating hydrogen molecules or oxygen molecules in the first, second, and third ion generators 200A, 200B, and 200C are introduced into the first and second ion separators 400A and 400B and pass through between the first and second collection electrodes, and hydrogen ions (H⁺) or oxygen ions (O⁺) are adsorbed (captured) to the first charge collecting electrodes 433a, 433c, and 433e through which negative (−) currents flow and the second charge collecting electrodes 433b, 433d, and 433f in which attraction acts. On the contrary, hydride ions (H⁻) or oxygen ions (O⁻) may be supplied to the energy transfer device 500 by the pressing force of the air FAN 137 by acting a repulsive force with the first charge collecting electrodes 433a, 433c, and 433e through which negative (−) current flows.

In addition, the field electron energy (eV) in which the covalent bond of the hydrogen molecule (H₂) is dissociated into the hydrogen atom (H⁺, H⁻) and the field electron energy (eV) in which the hydrogen atoms (H⁺, H⁻) are combined into the hydrogen molecule (H₂) are 15.4259 eV.

The field electron energy (eV) in which the covalent bond of the oxygen molecule (O₂) is dissociated into the oxygen atoms (O⁺, O⁻) and the field electron energy (eV) in which the oxygen atoms (O⁺, O⁻) are combined into the oxygen molecule (O₂) are 12.0697 eV.

Therefore, the power applied to the first coil 432 and the first negative charge collecting electrodes 433a, 433c, and 433e in the power supply unit 431 applies sufficient electric field electron energy that exceeds the covalent bond energy of the hydrogen molecule (H₂) and the oxygen molecule (O₂). In addition, the magnitude of attraction or repulsion acting between the first negative charge collecting electrodes (413a, 413c, and 413e) and the second negative charge collecting electrodes (413b, 413d, and 413f) installed at a certain distance apart from each other is applied with a value of field electron energy (eV) greater than the covalent bond of the hydrogen molecule (H₂) or the covalent bond of the oxygen molecule (O₂).

### SOURCE

1. (http://aceco.ca/wp-content/uploads/2015/03/Chemical-eV1.pdf)
2. chemical-covalent binding energy | creator flissiming

Furthermore, the first Cuffs coil 441 and the second Cuffs coil 442 are supplied with pulsed electromagnetic field (PEMF) power from the power supply unit 443 of the second quantum energy generator 440.

As a result, the pulsed electromagnetic field (PEMF) generated in the first Cuffs coil 441 and the second Cuffs coil 442 in opposite directions, is investigated in the interior space of the main body 402, overlapped and offset (dissipated), and pulsating quantum energy generated in the zero magnetic field state is investigated.

The irradiation of the pulsation quantum energy may extend a residence time of hydrogen ions (H⁺, H⁻) or oxygen ions (O⁺, O⁻) flowing into the second ion separator 400B, thereby improving separation efficiency of the negative charge ions (H⁻, O⁻).

FIG. 8 is a schematic drawing illustrating the third ion separator 400C of FIG. 1. Referring to the accompanying drawings, the third ion separator 400C includes a reactor 440, a condenser 450 and a positive charge collector 457.

The reactor 440 includes a main body 403, a jacket 403a, a cooling water circulation line 444a, a hot water circulation line 444b, a municipal water or purified water supply line 445, a raw material input line 446, and a stirrer 447. The condenser 450 includes a main body 451, a cooling water circulation line 451a, and a exhaust line 452 of hydrogen ions (H⁻) or oxygen ions (O⁻). The positive charge collector 457 includes a plurality of first cathodes (453a, 453b, 453c, 453d), the first coil (454; the load), second power supply unit 455, and conducting wire 456.

The lower part of the main body 403 of the reactor 440 is conical, the upper part of the main body 403 is inclined, and the main body 403 may have a dual-structure cylindrical shape including a jacket 403a.

The cooling water supply and exhaust line 444a, and the hot water supply and exhaust line 444b may be connected and installed on one side of the side lower portion and one side of the side upper portion of the jacket 403a.

The municipal water or the purified water supply line 445 may be installed on the upper inclined part of the main body 403.

A stirrer 447 is installed at the center of the upper end surface of the main body 403. A pipe connected to the input hopper 446 of alkali metal material that generates hydride ions (H−) is installed on one side of the upper left side of the body 403. On one side of the upper right side of the body 403, a supply pipe 452 that discharges hydride ions (H−) generated in the reactor 440 and supplies them to the condenser 450 is installed and connected to the center of the right-axis cell 451a of the condenser body 451.

An anode 441 and a cathode 442 for electrolysis having a predetermined area are installed to face each other on an inner side surface of the reactor main body 403, a first power supply unit 443 for supplying DC power in a pulse form to the anode 441 and the cathode 442 by a conducting wire is installed on an outer side surface of the reactor main body 403.

The shape of the condenser 450 may be a shell and tube type.

Hydrogen ion (H⁻) supply line 452 of the reactor 440 is installed, connected to the center of a shell on the right side of the main body 451 of the condenser 450. The hydrogen ions (H⁻) or the oxygen ions (O⁻) supply line 452 is connected and installed in the center of the shell on the left side of the main body 451 of the condenser 450 to supply hydrogen ions (H⁻) or oxygen ions (O⁻) removed by condensing moisture by the cooling function of the condenser 450 to the first, second, and third quantum energy transfer devices (500A, 500B, 500C).

A cooling water supply line 451a is installed on one side surface of the right lower portion of the main body 451 of the condenser 450, and a cooling water exhaust line 451a is installed on one side surface of the upper portion of the left side surface of the main body 451.

First cathodes (453a-1, 453b-1, 453c-1, and 453d-1) and second cathodes (453a-2, 453b-2, 453c-2 and 453d-2) of the positive charge collectors 457 are installed to face each other in each of the tubes 453a, 453b, 453c, and 453d inside the main body 451, respectively. And a first coil 454 and a second power supply unit 455 of the positive charge collector 457 are installed at one side of the outside of the main body 451.

The output side negative (−) terminal of the second power supply unit 455 of the positive charge collector 457 is connected to each of the plurality of first negative electrodes 453a-1, 453b-1, 453c-1, and 453d-1, the output side positive (+) terminal of the second power supply unit 455 is connected to the first coil 454, and another conducting wire of the first coil 454 is connected to the second negative electrodes 453a-2, 453b-2, 453c-2, and 453d-2 spaced apart from the first negative electrodes 453a-1, 453b-1, 453c-1, and 453d-1.

Materials of the reactor body 403 and the condenser 450 may be any one of stainless steel (STS304, STS316L), glass fiber molding foam (FRP), steel (SS400), and hastelloy.

The first negative electrodes 453a-1, 453b-1, 453c-1, and 453d-1 and the second negative electrodes 453a-2, 453b-2, 453c-2, and 453d-2 of the positive charge collector 457 may made of any one of platinum (Pt), gold (Au), silver (Ag), copper (Cu) stainless steel (STS304, STS316L), and the like.

The cooling water supply and exhaust line 444a, and a hydrogen ions (H⁻) supply and exhaust line 452 may be any one material of the stainless steel (STS304, STS316L), the copper pipe (cu), and the zinc steel pipe.

### Generation of Hydride Ions (H⁻)

1. The electronic valve 445a of the municipal water or the purified water supply line 445 is opened and the municipal water or the purified water is filled with 60 to 75% of the volume of the main body 403 of the container 440.
2. The electronic valve of the hot water supply and exhaust line 444a is opened, and the hot water are circulated from the lower supply line of the jacket 403a to the top exhaust line 444a and heat the municipal water or the purified water filled with appropriate amount inside the main body 403 to 50 to 65 degrees Celsius.
3. The mixer 447 is operated, and the mixer 447 stirs the municipal water or the purified water in the range of 30 to 60 RPM.
4. The pulse-type DC power generated by the power supply unit 443 is supplied to the first electrode 441 and the second electrode 442 installed on the inner side surface of the reactor 440.
5. The cover of the hopper 446 is opened, and the metal hydrate in which one kind material is selected from the lithium hydride (LiH), the sodium hydride (NaH) and the potassium hydride (KH) is supplied to the gravity difference to the main body 403.

Hydride ions (H⁻) may be generated by a hydrolysis reaction and an electrolysis reaction between the metal hydrate and the water.

As the pulse-type DC power generated by the first power supply unit 443 is supplied to the first electrode 441 and the second electrode 442, the metal hydrate introduced into the main body 403 is subjected to an electrolysis reaction to generate a Hydrogen ions (H⁻) as shown in formula 1, formula 2 and formula 3.

1) The reaction of LiH

LiH→Li⁺+H⁻ formula 1

2) The reaction of NaH

NaH→Na⁺+H⁻ formula 2

3) reaction of KH

KH→K⁺+H⁻ formula 3

Hydride ions (H−) generated by the hydration reaction of Formulas 1, 2, and 3 flow into the main body 451 of the condenser 450 through the supply line 452 and are cooled by indirect heat exchange with cooling water introduced into the cooling water supply line and the exhaust line 451 installed on one side of the lower and upper sides of the body 451 to condense and liquefy the water vapor of water molecules mixed with hydrogen ions (H⁻).

The liquefied water is discharged to the outside through a condensate exhaust line (not shown) installed under the left cell 451a of the main body 451 so that hydride ions (H⁻) and water vapor are separated and introduced into the first, second, and third quantum energy transfer devices (500A, 500B, and 500C).

In this process, positive charges may be collected on the first and second cathodes to suppress a reaction between hydride ions (H⁻) and a small amount of lithium cation (Li⁺) or sodium cation (Na⁺) and potassium cation (K⁺).

Specifically, negative (−) terminals on the output side of the power supply unit 455 of the positive charge collector 457 are connected to each of the plurality of first negative electrodes 453a-1, 453b-1, 453c-1, and 453d-1 of the positive charge collector, positive (+) terminals are connected to the first coil 454, and another electric wire of the first coil 454 is connected to the second negative electrodes 453a-2, 453b-2, 453c-2, and 453c-2. The second negative electrodes 453a-2, 453b-2, 453c-2, and 453c-2 are installed to be separated from the first negative electrodes 453a-1, 453b-1, 453c-1, and 453d-1.

Negative (−) power is supplied to the first negative electrodes 453a-1, 453b-1, 453c-1, and 453d-1, and no power is supplied to the second negative electrodes 453a-2, 453b-2, 453c-2, and 453d-2, therefore, between the first negative electrodes 453a-1, 453b-1, 453c-1, and 453d-1 and the second negative electrodes 453a-2, 453b-2, 453c-2, and 453d-2, there is a strong attraction to supply negative (−) power.

Therefore, lithium ions (Li⁺), sodium ions (Na⁺), and potassium ions (K⁺) are collected in the first and second cathodes inside the condenser, and hydrogen ions (H⁻) are separated from the cations by repulsive force and introduced into the first, second, and third quantum energy transfer devices (500A, 500B, 500c) by the pressing force of the air FAN (not shown).

In addition, the field electron energy (eV) in which the covalent bond of the hydrogen molecule (H₂) is dissociated into the hydrogen atom (H⁺, H⁻) and the field electron energy (eV) in which the hydrogen atoms (H⁺, H⁻) are combined into the hydrogen molecule (H₂) are 15.4259 eV.

The field electron energy (eV) in which the covalent bond of the oxygen molecule (O₂) is dissociated into the oxygen atoms (O⁺, O⁻) and the field electron energy (eV) in which the oxygen atoms (O⁺, O⁻) are combined into the oxygen molecule (O₂) are 12.0697 eV.

Therefore, the power applied to the first coil 454 and the first cathodes (453a-1, 453b-1, 453c-1, and 453d-1) in the power supply unit 455 applies sufficient electric field electron energy that exceeds the covalent bond energy of the hydrogen molecule (H₂) and the oxygen molecule (O₂). In addition, the magnitude of attraction or repulsion acting between the first cathodes (453a-1, 453b-1, 453c-1, and 453d-1) and the second cathodes (453a-2, 453b-2, 453c-2, 453d-2) installed at a certain distance apart from each other is applied with a value of field electron energy (eV) greater than the covalent bond of the hydrogen molecule (H₂) or the covalent bond of the oxygen molecule (O₂).

### SOURCE

1. (http://aceco.ca/wp-content/uploads/2015/03/Chemical-eV1.pdf)
2. chemical-covalent binding energy | creator flissiming

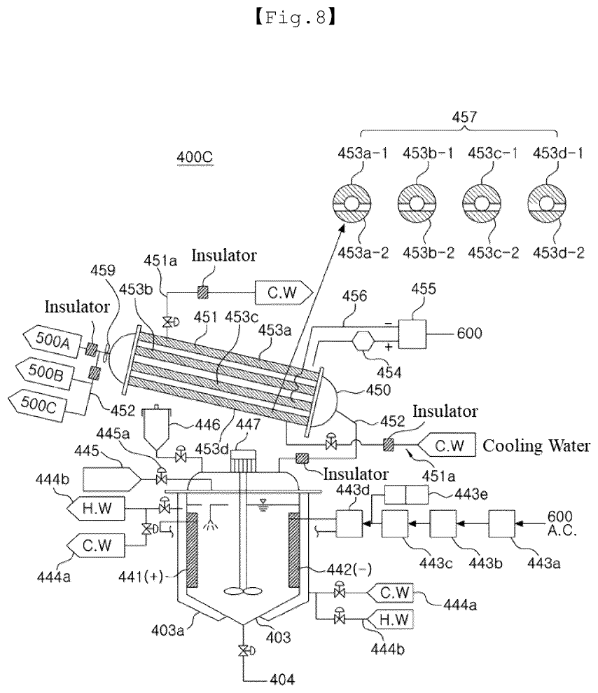

FIG. 9 is a schematic drawing illustrating the first quantum energy transfer device 500A of FIG. 1.

Referring to the accompanying drawings, the first quantum energy transfer device 500A includes an ion gas supply line 501a, a municipal water or purified water supply line 501b, a solution supply line 501c in which ions are dissolved, a circulation and exhaust line 505a and 505b, a first main body 501, a second main body 502, a stirrer 503, a circulation pump 504, a first quantum energy generation device 510, and a second quantum energy generation device 520. The first quantum energy generation device 510 includes a first quantum energy generation coil 511, a second quantum energy generation coil 512, a first power supply unit 513 for supplying power in a pulse form, and a conducting wire 514. The second quantum energy generation device 520 includes a third quantum energy generation coil 521, a fourth quantum energy generation coil 522, a second power supply unit 523 for supplying power in a pulse form, and a conducting wire 524.

A solution containing hydrogen ions (H⁺, H⁻) or oxygen ions (O⁺, O⁻) in the dissolving device 300 is sent to the supply line 501c by the pressing force of the pressure pump 321, followed by 70-80% of the internal volume of the main body 501 through the venturi ejector 506.

Alternatively, a first solution in which hydrogen ions (H⁻) or oxygen ions (O⁻)) supplied from the first and third ion separators (400A, 400C) or hydrogen ions (H⁺) or oxygen ions (O⁺) supplied from the secondary ion separator 400B are mixed with purified water (municipal water) may be filled to 70 to 80% of the internal volume of the main body 501 through the supply line (501B) and the venturi ejector 506.

Alternatively, a second solution in which hydrogen ions (H⁻) or oxygen ions (O⁻) supplied from the first and third ion separators (400A, 400C) or hydrogen ions (H⁻) or oxygen ions (O⁻) supplied from the secondary ion separator 400B are mixed with purified water (municipal water) may be filled to 70 to 80% of the internal volume of the main body 501 through the supply line 501B and the venturi ejector 506.

In this process, the level sensor (not shown) detects the water level and transmits it to the control panel 600, and the control panel 600 stops the pressure pump 321 and supplies power to the stirrer 501 to rotate the first solution or the second solution supplied to the main body 501 clockwise at 30 to 90 RPM. Alternatively, in the case of the solution introduced from the second ion separator 400B, the solution may be rotated counterclockwise at 30 to 90 RPM.

At the same time, when pulsed direct current power is supplied from the first power supply 513 to the first and second quantum energy generating coils 511 and 512 in opposite directions through a wire, an electromagnetic field in the form of a pulse is generated at an angle of 90 degrees in the current flow direction and transferred to the first solution or the first solution. The inside of the main body 501 has a rectangular flat plate shape of an insulating material installed to face each other, and the first and second quantum energy generating coils 511 and 512 may have any one of Helmhelz coil, Cuffs coil, Trigger coil, and Solenoid coil transformed into a cone shape at intervals from each other on a flat plate.

As a result, electromagnetic fields in the form of pulses in opposite directions overlap and offset (dissipate) in the center of the first body 501, generating quantum energy in a zero magnetic field state, and the quantum energy above can be irradiated to the solution.

Subsequently, when power is supplied to the circulation pump 504 from the control panel 600, the solution irradiated with electromagnetic fields and quantum energy is sucked and pressurized inside the first body 501 and supplied to the second body 502.

The plurality of third quantum energy generation coils 521 and the plurality of fourth quantum energy generation coils 522 have a disk shape as a whole, and may have any one of a modified Helmheltz coil, Cuffs coil, Trigger coil, and Solenoid coil shape.

The third quantum energy generating coils 521 and the fourth quantum energy generating coils 522 may be wound in opposite directions, and an inlet and an outlet hole may be perforated between the coil and the coil wound with a predetermined winding number.

The pulse-type power generated by the second power supply unit 513 is applied to the third quantum energy generating coils 521, in which the coils are wound in opposite directions, and the pulse-type electromagnetic field generated at an angle of 90 degrees in the current flow direction is installed to face each other. In addition, the opposite pulse-type electromagnetic energy generated between the third quantum energy generating coils 521 and the fourth quantum energy generating coils 522 may be irradiated to the solution passing through the inlet and outlet holes. The electromagnetic energy in the form of pulses in opposite directions overlap and offset (dissipate), and the pulse-type quantum energy generated in the zero magnetic field state is irradiated and continues to circulate for a certain period of time, before the electric valve 505a installed on the circulation line 505 above the second body 502 is closed and the electric valve 505b is opened to supply the solution to which the electromagnetic field and quantum energy are irradiated.

Alternatively, the electric valves 505b and 505d installed on the circulation pipe 505 are closed, and the electric valve 505c is opened, so that a solution irradiated with electromagnetic fields and quantum energy can be supplied to the first ion dissolving device 310.

The first power supply unit 513 applying the pulse power to the first and second quantum energy generation coils 511 and 512 includes a pressure reduction (pressure increase) transformer 513a, a rectifier circuit 513b, and a control unit 513c, the control unit 513c includes an input module 513c-1, a calculation module 513c-2, and a control module 513c-3 including a pulse width modulation (PWM) control method, a pulse frequency modulation (PFM), a pulse frequency (density) control (PDM), and a pulse repetition rate control (PRR) function.

When AC power of 220 V and 60 Hz is supplied to the pressure reduction (pressure reduction) transformer 513a, the AC power may be increased from the pressure reduction transformer 513a to AC power of 1-10 kV and 60 Hz or decreased from the pressure reduction transformer 513a to AC power of 1-100 V and 60 Hz.

The input module 513c-1 of the control unit 513c inputs, to a program embedded therein, variables such as a current value, a voltage value, a pulse width, a pulse density pulse period, a frequency burst length, a main power supply time and a stop time (timer function), a switching element function, and a surface temperature control function of the quantum energy generation coils 511 and 512, which are supplied to the first and second magnetic field generation coils 511 and 512, i.e.,
a minimum value mA and a maximum value A of the variable current flowing through the first and second magnetic field generation coils 511 and 512, the minimum value (mT) and the maximum value (T) of the variable magnetic field, the power supply time (seconds to minutes, or minutes to hours) for each step, and the like. Each of the parameters may be set on the monitor (not shown) of the input module 513c-1, for example, the operation step among the steps divided into steps 1 to 10, and then data of the selected step may be input.

The calculation module 513c-1 may calculate the amount of current corresponding to the intensity of the magnetic field to be generated in the first magnetic field generation coil 511 and the second magnetic field generation coil 512 for generating variable quantum energy by executing a calculation program on a plurality of parameters input by the user to the input module 513c-1, and may generate a current value by generating corresponding data in advance and matching the input frequency band, the magnetic field intensity value, and the current value one-to-one, measure the magnetic field of the first quantum energy generation coil 511 of the first quantum energy generator by a magnetic field sensor (not shown), calculate a resonance frequency value capable of making a zero magnetic field state by overlapping the magnetic field generated in the first and second quantum energy generation coils 511 and 512 of the first quantum energy generator 510 with the data transmitted in real time, and generate a current value proportional to the calculated resonance frequency value. For example, when the intensity value of the magnetic field is between 1 and 10, data about a current value corresponding to between 1 and 10 is generated, and when the intensity value of the magnetic field is 1, a power of a pulsed electromagnetic field (PEMF) type having a current value corresponding to 1 is generated. In addition, the magnetic field sensor 513e measures the magnetic fields of the first and second quantum energy generation coils 511, 512 of the first quantum energy generator 510, and corrects the intensity value and current value of the magnetic field according to the data transmitted in real time.

The current control module 513c-1 generates a current corresponding to the strength of the magnetic field to be generated in the first magnetic field generating coil 511 and the second magnetic field generating coil 512 according to the current value transmitted from the calculation module 513c-2, supplies the current to the first quantum energy generating coil 511 and the second quantum energy generating coil 512, detects a current value flowing in one or each of the lead-in lines of the first and second quantum energy generating coils 511 and 512 by a current detection sensor 513d installed in one or each of the lead-in lines of the first and second quantum energy generating coils 511 and 512, and transmits the detected current value to the calculation module 513c-2 in real time. Then, the current control module 513c-1 compares the current value with a set value for each parameter input by a user, executes a calculation program when the current value deviates from the upper and lower limit values of the set value, and transmits a set value for each modified parameter so that they can be restored to the settings.

The current control module 513c-3 supplies the power of the pulse type (PEMF) suitable for the current modified for each step variable to the first quantum energy generation coil 511 and the second quantum energy generation coil 512, or the power of the pulse type (PEMF) suitable for the magnetic field value corresponding to the resonance frequency and the corrected current value corresponding to the resonance frequency by data transmitted by the magnetic field detection sensor 513e is supplied to the first quantum energy generation coil 511 and the second quantum energy generation coil 512 or the pulse type (PEMF) power is supplied to each of the first and second quantum energy generation coils.

In addition, the calculation module 513c-2 compares the generated current value with the received current value digitized from the first quantum energy generation coil 511 and the second quantum energy generation coil 512 received from the current detection sensor 513d, and transmits a current value required through PID control according to the difference to the current control module 513c-3.

The magnetic field detection sensor 513e may be any one of a super conducting quantum interference device (SQUID) sensor, a nuclear magnetic resonance (NMR) sensor, an atomic magnetic resonance (AMR) sensor, a flux gate sensor, a magnetic resistance (MR) sensor, a magnetic impedance (MI) sensor, a hall effect sensor, an optical fiber magnetic sensor, and a search coil.

In the first power supply unit 513, the frequency modulation range may be in the range of 1 Hz to 100 Hz, 100 Hz to 1 kHz, 1 kHz to 10 kHz, 10 kHz to 1 MHz, 1 MHz to 100 MHz, and 100 MHz to 10 GHZ.

The second power supply unit 523 configured to apply the pulse power to the third quantum energy generation coils 511 and 512 includes a pressure-reduced transformer 523a, a rectifier circuit 523b, and a control unit 523c, the control unit 523c includes an input module 523c-1, a calculation module 523c-2, and a control module 523c-3 including a pulse width modulation (PWM) control method, a pulse frequency modulation PFM (pulse frequency modulation), a pulse frequency (density) control (PDM), and a pulse repetition rate control (PRR) function. When AC power of 220 V and 60 Hz is supplied to the pressure-increasing transformer 523a, the AC power is increased from the pressure-increasing transformer 523a to AC power of 1 to 10 kV and 60 Hz or reduced to AC power of 1 to 100 V and 60 Hz and supplied to the rectifier 523b, the AC power is converted from the rectifier 523b to DC power of 1 to 100 V or 1 to 10 kV.

The input module 523c-1 of the control unit 523c inputs, to a program embedded therein, variables such as a current value, a voltage value, a pulse width, a pulse density pulse period, a frequency burst length, a main power supply time and a stop time (timer function), a switching element function, and a surface temperature control function of the quantum energy generation coils 521 and 522, which are supplied to the first and second magnetic field generation coils 521 and 522, i.e.,
a minimum value mA and a maximum value A of the variable current flowing through the first and second magnetic field generation coils 521 and 522, the minimum value (mT) and the maximum value (T) of the variable magnetic field, the power supply time (seconds to minutes, or minutes to hours) for each step, and the like. Each of the parameters may be set on the monitor (not shown) of the input module 523c-1, for example, the operation step among the steps divided into steps 1 to 10, and then data of the selected step may be input.

The calculation module 523c-1 may calculate the amount of current corresponding to the intensity of the magnetic field to be generated in the first magnetic field generation coil 521 and the second magnetic field generation coil 522 for generating variable quantum energy by executing a calculation program on a plurality of parameters input by the user to the input module 523c-1, and may generate a current value by generating corresponding data in advance and matching the input frequency band, the magnetic field intensity value, and the current value one-to-one, measure the magnetic field of the first quantum energy generation coil 521 of the first quantum energy generator by a magnetic field sensor (not shown), calculate a resonance frequency value capable of making a zero magnetic field state by overlapping the magnetic field generated in the first and second quantum energy generation coils 521 and 522 of the second quantum energy generator 520 with the data transmitted in real time, and generate a current value proportional to the calculated resonance frequency value. For example, when the intensity value of the magnetic field is between 1 and 10, data about a current value corresponding to between 1 and 10 is generated, and when the intensity value of the magnetic field is 1, a power of a pulsed electromagnetic field (PEMF) type having a current value corresponding to 1 is generated. In addition, the magnetic field sensor (not shown) measures the magnetic fields of the first and second quantum energy generation coils 521, 522 of the second quantum energy generator 520, and corrects the intensity value and current value of the magnetic field according to the data transmitted in real time.

The current control module 523c-1 generates a current corresponding to the strength of the magnetic field to be generated in the first magnetic field generating coil 521 and the second magnetic field generating coil 522 according to the current value transmitted from the calculation module 523c-2, supplies the current to the first quantum energy generating coil 521 and the second quantum energy generating coil 522, detects a current value flowing in one or each of the lead-in lines of the first and second quantum energy generating coils 521 and 522 by a current detection sensor (not shown) installed in one or each of the lead-in lines of the first and second quantum energy generating coils 521 and 522, and transmits the detected current value to the calculation module 523c-2 in real time. Then, the current control module 523c-1 compares the current value with a set value for each parameter input by a user, executes a calculation program when the current value deviates from the upper and lower limit values of the set value, and transmits a set value for each modified parameter so that they can be restored to the settings.

The current control module 523c-3 supplies the power of the pulse type (PEMF) suitable for the current modified for each step variable to the first quantum energy generation coil 521 and the second quantum energy generation coil 522, or the power of the pulse type (PEMF) suitable for the magnetic field value corresponding to the resonance frequency and the corrected current value corresponding to the resonance frequency by data transmitted by the magnetic field detection sensor (not shown) is supplied to the first quantum energy generation coil 521 and the second quantum energy generation coil 522 or the pulse type (PEMF) power is supplied to each of the first and second quantum energy generation coils.

In addition, the calculation module 523c-2 compares the generated current value with the received current value digitized from the first quantum energy generation coil 521 and the second quantum energy generation coil 522 received from the current detection sensor (not shown), and transmits a current value required through PID control according to the difference to the current control module 523c-3.

The items input to the input modules 513c-1 and 523c-1 of the control units 513c and 523c of the first power supply unit 513 and the second power supply unit 523 include the minimum (mA) and the maximum value (A) of the variable current of the power to be applied to the first quantum energy generation coil 511, the second quantum energy generation coil 512, the third quantum energy generation coil 521, and the fourth quantum energy generation coil 522, the minimum value (mT) and the maximum value (T) of the variable magnetic field, and the current value, voltage value, pulse width, pulse density adjustment, etc. supplied for each step of power supply time (second to minute or minute to time), and may be controlled by the following frequency: the frequency necessary for normalizing the kidney function; 1335 Hz, the frequency required for the normalization of the pituitary function; 635 Hz, the frequency required for the increase of the pituitary and the stimulation of normalization; 1725 Hz, 645 Hz, 1342 Hz, the frequency required for stimulation of the normality pineal gland function; 662 Hz, the frequency required for the normalization of the endocrine system function; 537 Hz, the frequency necessary for stimulation and normalization of immune system function; 835 Hz, the frequency required for stimulation of the normal colon function; 635 Hz, the frequency required for stimulation of the normal thyroid function; 763 Hz, the frequency required for the normalization of progesterone level (according to the level); 763 Hz, 1446 Hz, 1443 Hz, 763 Hz, the frequency required for the normalization (male and female) of the estrogen production level; 1351 Hz, the frequency required for the normalization (male) of the testosterone production level; 1444 Hz, the frequency required for the normalization (female) of the testosterone production level; 1445 Hz, the frequency required for stimulation of the normality pancreatic function; 654 Hz, the frequency required for stimulation of normal liver function; 751 Hz, the frequency required for stimulation of the normal kidney function; 625 Hz, the frequency required for stimulation of the normal heart function; 696 Hz, the frequency required for the normalization of the blood pressure; 15 Hz, the frequency required for stimulation of the normality nervous system function; 764 Hz, the frequency required for stimulation of phase lymphatic function; 676 Hz, the frequency required for stimulation of the increased lymphatic system circulation; 153 Hz, the frequency required for stimulation of the normalized blood circulation; 337 Hz, the frequency required for stimulation of increased blood volume/circulation; 17 Hz,
the frequency required for the normalization of the red blood cell production; 1524 Hz, the frequency required for the normalization of the white blood cell production; 1434 Hz, the frequency required the for hemoglobin production normalization; 2452 Hz, the frequency required for stimulation of the strengthening of the DNA conservation; 528 Hz, the frequency required for stimulation of the strengthening of the RNA conservation; Hz, 637 the frequency required for stimulation of the transparency of the thought (mental) function; 35 Hz, the frequency required for stimulation of the stability of the mood; 15 Hz, the frequency required for the emotional trauma removal stimulus; 15 Hz, the frequency required for the balance stimulation of well-being; 1565 Hz, the frequency required for the chemical substance hypersensitivity reduction; 440 Hz, the frequency required for the electromagnetic hypersensitivity reduction; 440 Hz, the frequency required for stimulation of the normalization of the calcium metabolism; 328 Hz, the frequency required for stimulation of neurotherapy; 2 Hz, the frequency required for stimulation of the bone treatment; 7 Hz, the frequency required for stimulation of the ligament treatment; 9.6 Hz, the frequency required for stimulation of the muscle treatment; 13.6 Hz, the frequency required for stimulation of the capillary treatment; 15.3 Hz, the frequency required for the intervertebral disc expansion reduction; 25.3, 324, 15 Hz, the frequency required for the excessive fluid retention reduction in the joint and organization; 24.3 Hz, the frequency required for the whole body back pain (fibromyositis pain syndrome) reduction; 326 Hz, the frequency of miracle, the DNA recovery, and the frequency required for the resonance between the human body and the nature; 528 Hz, the frequency required for the frequency which most accords with A′ nature of Verdi; 432 Hz, the frequency required to turn sorrow into joy and to freed from guilt and fear; 369 Hz, the frequency necessary for purifying trauma experience and promoting change; 417 Hz, the frequency necessary for the communications, understanding, patience and love promotion, and the strengthening relation; 639 Hz, the frequency required for delivery by the power of self-expression leading to a pure and stable life; 741 Hz, the frequency required to wake up intuition; 852 Hz, or the like. Any one of these frequencies is input to the input modules 513c-1 and 523c-1 by a user, and a plurality of parameters input by the user from the operation modules 513c-2 and 523c-2 are executed by an operation program, so that power for generating variable quantum energy is supplied to the first, second, third, and fourth quantum energy generation coils 511, 512, 521, and 522, a pulse-shaped magnetic field is generated at an angle of 90 degrees of a current flow, and the pulse-shaped magnetic fields in opposite directions are overlapped and offset (dissipated) at the center of a space (center of surfaces facing each other), thereby irradiating quantum energy generated in a zero magnetic field state to the solution of the first main body 501 and the second main body 502.

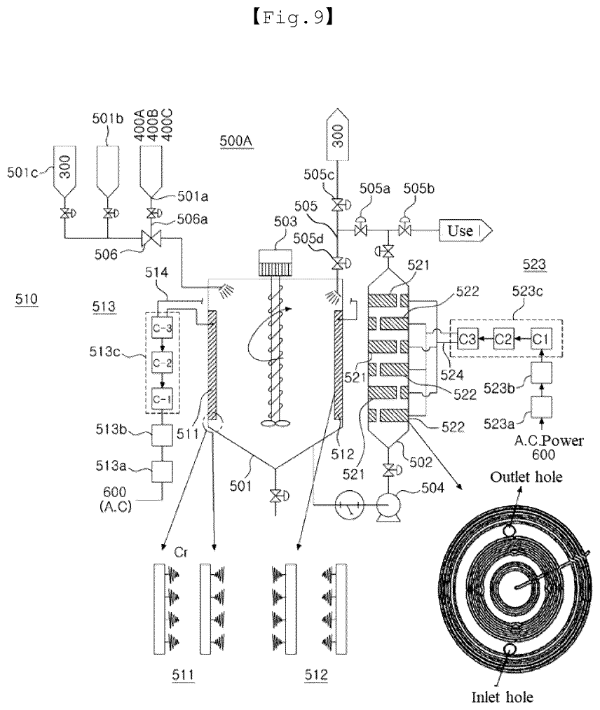

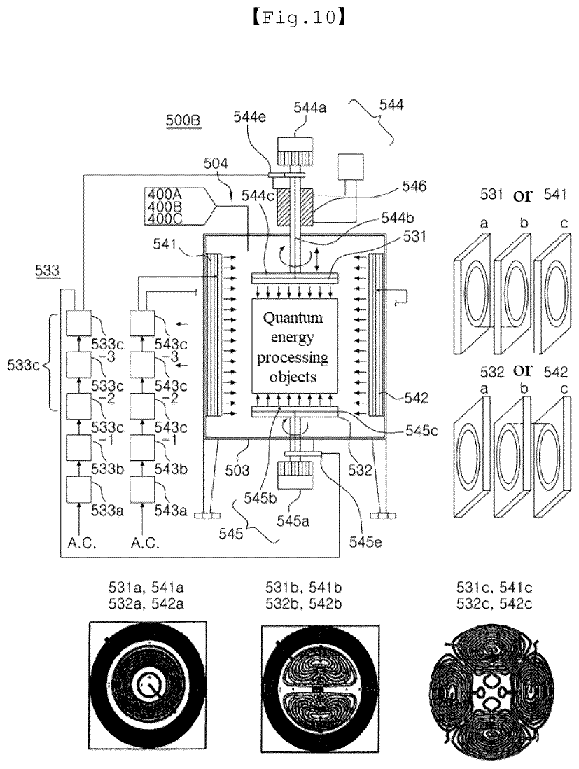

FIG. 10 is a schematic drawing illustrating the second quantum energy transfer device 500B of FIG. 1.

Referring to the accompanying drawings, the second quantum energy transfer device 500B includes a main body 503, an ion gas supply line 504, a first quantum energy generation device 530, an upper first rotating body motor 544, a pneumatic cylinder 544a, a first rotating disk 544b, a lower second rotating body motor 545, a second rotating disk 545b, and a second quantum energy generation device 540. The first quantum energy generation device 530 includes a first quantum energy generation coil 531, a second quantum energy generation coil 532, a first power supply unit 533, and a conducting wire 534. The second quantum energy generation device 540 includes a third quantum energy generation coil 541, a fourth quantum energy generation coil 542, a pulse-type power supply unit 543, and a conducting wire.

When the control panel 600 supplies power to the motor 545a for the second rotating body 545, the motor 544a for the first rotating body 544, and the pneumatic or hydraulic cylinder 546, the motor 545a of the second rotating body 545 is operated to rotate the motor shaft and the second rotating disk 545c.

At the same time, the pneumatic or hydraulic cylinder 546 installed at the center of the upper portion of the main body 503 is operated to lower the first rotating body 544 composed of the motor 544a, the shaft 544b, and the turntable 544c to the close distance (10 to 20 cm) of the quantum energy treatment object loaded on the turntable 545c of the second rotating body 545 inside the main body 503, and then the motor 544a of the first rotating body 544 is operated in the control panel to rotate the shaft 544b connected to the motor shaft and the turntable 544c to rotate the hydrogen ion (H⁻) or oxygen ions (O⁻) supplied from the first ion separator 400A, or oxygen ions (O⁺), the hydrogen ion (H⁺), the nitrogen ions (N⁺) or the nitrogen oxide ions (NO) supplied from the second ion separator 400B in a state where the ions are supplied or not supplied to one side of the upper portion of the main body 503 of the second quantum energy transfer device 500B by the pressing force of the air FAN 157. At the same time, the pulse-shaped electromagnetic fields generated by the first power supply unit 533 are irradiated from the upper portion to the lower portion and from the lower portion to the upper portion to the loaded quantum energy transfer target in the first quantum energy generation coil 531 of the plurality of first quantum energy generators installed to be in surface contact with the rear surface of the turntable 544c of the first rotating body 544 and the second quantum energy generation coil 532 installed to be in surface contact with the rear surface of the turntable 545c of the second rotating body 545 so that the pulse-shaped electromagnetic fields generated by the first power supply unit 533 are overlapped and offset (dissipated) in the center direction of the loaded target and transferred to the loaded object.

In addition, when the pulse-type power generated by the second power supply unit 543 is supplied to the first and second energy generating coils 541a, 541b, and 541c and the second energy generating coils 542a, 542b, and 542c of the plurality of second quantum energy generators, which are installed to face each other on the left and right side surfaces and the front and rear side surfaces inside the main body 503 of a rectangular parallelepiped shape and in which the winding directions of the coils are opposite to each other, through a conducting wire, electromagnetic fields having various frequencies in opposite directions are irradiated from the first and second energy generating coils, and at the same time, the electromagnetic fields in opposite directions are overlapped with and offset (dissipated) from the quantum energy transfer target installed at the center of the main body 503, so that pulse-type pulsating quantum energy corresponding to various frequencies generated in a zero magnetic field state is generated and transferred to the quantum energy transfer target.

In addition, as the turntable 545c of the second rotating device 545 rotates, a pulse-shaped electromagnetic field generated in the first and second quantum energy generation coils 541 and 542 of the first quantum energy generation device 530 installed on the inner side surface of the main body and a quantum energy generated by overlapping and dissipating the pulse-shaped electromagnetic field in opposite directions are uniformly irradiated to the quantum processing target.

The entire shapes of the first and second quantum energy generating coils 531 and 532 of the first quantum energy generator 530 and the first and second quantum energy generating coils 541 and 542 of the second quantum energy generator 540 may be the shape of the coils deformed on the surface of a rectangular or square plate.

Specifically, the shapes of the coils may be manufactured in such a manner that any one of Helmholtz coil, Cuffs coil, Trigger coil, Solenoid coil, and the shape in which one or more of these coils are combined is selected and attached to maintain a predetermined interval between the coil wound by a predetermined number of turns or is printed with a conductive ink or solution.

In addition, a plurality of the first and second quantum energy generation coils 531 and 532 of the first quantum energy generator 530 and a plurality of the third and fourth quantum energy generation coils 541 and 542 of the second quantum energy generator 540 are stacked as shown in FIG. 10. At this time, the winding direction of the coil in which the quantum energy is generated is the same direction in the plurality of stacked quantum energy generation coils. Also, winding directions of the first and second quantum energy generating coils 531 and 532 of the first quantum energy generators 530 installed facing each other are opposite to each other.

In addition, the winding directions of the third and fourth quantum energy generation coils 541 and 542 of the second quantum energy generator 540 are installed to be opposite to each other.

In addition, each base plate (not shown) on which the first and second quantum energy generation coils 531 and 532 of the first quantum energy generator 530 and the third and quantum energy generation coils 541 and 542 of the fourth second quantum energy generator 540 are installed has a rectangular flat plate shape, and may be manufactured by grinding the surface of an insulating plate selected from any one of PVC, PE, MC, acetal, and bakelite materials with an electric sanding machine, removing dust by a vacuum cleaner, washing the insulating plate with a cleaning agent such as ethyl alcohol or isopropyl alcohol, and conductive ink containing any one or more of silver, copper, and conductive carbon in an amount of 20% to 30% is selected from flexographic printing, screen printing, offset printing, glossy greener, inkjet printing, and dry printing methods, printed in a thickness of 10 μm to 30 μm in coils shape on the surface, and dried in a dryer (not shown) at a temperature of 50° C. to 80° C. for 8 hours to 12 hours.

In addition, any one coil shape may be selected from the shape of Solenoid coil, Toroid coil, Cuffs coil, Helmholtz coil, Gradient saddle coil, Uniform saddle coil, Trigger coil, Zigzag-shaped coil (up, down, left, and right), Temple-shaped coil, Stator-shaped coil, Toroidal coil, Flat coil, RF coil, Troidal coil, Tesla coil, Mobius coil, Caduceus coil, Rogoski coil, Helical coil, Resonant coil, and combination thereof, any one shape is selected as the selected shape.

And any one may be selected from the material of iron (Fe), copper (Cu), zinc (Zn), tin (Sn), stainless steel (STS 304, STS 316), aluminum (AL), titanium (Ti), platinum (Pt), and haloloid materials.

The first, second, third, and fourth quantum energy generating coils 531, 532, 541, and 542 may be processed into the selected coil shape using a laser and may be attached after an adhesive is applied to the surface of an insulating material.

In addition, any one coil shape may be selected from the shape of Solenoid coil, Toroid coil, Cuffs coil, Helmholtz coil, Gradient saddle coil, Uniform saddle coil, Trigger coil, Zigzag-shaped coil (up, down, left, and right), Temple-shaped coil, Stator-shaped coil, Toroidal coil, Flat coil, RF coil, Troidal coil, Tesla coil, Mobius coil, Caduceus coil, Rogoski coil, Helical coil, Resonant coil, and combination thereof, any one shape is selected as the selected shape. And the first, second, third, and fourth quantum energy generating coils 531, 532, 541, and 542 may be wound with a coated conductive metal wire in a shape selected after the adhesive material is applied to the surface of the insulating material.

The first power supply unit 533 applying the pulse power to the first and second quantum energy generation coils 531 and 532 includes a pressure reduction (pressure increase) transformer 533a, a rectifier circuit 533b, and a control unit 533c, the control unit 533c includes an input module 533c-1, a calculation module 533c-2, and a control module 533c-3 including a pulse width modulation (PWM) control method, a pulse frequency modulation (PFM), a pulse frequency (density) control (PDM), and a pulse repetition rate control (PRR) function.

When AC power of 220 V and 60 Hz is supplied to the pressure-increasing transformer 533a, the AC power is increased from the pressure-increasing transformer 533a to AC power of 1 to 10 kV and 60 Hz or reduced to AC power of 1 to 100 V and 60 Hz and supplied to the rectifier 533b, the AC power is converted from the rectifier 533b to DC power of 1 to 100 V or 1 to 10 kV.

The input module 533c-1 of the control unit 533c inputs, to a program embedded therein, variables such as a current value, a voltage value, a pulse width, a pulse density pulse period, a frequency burst length, a main power supply time and a stop time (timer function), a switching element function, and a surface temperature control function of the quantum energy generation coils 531 and 532, which are supplied to the first and second magnetic field generation coils 531 and 532, i.e.,
a minimum value mA and a maximum value A of the variable current flowing through the first and second magnetic field generation coils 531 and 532, the minimum value (mT) and the maximum value (T) of the variable magnetic field, the power supply time (seconds to minutes, or minutes to hours) for each step, and the like. Each of the parameters may be set on the monitor (not shown) of the input module 533c-1, for example, the operation step among the steps divided into steps 1 to 10, and then data of the selected step may be input.

The calculation module 533c-2 may calculate the amount of current corresponding to the intensity of the magnetic field to be generated in the first magnetic field generation coil 531 and the second magnetic field generation coil 532 for generating variable quantum energy by executing a calculation program on a plurality of parameters input by the user to the input module 533c-1, and may generate a current value by generating corresponding data in advance and matching the input frequency band, the magnetic field intensity value, and the current value one-to-one, measure the magnetic field of the first quantum energy generation coil 531 of the first quantum energy generator 530 by a magnetic field sensor (not shown), calculate a resonance frequency value capable of making a zero magnetic field state by overlapping the magnetic field generated in the first and second quantum energy generation coils 531 and 532 of the first quantum energy generator 530 with the data transmitted in real time, and generate a current value proportional to the calculated resonance frequency value. For example, when the intensity value of the magnetic field is between 1 and 10, data about a current value corresponding to between 1 and 10 is generated, and when the intensity value of the magnetic field is 1, a power of a pulsed electromagnetic field (PEMF) type having a current value corresponding to 1 is generated. In addition, the magnetic field sensor (not shown) measures the magnetic fields of the first and second quantum energy generation coils 531, 532 of the first quantum energy generator 530, and corrects the intensity value and current value of the magnetic field according to the data transmitted in real time.

The current control module 533c-1 generates a current corresponding to the strength of the magnetic field to be generated in the first magnetic field generating coil 531 and the second magnetic field generating coil 532 according to the current value transmitted from the calculation module 533c-2, supplies the current to the first quantum energy generating coil 531 and the second quantum energy generating coil 532, detects a current value flowing in one or each of the lead-in lines of the first and second quantum energy generating coils 531 and 532 by a current detection sensor (not shown) installed in one or each of the lead-in lines of the first and second quantum energy generating coils 531 and 532, and transmits the detected current value to the calculation module 533c-2 in real time. Then, the current control module 533c-1 compares the current value with a set value for each parameter input by a user, executes a calculation program when the current value deviates from the upper and lower limit values of the set value, and transmits a set value for each modified parameter so that they can be restored to the settings.

The current control module 533c-3 supplies the power of the pulse type (PEMF) suitable for the current modified for each step variable to the first quantum energy generation coil 531 and the second quantum energy generation coil 532, or the power of the pulse type (PEMF) suitable for the magnetic field value corresponding to the resonance frequency and the corrected current value corresponding to the resonance frequency by data transmitted by the magnetic field detection sensor (not shown) is supplied to the first quantum energy generation coil 531 and the second quantum energy generation coil 532, or the pulse type (PEMF) power is supplied to each of the first and second quantum energy generation coils.

In addition, the calculation module 533c-2 compares the generated current value with the received current value digitized from the first quantum energy generation coil 531 and the second quantum energy generation coil 532 received from the current detection sensor (not shown), and transmits a current value required through PID control according to the difference to the current control module 533c-3.

The magnetic field detection sensor (not shown) may be any one of a super conducting quantum interference device (SQUID) sensor, a nuclear magnetic resonance (NMR) sensor, an atomic magnetic resonance (AMR) sensor, a flux gate sensor, a magnetic resistance (MR) sensor, a magnetic impedance (MI) sensor, a hall effect sensor, an optical fiber magnetic sensor, and a search coil.

In the first power supply unit 533, the frequency modulation range may be in the range of 1 Hz to 100 Hz, 100 Hz to 1 kHz, 1 kHz to 10 kHz, 10 kHz to 1 MHz, 1 MHz to 100 MHz, and 100 MHz to 10 GHZ.

The second power supply unit 543 configured to apply the pulse power to the third and fourth quantum energy generation coils 541 and 542 includes a pressure-reduced transformer 543a, a rectifier circuit 543b, and a control unit 543c, the control unit 543c includes an input module 543c-1, a calculation module 543c-2, and a control module 543c-3 including a pulse width modulation (PWM) control method, a pulse frequency modulation PFM (pulse frequency modulation), a pulse frequency (density) control (PDM), and a pulse repetition rate control (PRR) function. When AC power of 220 V and 60 Hz is supplied to the pressure-increasing transformer 543a, the AC power is increased from the pressure-increasing transformer 543a to AC power of 1 to 10 kV and 60 Hz or reduced to AC power of 1 to 100 V and 60 Hz and supplied to the rectifier 543b, the AC power is converted from the rectifier 543b to DC power of 1 to 100 V or 1 to 10 kV.

The input module 543c-1 of the control unit 543c inputs, to a program embedded therein, variables such as a current value, a voltage value, a pulse width, a pulse density pulse period, a frequency burst length, a main power supply time and a stop time (timer function), a switching element function, and a strength of a magnetic field of the quantum energy generation coils 541 and 542, which are supplied to the first and second magnetic field generation coils 541 and 542, i.e., a minimum value mA and a maximum value A of the variable current flowing through the first and second magnetic field generation coils 541 and 542, the minimum value (mT) and the maximum value (T) of the variable magnetic field, the power supply time (seconds to minutes, or minutes to hours) for each step, and the like. Each of the parameters may be set on the monitor (not shown) of the input module 543c-1, for example, the operation step among the steps divided into steps 1 to 10, and then data of the selected step may be input.

The calculation module 543c-1 may calculate the amount of current corresponding to the intensity of the magnetic field to be generated in the first magnetic field generation coil 541 and the second magnetic field generation coil 542 for generating variable quantum energy by executing a calculation program on a plurality of parameters input by the user to the input module 543c-1, and may generate a current value by generating corresponding data in advance and matching the input frequency band, the magnetic field intensity value, and the current value one-to-one, measure the magnetic field of the first quantum energy generation coil 541 of the first quantum energy generator by a magnetic field sensor (not shown), calculate a resonance frequency value capable of making a zero magnetic field state by overlapping the magnetic field generated in the first and second quantum energy generation coils 541 and 542 of the second quantum energy generator 540 with the data transmitted in real time, and generate a current value proportional to the calculated resonance frequency value. For example, when the intensity value of the magnetic field is between 1 and 10, data about a current value corresponding to between 1 and 10 is generated, and when the intensity value of the magnetic field is 1, a power of a pulsed electromagnetic field (PEMF) type having a current value corresponding to 1 is generated. In addition, fields of the first and second quantum energy generation coils 541, 542 of the second quantum energy generator 540, and corrects the intensity value and current value of the magnetic field according to the data transmitted in real time.

The current control module 543c-1 generates a current corresponding to the strength of the magnetic field to be generated in the first magnetic field generating coil 541 and the second magnetic field generating coil 542 according to the current value transmitted from the calculation module 543c-2, supplies the current to the first quantum energy generating coil 541 and the second quantum energy generating coil 542, detects a current value flowing in one or each of the lead-in lines of the first and second quantum energy generating coils 541 and 542 by a current detection sensor (not shown) installed in one or each of the lead-in lines of the first and second quantum energy generating coils 541 and 542, and transmits the detected current value to the calculation module 543c-2 in real time. Then, the current control module 543c-1 compares the current value with a set value for each parameter input by a user, executes a calculation program when the current value deviates from the upper and lower limit values of the set value, and transmits a set value for each modified parameter so that they can be restored to the settings.

The current control module 543c-3 supplies the power of the pulse type (PEMF) suitable for the current modified for each step variable to the first quantum energy generation coil 541 and the second quantum energy generation coil 542, or the power of the pulse type (PEMF) suitable for the magnetic field value corresponding to the resonance frequency and the corrected current value corresponding to the resonance frequency by data transmitted by the magnetic field detection sensor (not shown) is supplied to the first quantum energy generation coil 541 and the second quantum energy generation coil 542 or the pulse type (PEMF) power is supplied to each of the first and second quantum energy generation coils.

In addition, the calculation module 543c-2 compares the generated current value with the received current value digitized from the first quantum energy generation coil 541 and the second quantum energy generation coil 542 received from the current detection sensor (not shown), and transmits a current value required through PID control according to the difference to the current control module 543c-3.

The items input to the input modules 533c-1 and 543c-1 of the control units 533c and 543c of the first power supply unit 533 and the second power supply unit 543 include the minimum (mA) and the maximum value (A) of the variable current of the power to be applied to the first quantum energy generation coil 531, the second quantum energy generation coil 532, the third quantum energy generation coil 541, and the fourth quantum energy generation coil 542, the minimum value (mT) and the maximum value (T) of the variable magnetic field, and the current value, voltage value, pulse width, pulse density adjustment, etc. supplied for each step of power supply time (second to minute or minute to time), and may be controlled by the following frequency: the frequency necessary for normalizing the kidney function; 1335 Hz, the frequency required for the normalization of the pituitary function; 635 Hz, the frequency required for the increase of the pituitary and the stimulation of normalization; 1725 Hz, 645 Hz, 1342 Hz, the frequency required for stimulation of the normality pineal gland function; 662 Hz, the frequency required for the normalization of the endocrine system function; 537 Hz, the frequency necessary for stimulation and normalization of immune system function; 835 Hz, the frequency required for stimulation of the normal colon function; 635 Hz, the frequency required for stimulation of the normal thyroid function; 763 Hz, the frequency required normalization of progesterone level (according to the level); 763 Hz, 1446 Hz, 1443 Hz, 763 Hz, the frequency required for the normalization (male and female) of the estrogen production level; 1351 Hz, the frequency required for the normalization (male) of the testosterone production level; 1444 Hz, the frequency required for the normalization (female) of the testosterone production level; 1445 Hz, the frequency required for stimulation of the normality pancreatic function; 654 Hz, the frequency required for stimulation of normal liver function; 751 Hz, the frequency required for stimulation of the normal kidney function; 625 Hz, the frequency required for stimulation of the normal heart function; 696 Hz, the frequency required for the normalization of the blood pressure; 15 Hz, the frequency required for stimulation of the normality nervous system function; 764 Hz, the frequency required for stimulation of phase lymphatic function; 676 Hz, the frequency required for stimulation of the increased lymphatic system circulation; 153 Hz, the frequency required for stimulation of the normalized blood circulation; 337 Hz, the frequency required for stimulation of increased blood volume/circulation; 17 Hz,
the frequency required for the normalization of the red blood cell production; 1524 Hz, the frequency required for the normalization of the white blood cell production; 1434 Hz, the frequency required for the hemoglobin production normalization; 2452 Hz, the frequency required for stimulation of the strengthening of the DNA conservation; 528 Hz, the frequency required for stimulation of the strengthening of the RNA conservation; 637 Hz, the frequency required for stimulation of the transparency of the thought (mental) function; 35 Hz, the frequency required for stimulation of the stability of the mood; 15 Hz, the frequency required for the emotional trauma removal stimulus; 15 Hz, the frequency required for the balance stimulation of well-being; 1565 Hz, the frequency substance required for the chemical hypersensitivity reduction; 440 Hz, the frequency required for the electromagnetic hypersensitivity reduction; 440 Hz, the frequency required for stimulation of the normalization of the calcium metabolism; 328 Hz, the frequency required for stimulation of neurotherapy; 2 Hz, the frequency required for stimulation of the bone treatment; 7 Hz, the frequency required for stimulation of the ligament treatment; 9.6 HZ, the frequency required for stimulation of the muscle treatment; 13.6 Hz, the frequency required for stimulation of the capillary treatment; 15.3 Hz, the frequency required for the intervertebral disc expansion reduction; 25.3, 324, 15 Hz, the frequency required for the excessive fluid retention reduction in the joint and organization; 24.3 Hz, the frequency required for the whole body back pain (fibromyositis pain syndrome) reduction; 326 Hz, the frequency of miracle, the DNA recovery, and the frequency required for the resonance between the human body and the nature; 528 Hz, the frequency required for the frequency which most accords with A′ nature of Verdi; 432 Hz, the frequency required to turn sorrow into joy and to freed from guilt and fear; 369 Hz, the frequency necessary for purifying trauma experience and promoting change; 417 Hz, the frequency necessary for the communications, understanding, patience and love promotion, and the strengthening relation; 639 Hz, the frequency required for delivery by the power of self-expression leading to a pure and stable life; 741 Hz, the frequency required to wake up intuition; 852 Hz, or the like. Any one of these frequencies is input to the input modules 533c-1 and 543c-1 by a user, and a plurality of parameters input by the user from the operation modules 533c-2 and 543c-2 are executed by an operation program, so that power for generating variable quantum energy is supplied to the first, second, third, and fourth quantum energy generation coils 531, 532, 541, and 552, a pulse-shaped magnetic field is generated at an angle of 90 degrees of a current flow, and the pulse-shaped magnetic fields in opposite directions are overlapped and offset (dissipated) at the center of a space (center of surfaces facing each other), thereby irradiating quantum energy generated in a zero magnetic field state to the quantum energy irradiation target loaded on the surface of the turntable 545c of the second rotating device 545 inside the first main body 503.

The object to be loaded on the surface of the turntable above (545c) has been any one or more of the following: processed meat such as beef, pork, chicken, rabbit, etc., various freshwater and sea fish, crustaceans such as crabs, shrimp, crayfish, and chicken shrimp, etc., grain such as rice, field rice, wheat, rye, oats, etc., and mixed grains such as corn, millet, blood, buckwheat, millet, etc., legumes such as beans, red beans, green beans, peas, kidney beans, etc., fruits such as persimmon, strawberry, pear, tangerine, mango, pineapple, orange, plum, cherry, quince, apricot, grape, kinkang, mulberry, melon, watermelon, peach, fig, hallabong, guava, banana, etc, vegetables such as lettuce, perilla leaf, crown daisy, cabbage, radish, cucumber, spinach, green onion, shilpa, onion, lettuce, eggplant, pumpkin, carrot, garlic, ginger, mushroom, leaf mustard, water parsley, red pepper, Angelica utilis, Lactuca sativa, chard, colabi, paprika, bell pepper, red beet, kale, parsley, etc., processed foods such as baking, confectionery, beverages, dairy products, meat processing, noodles, soy products, etc., food service industries such as liquor, coffee, tea, etc., seasoned foods such as soy sauce, vinegar, sauce, spices, kimchi, garlic, salt, butter, etc., raw material industries such as oils, starch, flour, sweeteners, etc., health foods, frozen foods, fermented foods, and seaweed such as green laver, kelp, seaweed fulvescens, sea grape, sea mustard, agar, fusiformis, glasswort, etc.

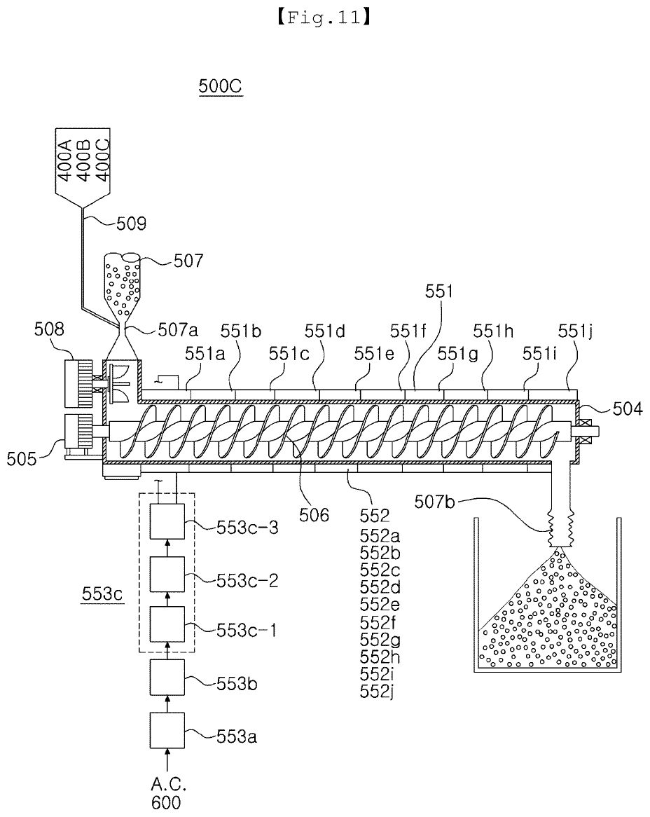

FIG. 11 is a schematic drawing illustrating the third quantum energy transfer device 500C of FIG. 1.

Referring to the accompanying drawings, the third quantum energy transfer device 500C includes a main body 504, a screw driving motor 505, a screw 506, a powder inlet 507, a powder outlet 507a, a supply fan 508, an ion gas supply line 509 and a third quantum energy generation device 550C. The third quantum energy generation device 550C includes a plurality of first quantum energy generation coils 551 (551a˜551j), a plurality of second quantum energy generation coils 552 (552a˜552j), a first power supply unit 553, and a conducting wire 554.

Hydrogen ions (H⁻), oxygen ions (O⁻), nitrogen ions (N⁻) or nitrogen oxide ions (NO) supplied from the first ion separator 400A, or hydrogen ions (H⁺), oxygen ions (O⁺) or nitrogen ions (N⁺) supplied from the second ion separator 400B, or hydrogen ions (H⁺) supplied from the third ion separator 400C may be supplied to the venturi neck part (powder outlet) 507a connected to the supply FAN 508 of the third quantum energy transfer device 500C by the suction power of the supply FAN 509 or by the pressing force of the air FAN 137, or there may be no supply of ions.

At the same time, when the quantum processing object (powder) is introduced into the main body 504 by the blade of the supply FAN 508, and discharged to the outlet 507a by the screw 506 driven by the drive motor 505′, the pulse-type power generated by the pulse-type power supply unit 553 is supplied through the wire to the multiple first quantum energy generating coils 551 and second quantum energy generating coils 552 installed to face each other in the circumferential direction of the outer circumferential surface of the main body 504, an electromagnetic field in the form of a pulse generated at a 90 degree angle in the current flow direction is irradiated from the top to the bottom of the main body 504 in the first quantum energy generating coil 551, and the second quantum energy generating coil 552 is irradiated from the bottom of the main body 504 to the top of the main body 504, and the pulse-type electromagnetic fields in opposite directions overlap and offset (dissipate) in the screw shaft portion to generate quantum energy in a zero magnetic field state.

When oxygen ions (O⁺, O⁻) are supplied from the ion gas supply line 509 while the generated quantum energy is irradiated to the quantum energy treatment object, the powder raw material may be mixed with oxygen ions (O⁺, O⁻) by screw driving inside the main body 504 to be perform an oxidation reaction.

When hydride ions (H⁻) or hydrogen ions (H⁺) are supplied from the ion gas supply line 509, the powder raw material may be mixed with hydride ions (H⁻) or hydrogen ions (H⁺) by screw driving inside the main body 504 to be perform a reduction reaction.

When nitrogen ions (N⁺, N⁻, NO) is supplied from the ion gas supply line 509, the powder raw material may be mixed with the nitrogen ions (N⁺, N⁻, NO) by screw driving inside the main body 504 to be perform a nitrification reaction.

As shown in FIG. 11, each of the first and second quantum energy generation coils 551 and 552 of the third quantum energy generator 550C is installed to face each other so that the winding directions of the coils are opposite to each other on the upper portion and the lower portion or both side surfaces of the cylindrical main body 504.

The first and second quantum energy generation coils 551 and 552 of the third quantum energy generator 550C may be selected from the shape of Solenoid coil, Toroid coil, Cuffs coil, Helmholtz coil, Gradient saddle coil, Uniform saddle coil, Trigger coil, Zigzag-shaped coil (up, down, left, and right), Temple-shaped coil, Stator-shaped coil, Toroidal coil, Flat coil, RF coil, Troidal coil, Tesla coil, Mobius coil, Caduceus coil, Rogoski coil, Helical coil, Resonant coil, and combination thereof, any one shape is selected. And any one may be selected from the material of iron (Fe), copper (Cu), zinc (Zn), tin (Sn), stainless steel (STS 304, STS 316), aluminum (AL), titanium (Ti), platinum (Pt), and haloloid materials. The first and second quantum energy generating coils 551 and 552 may be processed into the selected coil shape using a laser and may be attached after an adhesive is applied to the outer surface of the insulating main body 504.

In addition, any one coil shape may be selected from the shape of Solenoid coil, Toroid coil, Cuffs coil, Helmholtz coil, Gradient saddle coil, Uniform saddle coil, Trigger coil, Zigzag-shaped coil (up, down, left, and right), Temple-shaped coil, Stator-shaped coil, Toroidal coil, Flat coil, RF coil, Troidal coil, Tesla coil, Mobius coil, Caduceus coil, Rogoski coil, Helical coil, Resonant coil, and combination thereof, any one shape is selected as the selected shape, and the first and second quantum energy generating coils 551 and 552 may be wound with a coated conductive metal wire in a shape selected after the adhesive material is applied to the outer surface of the insulating main body 504.

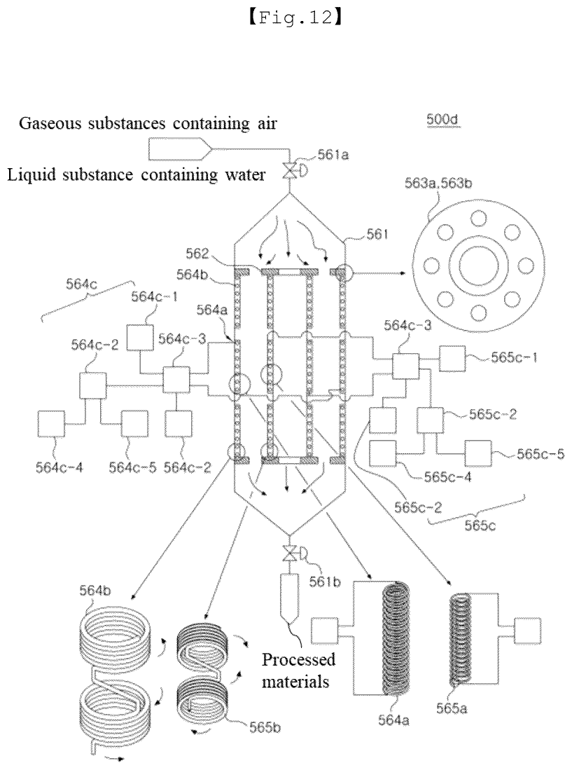

The shape of the quantum energy generating coil is preferably a modified solenoid coil shape or an inclined coil shape and Gradient uniform saddle shape as shown in FIG. 12.

The first power supply unit 553 applying the pulse power to the first and second quantum energy generation coils 551 and 552 includes a pressure reduction (pressure increase) transformer 553a, a rectifier circuit 553b, and a control unit 553c, the control unit 553c includes an input module 553c-1, a calculation module 553c-2, and a control module 553c-3 including a pulse width modulation (PWM) control method, a pulse frequency modulation (PFM), a pulse frequency (density) control (PDM), and a pulse repetition rate control (PRR) function.

When AC power of 220 V and 60 Hz is supplied to the pressure-increasing transformer 553a, the AC power is increased from the pressure-increasing transformer 553a to AC power of 1 to 10 kV and 60 Hz or reduced to AC power of 1 to 100 V and 60 Hz and supplied to the rectifier 553b, the AC power is converted from the rectifier 553b to DC power of 1 to 100 V or 1 to 10 kV.

The input module 553c-1 of the control unit 553c inputs, to a program embedded therein, variables such as a current value, a voltage value, a pulse width, a pulse density pulse period, a frequency burst length, a main power supply time and a stop time (timer function), a switching element function, and a strength of a magnetic field of the quantum energy generation coils 551 and 552, which are supplied to the first and second magnetic field generation coils 551 and 552, i.e., a minimum value mA and a maximum value A of the variable current flowing through the first and second magnetic field generation coils 551 and 552, the minimum value (mT) and the maximum value (T) of the variable magnetic field, the power supply time (seconds to minutes, or minutes to hours) for each step, and the like. Each of the parameters may be set on the monitor (not shown) of the input module 553c-1, for example, the operation step among the steps divided into steps 1 to 10, and then data of the selected step may be input.

The calculation module 553c-2 may calculate the amount of current corresponding to the intensity of the magnetic field to be generated in the first magnetic field generation coil 551 and the second magnetic field generation coil 552 for generating variable quantum energy by executing a calculation program on a plurality of parameters input by the user to the input module 553c-1, and may generate a current value by generating corresponding data in advance and matching the input frequency band, the magnetic field intensity value, and the current value one-to-one, measure the magnetic field of the first quantum energy generation coil 551 of the first quantum energy generator 550 by a magnetic field sensor (not shown), calculate a resonance frequency value capable of making a zero magnetic field state by overlapping the magnetic field generated in the first and second quantum energy generation coils 551 and 552 of the first quantum energy generator 550 with the data transmitted in real time, and generate a current value proportional to the calculated resonance frequency value. For example, when the intensity value of the magnetic field is between 1 and 10, data about a current value corresponding to between 1 and 10 is generated, and when the intensity value of the magnetic field is 1, a power of a pulsed electromagnetic field (PEMF) type having a current value corresponding to 1 is generated. In addition, fields of the first and second quantum energy generation coils 551, 552 of the first quantum energy generator 550, and corrects the intensity value and current value of the magnetic field according to the data transmitted in real time.

The current control module 553c-1 generates a current corresponding to the strength of the magnetic field to be generated in the first magnetic field generating coil 551 and the second magnetic field generating coil 552 according to the current value transmitted from the calculation module 553c-2, supplies the current to the first quantum energy generating coil 551 and the second quantum energy generating coil 552, detects a current value flowing in one or each of the lead-in lines of the first and second quantum energy generating coils 551 and 552 by a current detection sensor (not shown) installed in one or each of the lead-in lines of the first and second quantum energy generating coils 551 and 552, and transmits the detected current value to the calculation module 553c-2 in real time. Then, the current control module 553c-1 compares the current value with a set value for each parameter input by a user, executes a calculation program when the current value deviates from the upper and lower limit values of the set value, and transmits a set value for each modified parameter so that they can be restored to the settings.

The current control module 553c-3 supplies the power of the pulse type (PEMF) suitable for the current modified for each step variable to the first quantum energy generation coil 551 and the second quantum energy generation coil 552, or the power of the pulse type (PEMF) suitable for the magnetic field value corresponding to the resonance frequency and the corrected current value corresponding to the resonance frequency by data transmitted by the magnetic field detection sensor (not shown) is supplied to the first quantum energy generation coil 551 and the second quantum energy generation coil 552, or the pulse type (PEMF) power is supplied to each of the first and second quantum energy generation coils.

In addition, the calculation module 553c-2 compares the generated current value with the received current value digitized from the first quantum energy generation coil 551 and the second quantum energy generation coil 552 received from the current detection sensor (not shown), and transmits a current value required through PID control according to the difference to the current control module 553c-3.

The magnetic field detection sensor (not shown) may be any one of a super conducting quantum interference device (SQUID) sensor, a nuclear magnetic resonance (NMR) sensor, an atomic magnetic resonance (AMR) sensor, a flux gate sensor, a magnetic resistance (MR) sensor, a magnetic impedance (MI) sensor, a hall effect sensor, an optical fiber magnetic sensor, and a search coil.

In the first power supply unit 553, the frequency modulation range may be in the range of 1 Hz to 100 Hz, 100 Hz to 1 kHz, 1 kHz to 10 kHz, 10 kHz to 1 MHz, 1 MHz to 100 MHz, and 100 MHz to 10 GHZ.

The items input to the input module 553c-1 of the control unit 553c of the first power supply unit include the minimum (mA) and the maximum value (A) of the variable current of the power to be applied to the first quantum energy generation coil 551 and the second quantum energy generation coil 552, the minimum value (mT) and the maximum value (T) of the variable magnetic field, and the current value, voltage value, pulse width, pulse density adjustment, etc. supplied for each step of power supply time (second to minute or minute to time), and may be controlled by the following frequency: the frequency necessary for normalizing the kidney function; 1335 Hz, the frequency required for the normalization of the pituitary function; 635 Hz, the frequency required for the increase of the and pituitary the stimulation of normalization; 1725 Hz, 645 Hz, 1342 Hz, the frequency required for stimulation of the normality pineal gland function; 662 Hz, the frequency required for the normalization of the endocrine system function; 537 Hz, the frequency necessary for stimulation and normalization of immune system function; 835 Hz, the frequency required for stimulation of the normal colon function; 635 Hz, the frequency required for stimulation of the normal thyroid function; 763 Hz, the frequency required for the normalization of progesterone level (according to the level); 763 Hz, 1446 Hz, 1443 Hz, 763 Hz, the frequency required for the normalization (male and female) of the estrogen production level; 1351 Hz, the frequency required for the normalization (male) of the testosterone production level; 1444 Hz, the frequency required for the normalization (female) of the testosterone production level; 1445 Hz, the frequency required for stimulation of the normality pancreatic function; 654 Hz, the frequency required for stimulation of normal liver function; 751 Hz, the frequency required for stimulation of the normal kidney function; 625 Hz, the frequency required for stimulation of the normal heart function; 696 Hz, the frequency required for the normalization of the blood pressure; 15 Hz, the frequency required for stimulation of the normality nervous system function; 764 Hz, the frequency required for stimulation of phase lymphatic function; 676 Hz, the frequency required for stimulation of the increased lymphatic system circulation; 153 Hz, the frequency required for stimulation of the normalized blood circulation; 337 Hz, the frequency required for stimulation of increased blood volume/circulation; 17 Hz,
the frequency required for the normalization of the red blood cell production; 1524 Hz, the frequency required for the normalization of the white blood cell production; 1434 Hz, the frequency required hemoglobin production for the normalization; 2452 Hz, the frequency required for stimulation of the strengthening of the DNA conservation; 528 Hz, the frequency required for stimulation of the strengthening of the RNA conservation; 637 Hz, the frequency required for stimulation of the transparency of the thought (mental) function; 35 Hz, the frequency required for stimulation of the stability of the mood; 15 Hz, the frequency required for the emotional trauma removal stimulus; 15 Hz, the frequency required for the balance stimulation of well-being; 1565 Hz, the frequency required for the chemical substance hypersensitivity reduction; 440 Hz, the frequency required for the electromagnetic hypersensitivity reduction; 440 Hz, the frequency required for stimulation of the normalization of the calcium metabolism; 328 Hz, the frequency required for stimulation of neurotherapy; 2 Hz, the frequency required for stimulation of the bone treatment; 7 Hz, the frequency required for stimulation of the ligament treatment; 9.6 Hz, the frequency required for stimulation of the muscle treatment; 13.6 Hz, the frequency required for stimulation of the capillary treatment; 15.3 Hz, the frequency required for the intervertebral disc expansion reduction; 25.3, 324, 15 Hz, the frequency required for the excessive fluid retention reduction in the joint and organization; 24.3 Hz, the frequency required for the whole body back pain (fibromyositis pain syndrome) reduction; 326 Hz, the frequency of miracle, the DNA recovery, and the frequency required for the resonance between the human body and the nature; 528 Hz, the frequency required for the frequency which most accords with A′ nature of Verdi; 432 Hz, the frequency required to turn sorrow into joy and to freed from guilt and fear; 369 Hz, the frequency necessary for purifying trauma experience and promoting change; 417 Hz, the frequency necessary for the communications, understanding, patience and love promotion, and the strengthening relation; 639 Hz, the frequency required for delivery by the power of self-expression leading to a pure and stable life; 741 Hz, the frequency required to wake up intuition; 852 Hz, or the like. Any one of these frequencies is input to the input module 553c-1 by a user, and a plurality of parameters input by the user from the operation module 553c-2 are executed by an operation program, so that power for generating variable quantum energy is supplied to the first, second, third, and fourth quantum energy generation coils 551 and 552, a pulse-shaped magnetic field is generated at an angle of 90 degrees of a current flow, and the pulse-shaped magnetic fields in opposite directions are overlapped and offset (dissipated) at the center of a space (center of surfaces facing each other), thereby irradiating quantum energy generated in a zero magnetic field state to the powder raw material passing through the inside the first main body 504.

The powder raw material passing through the inside of the main body 504 may be any one or more selected from peanut, soybean, macadamia, almond, coffee beans, ginkgo nut, watermelon seeds, etc., pine nuts, cacao, cashew nuts, coconuts, tiger nuts, pastachios, sunflower seeds, walnuts, pumpkin seeds, rice, barley, wheat, sorghum, or their powder, and may be introduced into and passed through the inside of the main body 504.

FIG. 12 is a schematic drawing illustrating a fourth quantum energy transfer device 500D of FIG. 1.

Referring to the accompanying drawings, the fourth quantum energy transfer device 500D may be installed independently without being connected to the raw material supply unit 100, the ion generator 200, the dissolving device 300, the ion separator 400, and the quantum energy transfer devices 500 (500A, 500B, 500C).

The fourth quantum energy transfer device 500D includes a first main body 561, a second main body (an inner cylinder) 562, upper and lower fixed disks 563a and 563b, a first quantum energy generator 560 including a first electromagnetic energy generator 564 and a second electromagnetic energy generator 565, and electric valves installed on a line of an inlet 561a and an outlet 561b.

The first electromagnetic energy generator 564 includes a first transmitting coil 564a, first receiving coils 564b, and a power supply unit 564c having an RF signal generation function. And the second electromagnetic energy generator 565 includes a second transmitting coil 565a, second receiving coils 565b, and a power supply unit 565c having an RF signal generation function.

The upper fixed disk 563a and the lower fixed disk 563b may be installed to be spaced apart from each other at a certain distance on one upper side and one lower side of the first main body 561.

The upper fixed disk 563a and the lower fixed disk 563b may have a shape in which a plurality of holes having a predetermined diameter are perforated in the circumferential direction.

Materials of the upper and lower fixed disks 563a and 563b may be one of a Teflon material, a polyethylene (PE), a PVC material, and an MC material.

After a varnish made of an insulating material is applied and cured on the circumferential surface of the inner surface of the first main body 561, a predetermined number of the first transmitting coil 564a are wound around the central portion of the inner surface of the first main body 561 at a predetermined height, the first receiving coils 564b may be installed above and below the first transmitting coil 564a at predetermined intervals. A power supply unit 564c having an RF signal generation function for supplying power to the first transmitting coil 564a through a conducting wire may be installed in the center of the outer surface of the first main body 561.

In addition, The second main body 562 having a predetermined diameter and length may be installed at a central portion of the first main body 561 to be spaced apart from the main body by a predetermined distance in a hole perforated at central portions of the upper and lower fixing plates 563a and 563b, the second transmitting coil 565a and the second receiving coils 565b of a second electromagnetic energy generator 565 may be installed on an outer surface of the second main body 562 to face each other at the same height at which the first transmitting coil 564a and the first receiving coils 564b of the first electromagnetic energy generator 564 installed on an inner surface of the first main body 561 may be installed, and a second power supply unit 565c of an RF signal generating function may be installed at a central portion of an outer surface of the first main body 561 to be spaced apart from the first power supply unit 564c by a predetermined distance. An output side of the second power supply unit 565c is connected to the second transmission coil 565a with a conductive wire.

The winding directions of the first transmitting coil 564a of the first electromagnetic energy generator 564 and the second transmitting coil 565a of the second electromagnetic energy generator 565 are opposite to each other. Also, the winding directions of the first receiving coils 564b of the first electromagnetic energy generator 564 and the second receiving coils 565b of the second electromagnetic energy generator 565 are opposite to each other.

The winding directions of the first transmitting coil 564a and the first receiving coils 564b of the first electromagnetic energy generator 564 are opposite to each other, and the winding directions of the second transmitting coil 565a and the second receiving coils 565b of the second electromagnetic energy generator 565 are opposite to each other.

The first transmitting coil 564a and the first receiving coils 564b of the first electromagnetic energy generator 564 may be any one of the shapes of Solenoid coil, Helical coil, and Trigger coil, and RF signal generation power generated by the first power supply unit 565c may be supplied only to the first transmitting coil 564a by a conducting wire, and the first transmitting coil 564a and the first receiving coils 564b may have a resonance coil structure.

The second transmitting coil 565a and the second receiving coils 565b of the second electromagnetic energy generator 565 may be any one of the shapes of Solenoid coil, Helical coil, the RF signal generation function power generated by the second power supply unit 565c may be supplied only to the second transmitting coil 565a as a conducting wire, and the second transmitting coil 565a and the second receiving coils 565b may have a resonance coil structure.

The first power supply unit 564C of the first electromagnetic energy generator 564 may include a pulse-type variable frequency RF generator 564c-1, a duty cycle controller 564c-2 in which an alternating off-regulator 564c-5 and an alternating on-regulator 564c-4 are embedded, a power amplifier 564c-3, and a power/amplitude controller 564c-4.

The power amplifier 564c-3 controlled by the power/amplitude controller 564c-2 operates as an input of the RF signal A from the pulse-type variable frequency RF generator 564c-1, and outputs signals with variable amplitudes according to the selected processing parameter to the first transmitting coil 564a through the RF-modulated signal B.

When power is amplified by the power amplifier 564c-3 of the first power supply unit 564c and the RF modulated signal having an adjusted frequency amplitude is supplied to the first transmitting coil 564a, the first receiving coils 564b spaced apart from the first transmitting coil 564a by a predetermined distance in the vertical direction and wound by a predetermined number of turns is resonated to generate an electromagnetic field at an angle of 90 degrees in the current flow direction of the first transmitting coil 564a and the first receiving coils 564b, thereby irradiating electromagnetic waves to a gaseous material including air or a liquid material including water passing through a flow path formed between the first main body 561 and the second main body 562.

In addition, the duty cycle controller 564c-2 controls the power amplifier 564c-3 by changing the output to on and off. The duty cycle controller 564c-2 is set according to the on-regulator 564c-4 and the off-regulator 564c-5 to generate a duty cycle according to the selected processing parameter. The on-regulator 564c-5 and the off-regulator 564c-5 may be preferably adjusted with a duration ranging from 1 minute to 60 minutes, for example. Therefore, a normal setting of turning on for 10 minutes and turning off for 60 minutes may be used.

A number of sensors (not shown) may be provided to allow for confirmation of the efficiency for setting processing parameters in a real environment.

However, the sensors (not shown) may be replaced with sensors that directly determine changes to the solution, i.e., zeta potential values of the solution. In this alternative embodiment, the potential processor (not shown) may change the duty cycle controller 564c-2 and the power amplifier 564c-3 by sensing the change made for the solution, the other potential, and actively setting the desired setting values for the alternating on-regulator 564c-5, the alternating off-regulator 564c-5, and power amplifier 564c-3.

The second power supply unit 565c of the second electromagnetic energy generator 565 may include a pulse-type variable frequency RF generator 565c-1, a duty cycle controller 565c-2 in which an alternating on-regulator 565c-4 and an alternating off-regulator 565c-5 are embedded, a power amplifier 565c-3, and a power/amplitude adjuster 565c-4.

The power amplifier 565c-3 controlled by the power/amplitude controller 565c-2 operates as an input of the RF signal A from the pulse-type variable frequency RF generator 565c-1, and outputs signals with variable amplitudes according to the selected processing parameter to the second transmitting coil 565a through the RF-modulated signal B.

When power is amplified by the power amplifier 565c-3 of the second power supply unit 565c and the RF modulated signal having an adjusted frequency amplitude is supplied to the second transmitting coil 565a, the second receiving coils 565b spaced apart from the second transmitting coil 565a by a predetermined distance in the vertical direction and wound by a predetermined number of turns is resonated to generate an electromagnetic field at an angle of 90 degrees in the current flow direction of the second transmitting coil 565a and the second receiving coils 565b, thereby irradiating electromagnetic waves to a gaseous material including air or a liquid material including water passing through a flow path formed between the first main body 561 and the second main body 562.

In addition, the duty cycle controller 565c-2 controls the power amplifier 565c-3 by changing the output to on and off. The duty cycle controller 565c-2 is set according to the on-regulator 565c-4 and the off-regulator 565c-5 to generate a duty cycle according to the selected processing parameter. The on-regulator 565c-5 and the off-regulator 565c-5 may be preferably adjusted with a duration ranging from 1 minute to 60 minutes, for example. Therefore, a normal setting of turning on for 10 minutes and turning off for 60 minutes may be used.

A number of sensors (not shown) may be provided to allow for confirmation of the efficiency for setting processing parameters in a real environment.

However, the sensors (not shown) may be replaced with sensors that directly determine changes to the solution, i.e., zeta potential values of the solution. In this alternative embodiment, the potential processor (not shown) may change the duty cycle controller 565c-2 and the power amplifier 565c-3 by sensing the change made for the solution, the other potential, and actively setting the desired setting values for the alternating on-regulator 565c-5, the alternating off-regulator 565c-5, and the power amplifier 565c-3.

Gaseous materials containing air, or liquid materials containing water from the supply pipe at the top of the first body 561 passes through the electronic valve 561a under the pressure of the fluid machine (not shown) and through the flow path formed between the first body 561 and the second body 562 in a double cylindrical shape.

In this process, when the frequency generated by the first power supply device 564c of the first electromagnetic energy generator 564 is varied and RF energy such as a pulsed electric field and a magnetic field is applied to the first transmitting coil 564a, the pulsed electric field and magnetic field energy (variable RF energy) generated at a 90 degree angle in the current flow direction are irradiated to the gaseous materials containing air passing through the flow path or the liquid materials containing water passing through the flow path.

At the same time, the first receiving coils 564b also resonates with the first transmitting coil 564a to irradiate pulsed electric and magnetic field energy (variable RF energy) to gaseous materials containing air passing through the flow path or liquid materials containing water passing through the flow path.

In addition, when the frequency generated by the second power supply unit 565c of the second electromagnetic energy generator 565 is varied and RF energy such as a pulse-shaped electric field and a magnetic field is applied to the second transmitting coil 565a, the pulse-shaped electric field and the magnetic field energy (variable RF energy) generated at an angle of 90 degrees with respect to the current flow direction are irradiated to the gaseous materials including air passing through the flow path or the liquid materials including water through the flow path, and at the same time, the second receiving coils 565b resonates with the second transmitting coil 565a to irradiate the pulse-shaped electric field and the magnetic field energy (variable RF energy) to the gaseous materials including air passing through the flow path or the liquid materials including water through the flow path.

In addition, since the winding directions of the first transmitting coil 564a and the first receiving coils 564b of the first electromagnetic energy generator 564 and the winding directions of the second transmitting coil 565a and the second receiving coils 565b of the second electromagnetic energy generator 565 are opposite to each other, electric fields and magnetic field energy (variable RF energy) generated from the first transmitting coil 564a and the first receiving coils 564b resonated with the first transmitting coil 564a are overlapped and offset (dissipated) with each other at the center of the flow path formed between the first main body 561 and the second main body 562. Also, the electric fields and magnetic field energy (variable RF energy) generated from the second transmitting coil 565a and the second receiving coils 565b resonated with second transmitting coil 565a are overlapped and offset (dissipated) with each other at the center of the flow path formed between the first main body 561 and the second main body 562, and pulse-shaped pulsating quantum energy generated extinguished state is irradiated to the gaseous materials including air passing through the flow path or the liquid materials including water through the flow path.

The intensity of the electric field and magnetic energy (RF energy) generated by the first transmitting coil 564a and the receiving coils 564b, and the intensity of the electric field and magnetic energy (RF energy) generated by the second transmitting coil 565a and the second receiving coils 565b may be as follows;
the field electron energy (1E, eV) capable of decomposing the covalent bond of oxygen molecules (O₂) in contaminated air is 12.0857 eV or more, the electric field electron energy (1E, eV) capable of decomposing the covalent bond of water molecules of water vapor is 12.621 eV or higher, the field electron energy (1E, eV) capable of decomposing the propionic acid molecule (C₃H₆O₂) covalent bond is greater than or equal to 10.44 eV, the field electron energy (1E, eV) capable of decomposing the covalent bond of hydrogen sulfide (H₂S) molecules is 10.457 eV or more, the field electron energy (1E, eV) capable of decomposing the covalent bond of the methylmercaptan molecule is greater than or equal to 9.439 eV, the field electron energy (1E, eV) capable of decomposing the covalent bond of the acetaldehyde molecule is greater than or equal to 10.229 eV, the field electron energy (1E, eV) capable of decomposing the covalent bond of methylethylketone molecules is greater than or equal to 9.52 eV, the field electron energy (1E, eV) capable of decomposing the covalent bond of methylamine molecules is greater than or equal to 8.9 ev, the field electron energy (1E, eV) capable of decomposing the covalent bond of the ethylamine molecule is greater than or equal to 8.9 eV, the field electron energy (1E, eV) capable of decomposing the covalent bonds of triethylamine molecules is greater than or equal to 7.53 eV, the field electron energy (1E, eV) capable of decomposing the covalent bonds of ammonia molecules is 10.57 eV or more, the field electron energy (1E, eV) capable of decomposing the covalent bond of the acetic acid molecule is 10.95 eV or more, the field electron energy (1E, eV) capable of decomposing the covalent bond of the molecule of propionic acid is greater than or equal to 10.44 ev, the field electron energy (1E, eV) capable of decomposing the covalent bond of valeric acid molecules is greater than or equal to 10.53 eV, the field electron energy (1E, eV) capable of decomposing the covalent bond of the dimethyl sulfide molecule is greater than or equal to 7.4 eV, the field electron energy (1E, eV) capable of decomposing the covalent bond of the butylaldehyde molecule is greater than or equal to 10.53 ev, the field electron energy (1E, eV) capable of decomposing the covalent bond of the MIBK molecule is greater than or equal to 9.3 eV, the field electron energy (1E, eV) capable of decomposing the covalent bond of ethylbenzene molecules is greater than or equal to 8.77 eV, the field electron energy (1E, eV) capable of decomposing the covalent bond of the xylene molecule is greater than or equal to 8.55 eV, and the field electron energy (1E, eV) capable of decomposing the covalent bond of the carbon disulfide (CS₂) molecules is greater than or equal to 10.073 eV.

The dissociation energy of the interatomic covalent bond that breaks the bond between atoms after the covalent bond of the material molecule is dissociated is required as follows; 414 KJ/mol or more for C—H bonds, 389 KJ/mol or more for N—H bonds, 348 KJ/mol or more for O—H bonds, 163 KJ/mol or more for N—N bonds, 146 KJ/mol or more for 0-0 bonds, 253 KJ/mol or more for Cl—F bonds, 293 KJ/mol or more for C—N bonds, 201 kJ/mol or more for N—O bonds, 190 KJ/mol or more for O—F bonds, 242 KJ/mol or more for Cl—Cl bonds, 351 KJ/mol or more for C—O bonds, 272 KJ/mol or more for N—F bonds, 203 KJ/mol or more for O—Cl bonds, 237 KJ/mol or more for Br—F bonds, 439 kJ/mol or more for C—F bonds, 200 KJ/mol or more for N—Cl bonds, 234 KJ/mol or more for O—I bonds, 218 kJ/mol or more for Br—Cl bonds, 611 KJ/mol or more for C═C bonds, 837 KJ/mol or more for C═C bonds, 615 KJ/mol or more for C═N bonds, and 799 KJ/mol or more for C═O bonds. [Source: Chemical-covalent bonding energy, author Fleshming]

In addition, it should be possible to form a potential difference of 1V or more capable of killing microorganisms present in a liquid substance including water, and the related contents are as follows.

A method of killing (destroying) the membrane of a microorganism by electroporation is disclosed.

It can be inferred from the microorganism removal process of Zimmerman's research results. Zimmerman published the research results that if there is a potential difference of about 1 volt around the cell membrane of the microorganism, the microorganism membrane is broken down, and the contents in the cell flow out of the cell to kill the microorganism. (Zimmerman, U., G. Pilwat, and F. Eiemann, Dielectric Breakdown of cell membranes, Biophys. J. 1974 November; 14(11):88199).

Electroporation refers to the fact that the plasma membrane of cells exposed to a high-voltage pulsed electric field in certain specific parameters is temporarily permeable due to the destabilization of the lipid bilayer and the formation of pores.

The cell plasma membrane consists of a lipid bilayer of approximately 5 nm thickness.

The cell membrane essentially acts as a non-conductive dielectric barrier that forms a capacitor. Even when there is no applied electric field due to physiological conditions, a potential difference naturally occurs due to a charge separation phenomenon formed across the cell membrane between the inside and the outside of the cell membrane.

In addition, the items input to the first power supply unit 564c of the first electromagnetic energy generator 564, the second power supply unit 565c of the second electromagnetic energy generator 565, the pulse-type variable frequency RF generator 564c-1 of the first power supply unit 564c, the duty cycle controller 564c-2, and the power/amplitude controller 564c-4 include the minimum (mA) and the maximum value (A) of the variable current of the power to be applied to the first transmitting coil 564a and the second transmitting coil 565a, the minimum value (mT) and the maximum value (T) of the variable magnetic field, and the current value, voltage value, pulse width, pulse density adjustment, etc. supplied for each step of power supply time (second to minute or minute to time), and may be controlled by the following frequency: the frequency necessary for normalizing the kidney function; 1335 Hz, the frequency required for the normalization of the pituitary function; 635 Hz, the frequency required for the increase of the pituitary and the stimulation of normalization; 1725 Hz, 645 Hz, 1342 Hz, the frequency required for stimulation of the normality pineal gland function; 662 Hz, the frequency required for the normalization of the endocrine system function; 537 Hz, the frequency necessary for stimulation and normalization of immune system function; 835 Hz, the frequency required for stimulation of the normal colon function; 635 Hz, the frequency required for stimulation of the normal thyroid function; 763 Hz, the frequency required for the normalization of progesterone level (according to the level); 763 Hz, 1446 Hz, 1443 Hz, 763 Hz, the frequency required for the normalization (male and female) of the estrogen production level; 1351 Hz, the frequency required for the normalization (male) of the testosterone production level; 1444 Hz, the frequency required for the normalization (female) of the testosterone production level; 1445 Hz, the frequency required for stimulation of the normality pancreatic function; 654 Hz, the frequency required for stimulation of normal liver function; 751 Hz, the frequency required for stimulation of the normal kidney function; 625 Hz, the frequency required for stimulation of the normal heart function; 696 Hz, the frequency required for the normalization of the blood pressure; 15 Hz, the frequency required for stimulation of the normality nervous system function; 764 Hz, the frequency required for stimulation of phase lymphatic function; 676 Hz, the frequency required for stimulation of the increased lymphatic system circulation; 153 Hz, the frequency required for stimulation of the normalized blood circulation; 337 Hz, the frequency required for stimulation of increased blood volume/circulation; 17 Hz,
the frequency required for the normalization of the red blood cell production; 1524 Hz, the frequency required for the normalization of the white blood cell production; 1434 Hz, the frequency required for the hemoglobin production normalization; 2452 Hz, the frequency required for stimulation of the strengthening of the DNA conservation; 528 Hz, the frequency required for stimulation of the strengthening of the RNA conservation; 637 HZ, frequency required for stimulation of the transparency of the thought (mental) function; 35 Hz, the frequency required for stimulation of the stability of the mood; 15 Hz, the frequency required for the emotional trauma removal stimulus; 15 Hz, the frequency required for the balance stimulation of well-being; 1565 Hz, the frequency required for the chemical substance hypersensitivity reduction; 440 Hz, the frequency required for the electromagnetic hypersensitivity reduction; 440 Hz, the frequency required for stimulation of the normalization of the calcium metabolism; 328 Hz, the frequency required for stimulation of neurotherapy; 2 Hz, the frequency required for stimulation of the bone treatment; 7 Hz, the frequency required for stimulation of the ligament treatment; 9.6 Hz, the frequency required for stimulation of the muscle treatment; 13.6 Hz, the frequency required for stimulation of the capillary treatment; 15.3 Hz, the frequency required for the intervertebral disc expansion reduction; 25.3, 324, 15 Hz, the frequency required for the excessive fluid retention reduction in the joint and organization; 24.3 Hz, the frequency required for the whole body back pain (fibromyositis pain syndrome) reduction; 326 Hz, the frequency of miracle, the DNA recovery, and the frequency required for the resonance between the human body and the nature; 528 Hz, the frequency required for the frequency which most accords with A′ nature of Verdi; 432 Hz, the frequency required to turn sorrow into joy and to freed from guilt and fear; 369 Hz, the frequency necessary for purifying trauma experience and promoting change; 417 Hz, the frequency necessary for the communications, understanding, patience and love promotion, and the strengthening relation; 639 Hz, the frequency required for delivery by the power of self-expression leading to a pure and stable life; 741 Hz, the frequency required to wake up intuition; 852 Hz, or the like. Any one of these frequencies is input to the input module by a user, and electromagnetic energy satisfying the input data value is applied to the first transmitting coil 564a of the first electromagnetic energy generator 564 and the second transmitting coil 565a of the second electromagnetic energy generator 565, so that an electric field and magnetic field energy (RF energy) in the form of pulses are irradiated, and pulse-shaped pulsating quantum energy generated in a state in which the electric field and the magnetic field energy (RF energy) in opposite directions overlap and offset (dissipate) may be irradiated and transferred to a gaseous material including air passing through a flow path or a liquid material including water passing through a flow path.

FIGS. 13a, 13b, 13c, 13d, 13e, 13f, 13g, 13h, 13i, 13j, 13K, 13l, 13m, 13n are schematic drawings illustrating the following:
the shape of the quantum energy generation coil adopted to the third material supply unit 130 shown in FIG. 3, the shape of the quantum energy generation coil adopted to the third ion generator 270 shown in FIG. 5C,
the shape of the quantum energy generation coil adopted to first and second dissolving devices 310 and 320 shown in FIG. 6, the shape of the quantum energy generation coil adopted to the first and the second ion separator 400A and 400B shown in FIG. 7,
the shape of the quantum energy generation coil adopted to the third ion separator 400C shown in FIG. 8,
the shape of the quantum energy generation coil adopted to the first quantum energy transfer device 500A shown in FIG. 9,
the shape of the quantum energy generation coil adopted to the second quantum energy transfer device 500B shown in FIG. 10,
the shape of a quantum energy generation coil adopted to the third quantum energy transfer device 500C shown in FIG. 11,
and the shape of a quantum energy generation coil adopted to the fourth quantum energy transfer device 500D shown in FIG. 12.

FIG. 13a is a schematic drawing illustrating a shape of a quantum energy generating coil (first and second quantum energy generating coils) implemented in a shape of Cuffs coil. Referring to the attached drawings, the first quantum energy generating coil is wound a predetermined number of times clockwise or counterclockwise on a circumferential surface of one side of a lower portion in a circumferential direction of an outer surface of a vertically erected cylinder having a predetermined diameter, the second quantum energy generating coil is wound a predetermined number of times counterclockwise or clockwise on a circumferential surface of one side of an upper portion while being spaced apart from the first quantum energy generating coil by a predetermined distance in an upper direction, and winding directions of the first and second quantum energy generating coils are opposite to each other.

FIG. 13b is a schematic drawing illustrating a shape of the energy generating coils (the first and second energy generating coils) formed in Helmhelz coil shape. Referring to the accompanying drawings, the first energy generating coil is wound around one side of the left side in a clockwise direction or a counterclockwise direction in a circumferential direction of a surface of a horizontal cylinder having a predetermined diameter, the second energy generating coil is wound around one side of the right side in a counterclockwise direction or a clockwise direction while being spaced apart from the first energy generating coil by a predetermined distance in a right direction, and winding directions of the first and second energy generating coils are opposite to each other.

FIG. 13c is a schematic drawing illustrating a shape of a quantum energy generating coil (first and second quantum energy generating coils) implemented in Gradient saddle coil shape. Referring to the attached drawings, the first coil shape is a shape in which a quantum energy generating coil wire printed from a left end to a right end of an upper one side of an area of the entire circumferential surface of a section at a predetermined distance from an outer surface of horizontal or vertical cylinder having a predetermined diameter, that is, an outer surface area of a semicircular cylinder, extends from the left end to the right end in a circumferential direction, extends from the upper one side of the right end to the lower end, extends the coil wire from the right end to the left end, extends from the lower end to the coil wire of the upper end in a downward direction at a predetermined interval (first winding), extends the coil from the left end to the right end in a downward direction.

The shape of the second quantum energy generating coil is such that the winding direction of the first quantum energy generating coil is opposite to the winding direction of the first quantum energy generating coil. The second quantum energy generating coil is wound in a manner of extending the quantum energy generating coil printed from the right end of one side of the upper portion of the outer surface of the horizontal or vertical cylinder having a predetermined diameter to the left end in the circumferential direction, extending the coil from the left end to the lower end, extending the coil from the left end to the right end, extending the coil from the right lower end to the coil from the upper end in the downward direction at a predetermined interval (first winding), extending the coil from the right end to the left end at a predetermined interval in the downward direction at a predetermined interval from the first winding, winding the coil from the upper portion to the lower portion at a predetermined interval, and extending the coil from the left one side to the right one side.

FIGS. 13c and 13d are schematic drawings illustrating the shapes of the quantum energy generating coils (first and second quantum energy generating coils) implemented in the shape of Gradient saddle coil and Uniform saddle coil. Referring to the attached drawings, the shape of the first coil is similar to the shape of Gradient saddle coil illustrated in FIG. 4b, wherein the shape of the first coil is a shape in which a quantum energy generating coil wire printed from the left end to the right end of one side of an upper portion of an outer surface area of a horizontal or vertical cylinder having a predetermined diameter to a half area of the entire circumferential surface area of a section at a predetermined distance, that is, the outer surface area of the semicircular cylinder extends from the upper portion of the left end to the upper portion of the right end in the circumferential direction, the coil wire extends from the upper portion of the right end to the lower end of the right end, the coil wire extends from the lower end of the right end to the lower end of the left end, and the coil wire extends from the lower end of the left end to the upper end of the left at regular intervals, it is a shape that winds clockwise to the center.

The shape of the second quantum energy generating coil is such that the winding direction of the first quantum energy generating coil is opposite to the winding direction of the first quantum energy generating coil. The shape of the first coil is a shape in which a quantum energy generating coil wire printed from the right end to the left end of one side of an upper portion of an outer surface area of a horizontal or vertical cylinder having a predetermined diameter to a half area of the entire circumferential surface area of a section at a predetermined distance, that is, the outer surface area of the semicircular cylinder extends from the upper portion of the right end to the upper portion of the left end in the circumferential direction, the coil wire extends from the upper portion of the left end to the lower end of the left end, the coil wire extends from the lower end of the left end to the lower end of the right end, and the coil wire extends from the lower end of the right end to the upper end of the right at regular intervals, it is a shape that winds counterclockwise to the center.

FIG. 13e is a schematic drawing illustrating a shape of quantum energy generation coils (first and second quantum energy generation coils) implemented in a shape of Toroid coil.

Referring to the attached drawings, the first quantum energy generation coil is wound in a clockwise direction or a counterclockwise direction in Helical coil manner in a circumferential direction of a ring-shaped outer surface having a predetermined diameter.

The second quantum energy generation coil is wound in a clockwise direction or a counterclockwise direction in Helical coil manner in a circumferential direction of a ring-shaped outer surface having a predetermined diameter, and is the shape wound in the opposite direction of the winding direction of the first quantum energy generation coil.

FIG. 13f is a schematic drawing illustrating a shape of a quantum energy generating coil (first and second quantum energy generating coils) implemented in Trigger coil shape. Referring to the attached drawings, the shape of the first quantum energy generating coil is a shape in which a predetermined number of turns are wound clockwise or counterclockwise in a central direction from an outer edge portion on a circular plate as illustrated in FIG. 4E, and the second quantum energy generating coil is a shape in which a predetermined number of turns are wound counterclockwise or clockwise inside or outside a concentric circle of the first quantum energy generating coil in a direction opposite to a winding direction of the first quantum energy generating coil.

FIG. 13g is a schematic drawing illustrating a shape of Zigzag-shaped quantum energy generating coil (first and second quantum energy generating coils). Referring to the attached drawings, the shape of the first quantum energy generating coil is wound a predetermined number of turns in the zigzag direction in the vertical direction or the horizontal direction as shown in FIG. 4f, and the shape of the second quantum energy generating coil is wound a predetermined number of turns in the zigzag direction in the vertical direction or the horizontal direction in the opposite direction of the winding direction of the first quantum energy generating coil.

FIG. 13h is a schematic drawing illustrating a shape of a quantum energy generating coil (first and second quantum energy generating coils) implemented in Temple-shaped coil. Referring to the attached drawings, Temple-shaped coil is a shape similar to Zigzag shape, and the first temple-shaped coil is a shape wound in a circular shape to have a predetermined diameter with Zigzag shape coil, and the second temple-shaped coil is a shape wound in a circular shape to have a predetermined diameter with Zigzag shape coil inside or outside a concentric circle of the first temple-shaped coil, and a flow direction of a current is a shape wound to be opposite to each other.

FIG. 13i is a schematic drawing illustrating a shape of a quantum energy generation coil (first and second quantum energy generation coils) implemented in a motor stator-type coil shape. Referring to the attached drawings, the shape of the first quantum energy generation coil is a shape in which a small circular shape comes into surface contact with the first quantum energy generation coil to form a plurality of small circular shapes having a predetermined diameter and a circular shape having a predetermined diameter, and the shape of the second quantum energy generation coil is a shape in which one side of the plurality of coils comes into surface contact with the second quantum energy generation coil in a concentric circle formed by surface contact with the plurality of first quantum energy generation coils or a circular shape having a predetermined diameter formed by surface contact with the second quantum energy generation coil.

Flow directions of the current of the first and the second quantum energy generation coils are the shapes wound to be opposite to each other.

FIG. 13j is a schematic drawing illustrating a shape of a quantum energy generating coil (first and second quantum energy generating coils) implemented in a straight-angle shape coil or an edge-wise voice shape coil. Referring to the attached drawings, the straight-angle shape coil or the edge-wise voice shape coil has a shape similar to the shape of Cuffs coil illustrated in FIG. 13a, and the first quantum energy generating coil has a shape in which a predetermined number of turns are wound clockwise or counterclockwise in a circumferential direction of a horizontal cylindrical outer surface having a predetermined diameter, and the second quantum energy has a shape in which a winding direction is opposite to that of the first quantum energy generating coil and a predetermined number of turns are wound counterclockwise or clockwise in the circumferential direction of the cylindrical outer surface.

FIG. 13k is a schematic drawing illustrating a shape of a quantum energy generating coil (first and second quantum energy generating coils) implemented in Toroidal coil shape. Referring to the attached drawings, the first quantum energy generating coil shape has a concentric circular shape of a predetermined distance, and is formed such that a predetermined number of coils are wound in a clockwise direction or a counterclockwise direction in a circumferential direction of an outer surface of a ring at a predetermined diameter, spaced apart from each other by a predetermined distance. The second quantum energy generating coil shape has a concentric circular shape of a predetermined distance, and is formed such that a predetermined number of coils are wound in a clockwise direction or a counterclockwise direction in a circumferential direction of an outer surface of a ring at a predetermined diameter, spaced apart from each other by a predetermined distance, flow directions of the current of the first and the second quantum energy generation coils are the shapes wound to be opposite to each other.

FIG. 13l is a schematic drawing illustrating a shape of a quantum energy generation coil (first and second quantum energy generation coils) implemented in Solenoid coil shape. Referring to the attached drawings, Solenoid coil shape is a shape similar to a spring shape or Helical shape, and the first quantum energy generation coil is wound a predetermined number of times in a clockwise direction or a counterclockwise direction in a circumferential direction of a horizontal cylindrical center line outer surface having a predetermined diameter, and the second quantum energy generation coil is wound a predetermined number of times in a counterclockwise direction or a clockwise direction on the circumferential surface in a manner of winding the first quantum energy generation coil and the winding direction to be opposite to each other, and current flow directions in the first and second quantum energy generation coils are opposite to each other.

FIG. 13m is a schematic drawing illustrating shapes of quantum energy generating coils (first and second quantum energy generating coils) implemented in a modified RF coil shape. Referring to the attached drawings, the first quantum energy generating coil shape is a shape in which a plurality of coils are wound around an outer surface of a rectangular or elliptical shape having a predetermined area in a clockwise direction or a counterclockwise direction at intervals on a concentric circle, and winding directions of adjacent coil groups are opposite to each other, the second quantum energy generating coil shape is a shape in which a plurality of coils are wound around an outer surface of a rectangular or elliptical shape having a predetermined area in a counterclockwise direction or a clockwise direction at intervals on a concentric circle, and winding directions of the first and second quantum energy generating coils are opposite to each other.

FIG. 13n is a schematic drawing illustrating a shape of a quantum energy generating coil (first and second quantum energy generating coils) implemented in Helical or Resonant coil shape. Referring to the attached drawings, Helical or Resonant coil shape has a shape (circular plate material) having a predetermined width and length, and is manufactured in the following order: a hole having a predetermined diameter is formed in the center of one end portion of a circular plate (ring shape) separated by cutting one side, a hole having a predetermined diameter is formed in the perforated hole, and a fixture having a hole having a predetermined diameter is formed in the center of a cross section of both end portions of the cylindrical shape, so that an opening portion of the first circular plate is directed to the right side, one end portion of the fixture is inserted into the perforated hole and riveted, or a bolt and a nut are fastened and fixed, and a second circular plate is formed on the other end portion of the fixture in a direction opposite to that of the first circular plate to insert and fix the perforated hole of the second circular plate into the fixing.

Although the preferred embodiments of the present invention have been described in detail above, the present invention is not limited to the above-described specific preferred embodiments, and any person skilled in the art to which the present invention pertains may perform various modifications without departing from the gist of the present invention claimed in the following claims, and such modifications are within the scope of the claims.

1-20. (canceled)

21-31. (canceled)

32. An apparatus, comprising:
a first dissolving device 310 including
a first chamber 315,
a disperser 315a installed inside the first chamber 315,
a first defoamer 318, wherein the first defoamer 318 includes a first quantum energy generator consisting of a first quantum energy generating coil 316a, a second quantum energy generating coil 316b and a pulsed power supply unit 317,
a first circulating pump 311 installed in connection with the first chamber 315 inlet,
a venturi ejector 313 installed between the first chamber 315 inlet and the first circulating pump 311,
a first flow control valve 312 installed between the venturi ejector 313 and the first circulating pump 311, and
a first circulation tube 319 installed in connection with the first chamber 315 outlet; and
a second dissolving device 320 including
a second chamber 324,
a second defoamer 327, wherein the second defoamer 327 includes a second quantum energy generator consisting of a first quantum energy generating coil 325a, a second quantum energy generating coil 325b and a pulsed power supply unit 326a,
a third quantum energy generating coil 325c installed inside the second chamber 324, and installed downward at intervals from the first and second quantum energy generating coils 325a and 325b,
a Trigger voltage generator 326b supplied power to the third quantum energy generating coil 325c, and connected to the pulsed power supply unit 326a with a conducting wire,
a second circulating pump 321 installed between the second chamber 324 inlet and the first circulation tube 319,
a second flow control valve 322 installed between second chamber 324 inlet and the second circulating pump 321, and
a second circulation tube installed in connection with the second chamber 324 outlet.

33. The apparatus according to claim 32, wherein
the disperser 315a installed in the inner center of the first chamber 315 includes cylindrical pores of less than 1 mm with a certain diameter and length,
by operating the first circulation pump 311, the solution is sucked and pressurized, and hydrogen ions (H+, H−), oxygen ions (O+, O−) or nitrogen ions (N+, N−, NO) generated in the ion generator are supplied in the process of passing through the venturi ejector 313, and
air bubbles mixed in the solution are primarily destroyed and decompressed during the process of passing through the disperser 315a having the cylindrical pores of less than 1 mm.

34. The apparatus according to claim 33, wherein
the first and second quantum energy generating coils 316a and 316b are attached to the outer surface of the first chamber 315,
the first and second quantum energy generating coils 316a, 316b are installed to face each other at interval, and the wound coil directions are opposite to each other, and
the first and second quantum energy generating coils 316a and 316b include Uniform saddle coils.

35. The apparatus according to claim 34, wherein
a high voltage in the form of a pulse generated by the pulsed power supply unit 317 of the first quantum energy generator supplies to the first and second quantum energy generating coils 316a and 316b,
between the coils, the electromagnetic field of the pulse form of the opposite direction is generated, and the electromagnetic field of the pulse form of the opposite direction at the center distance are overlapped and offset (dissipated), and a quantum energy is generated in the state of the zero magnetic field, and it is irradiated inside the first chamber 315, and
the quantum energy irradiates to the solution inside the first chamber 315 to depocalyse (destroy) the air bubbles contained in the solution, as the result, the hydrogen ions (H+, H−), oxygen ions (O+, O−) or nitrogen ions (N+, N−, NO) are primary dissolved.

36. The apparatus according to claim 35, wherein
power of the second circulation pump 321 is on, the second circulation pump 321 is operated so that the process fluid in which hydrogen ions (H+, H−), oxygen ions (O+, O−) or nitrogen ions (N+, N−, NO) are primarily dissolved in the first dissolving device 310 is sucked and pressurized by the second circulation pump 321 and introduced into the second chamber 324 to which the second quantum energy is irradiated through the second flow rate adjusting valve 322.

37. The apparatus according to claim 32, wherein
the second chamber 324 has a cylindrical shape having a predetermined diameter and a predetermined length so that a first quantum energy generating coil 325a having Solenoid coil shape is installed to be in surface contact with the inner surface, and is insulated from the inner surface of the cylindrical second chamber 324, and
the second quantum energy generating coil 325b having Trigger coil shape is installed at intervals from the first quantum energy generating coils 325a.

38. The apparatus according to claim 32, wherein
the third quantum energy generating coil 325c has a modified RF coil shape.

39. The apparatus according to claim 32, wherein
the first and second quantum energy generating coils 325a and 325b are connected to a pulsed power supply unit 326a through a conducting wire,
the third quantum energy generating coil 325c is connected to the Trigger voltage generator 326b through the conducting wire, and
the pulsed power supply unit 326a and the Trigger voltage generator 326b are also connected through a conducting wire.

40. The apparatus according to claim 39, wherein
the third quantum energy generating coil 325c starts discharging while pulse energy is transferred between the first and second quantum energy generating coils 325a and 325b, and
the third quantum energy generating coil 325c starts main discharge at a lower voltage than the high voltage pulse applied to the first quantum energy generating coil 325a and the second quantum energy generating coil 325b.

41. The apparatus according to claim 40, wherein
the pulsed power supply unit 326a generates pulse-type high voltages to the first quantum energy generating coil 325a and the second quantum energy generating coil 325b installed such that winding directions of the coils are opposite to each other through a conducting wire,
pulse-type magnetic fields in opposite directions are generated in the first quantum energy generating coil 325a and the second quantum energy generating coil 325b at an angle of 90° with respect to a flow direction of a current, the pulse-type magnetic fields in opposite directions are overlapped and offset (dissipated) at a center distance of the first quantum energy generating coil 325a and the second quantum energy generating coil 325b, and quantum energy is generated in a zero magnetic field state and irradiated to the introduced fluid, and
as a result, a shock wave is generated by the discharge, and at the same time, bubbles containing hydrogen ions (H+, H−), oxygen ions (O+, O−) or nitrogen ions (N+, N−, NO), which are not destroyed in the first dissolving device 310 passing through between the first quantum energy generating coil 325a, the second quantum energy generating coil 325b, and the third quantum energy generating coil 325c, are destroyed by the discharge shock wave, and the water hammer pressure generated by the destruction of the bubbles efficiently further destroys the bubbles adjacent to the discharge shock wave.
